(19) United States
(12) Patent Application Publication
(10) Pub. No.: US 2022/0280746 A1
(43) Pub. Date: Sep. 8, 2022

(54) Catheter

(71) Applicant: Wiesconcepts, LLC, York, PA (US)

(72) Inventor: Jon Wiesman, Middle River, MD (US)

(73) Assignee: Wiesconcepts, LLC, York, PA (US)

(21) Appl. No.: 17/752,507

(22) Filed: May 24, 2022

Related U.S. Application Data
(63) Continuation-in-part of application No. 17/214,388, filed on Mar. 26, 2021.
(60) Provisional application No. 63/000,919, filed on Mar. 27, 2020.
(60) Provisional application No. 63/135,164, filed on Jan. 8, 2021.

(51) Int. Cl. A61M 19/00; A61M 25/00

(52) U.S. Cl. CPC A61M 19/00; A61M 25/0084; A61M 2210/1089; A61M 2210/1085; A61M 2025/0089

(57) ABSTRACT

A catheter assembly having the major components: a cap, an introducer tip, four different variations of grip housing, a tube gripper and a catheter. The variations of the housing connected to the catheter assembly allows for safe and effective delivery of the anesthetics to various bodily orifices. The catheter is slowly advanced further while irrigating and dispensing the medication and/or lidocaine gel from the syringe. Once the desired depth is reached the practitioner can mark off the depth in centimeters on the catheter using a variation of the marker.

### CROSS REFERENCE TO RELATED APPLICATION

This application is a continuation-in-part of the U.S. application Ser. No. 17/214,388 filed on Mar. 26, 2021, which claims the benefit of the filling date under 35 U.S.C. 119 of the U.S. Provisional Patent Application No. 63/000,919, filed on Mar. 27, 2020, and to U.S. Provisional Patent Application No. 63/135,164, filed on Jan. 8, 2021.

### FIELD OF THE INVENTION

The field of the invention is a catheter assembly and, more particularly, a graduated tubular style catheter/protective sheath assembly with different variations.

### BACKGROUND

Various medical procedures are performed requiring access to internal cavities and organs in a living being. A procedure such as biopsy, endoscopy, or culture sampling, may require, for example, directing a medical device such as an endoscope, biopsy device, or culture swab into a tract, for example, the urinary tract to access the bladder region, or into the nasal cavity to access the nasopharynx or nasopharyngeal region. Such methods are uncomfortable and painful for the patient.

The only known commercially available method of anesthetizing the urethra is using an oral tip and/or a modified catheter tip syringe. The above method only provides minimal coverage for anesthetizing a longer or larger tract, leaving the majority of the region to be evaluated untouched/unaffected by the anesthesia.

As of 2019, there are more than 13,044 actively practicing urologists in the United States. Patient studies have determined that the majority of pain experienced with a urological examination occurs when approaching to and or passing an object through the urethral sphincter. On behalf of anesthetizing the urethra, the syringe tip enters into the urethra, passing forward the bulbous penis to deliver the anesthetizing jelly, although due to vascular pressure, the jelly is pushed back, ultimately anesthetizing only about the first 20% of the urethra. According to the instructions for use of such a commercially available product, once the anesthetic is dispensed, a penile clamp is applied for about 30 minutes ahead of the examination.

For properly anesthetizing nasopharyngeal airway, the only known method is using a liquid spray/mist format, whereby it is inhaled through the nostril.

In the case of the nasopharyngeal airway, a spray format is used, which is inhaled through the nostril, coverage is again minimal at best; if the patient has a sinus blockage of the nasopharyngeal airway (minimal inhale) there will be regions totally untreated due to the reduced drawing pressure.

Regardless of the situation, the above methods of applying anesthesia opens the patient to significant discomfort regardless of the organ, thus reduces the ease of the initial evaluation for the practitioner. We believe this added stress can easily bring forward a missed diagnosis and what makes this closed system device so unique is the overall assembly, which enables the urethra and/or nasopharyngeal airway to be completely anesthetized. Further, less patient stress can improve a practitioner's confidence.

What is needed is a catheter assembly which includes a protective sheath assembly with different variations that can secure the catheter in places while facilitating delivery of anesthetic to the entire tract or body region, and further provide a route for fluid and device access. Which is further designed to reduce the onset of pain associated with a urological examination and allows for complete anesthetization to be achieved in less than 5 minutes. Moreover, to overall improve how an office urological exam is prepped.

### SUMMARY

A novel tubular type/protective sheath assembly is provided and is used for intradermal delivery of an active agent, for the primary purpose of anesthetizing a region of the body ahead of an evaluation and/or elected procedure and/or a medication for the treatment of a said affliction.

The primary use of the graduated tubular style catheter/protective sheath assembly will be to anesthetize the entire urethra up to the neck of the bladder and urethral sphincter, and further into the bladder and/or reproductive tract, although it can also be easily modified to accommodate use in the nasopharyngeal airway canal. In some embodiments, the protective sheath assembly may be provided to accommodate an atomizing spray nozzle, for treating an air space within the body, such as the nasal cavity.

The secondary use of this device is to act as a protective sheath reducing the risk of infection and/or damage to the epithelial lining of a region. The protective sheath assembly will facilitate the passage of a variety of medical devices therethrough, such as an endoscope, biopsy needle and/or biopsy punch, surgical fiber and/or aspiration needle, culture swab and/or brush.

Such a medical device directed through the protective sheath may, in an embodiment, be connected to a UV light source, and is an optical fiber that can transmit the UV light through the length of the surgical fiber, delivering it at the distal tip of the surgical fiber to treat an area with

UV light, particularly UV-B and UV-C wavelengths, as they are known to have germicidal properties.

The disclosure of various embodiments described herein further provides an effective and less invasive approach for the direct delivery of therapeutic agent (i.e., a 2% lidocaine regardless of format; a formulated medication and/or chemotherapy); and also enables the practitioner, so that once the embodiment is seeded in place, the protective sheath assembly provides protection to permit other devices to easily access through a lumen. Thus the invention can lessen the number of times a device re-enters the lumen, and thereby reduces the likelihood of inducing irritation and/or the introduction of bacteria which may cause a possible urinary tract infection (UTI) or upper respiratory infection (URI) following procedures that require introduction of devices such as endoscopes, biopsy needles, surgical fibers such as those utilized for a laser procedure, and/or culture sampling devices such as swabs or brushes. Furthermore, the various embodiments described herein may enable the practitioner to incorporate a removable atomizing spray nozzle in the tip of the protective sheath assembly for anesthetization and procedures in the nasal pharyngeal region.

In an exemplary embodiment, there is provided a protective assembly having a protective sheath assembly with a tubular catheter body having a proximal and distal end, and at least one connection fitting at the proximal end; and a holding device assembly having a housing, an introducer tip, and a retention device. The protective assembly may further comprise a syringe that can be attached to the connection fitting. The syringe may contain a medicament.

In an embodiment, the connection fitting is a Y connector with a first port for connection to a syringe, and a second port that is a funnel type opening and may feature a removable or releasable plug to seal the opening. In an exemplary embodiment, the funnel opening can accommodate up to a 14 french device therethrough. The first port may optionally be provided with a one-way valve.

In an exemplary embodiment, the housing of the holding device has a catheter receiving passageway therethrough with an internal volume containing an amount of lubricating jelly, and may optionally have a removable cap.

In an exemplary embodiment, the retention device can seal a first end of the housing, and the introducer tip is configured to seal a second end of the housing, such that the retention device and introducer tip cooperatively seal lubricating jelly within the volume of the housing. The tubular catheter body can pass through and be advanced through each of the retention device, the housing and the introducer tip, as each of the retention device and introducer tip have a slit seal to accommodate passage of the tubular catheter body. The slit seal of the retention device can also serve to grip the tubular catheter body as it passes through and provides frictional resistance to the free movement of the catheter body relative to the retention device.

In an exemplary embodiment, the housing can have laterally extended gripping surfaces. In an embodiment, the housing can have a rectangular prism shape, with upper and lower gripping surfaces. The upper and lower gripping surfaces may be planar, and may be recessed slightly into the housing, to provide a lip about the perimeter of each of the upper and lower gripping surfaces.

In an exemplary embodiment, the tubular catheter body has graduation marks to indicate the penetration depth of the tubular catheter body and may have a marker in sliding relationship with the tubular catheter body.

In an exemplary embodiment, the tubular catheter body is configured to pass and be advanced through the housing and the retention device that is a flexible ring that compresses a portion of the housing against the tubular catheter body and may rest against the tubular catheter body.

In an exemplary embodiment, the housing has a pair of projecting gripping surfaces that can be urged against the tubular catheter body by the retention device when in a first position, and when the retention device is in a second position, the pair of projecting gripping surfaces are not urged against the tubular catheter body.

In an exemplary embodiment, the housing has a pair of tapered sides, and the retention device, when in a first position, is on the tapered sides, and serves to compress the tapered sides against the tubular catheter body, and frictionally restrains the catheter from being freely moved relative to the housing. Further, when the retention device is in a second position, it does not compress the tapered sides against the tubular catheter body and allows the tubular catheter body to be freely moved relative to the housing.

In an exemplary embodiment, there is provided a protective assembly for anesthetizing the length of a urethra of a living being, having a protective sheath assembly with a holding device assembly having a housing, an introducer tip, and a retention device; and a tubular catheter body with a proximal end having at least one connection fitting, and a distal end having at least one opening, and an interior lumen extending through tubular catheter body; and an anesthetizing medicament device means connected to the at least one connection fitting and provided with an anesthetizing medicament, the anesthetizing medicament being advanced by the anesthetizing medicament device means through the interior lumen and exiting the at least one opening of the tubular catheter body to treat a targeted area of the body.

In another exemplary embodiment, there is provided a protective assembly for anesthetizing the nasal cavity of a living being, having a protective sheath assembly with: a holding device assembly having a housing, an introducer tip, and a retention device; and a tubular catheter body with a proximal end having at least one connection fitting, and a distal end having at least one opening configured to release a mist, and an interior lumen extending through tubular catheter body; and an anesthetizing medicament device means connected to the at least one connection fitting and filled with an anesthetizing medicament, the anesthetizing medicament being advanced by the anesthetizing medicament device means through the interior lumen and exiting the at least one opening as a mist to locally anesthetize a target area of the body.

In another exemplary embodiment there is provided a protective assembly for treating an infection in a living being having a protective sheath assembly with: a holding device assembly having a housing, an introducer tip, and a retention device; and a tubular catheter body with a proximal end having at least one connection fitting, and a distal end having at least one opening, and an interior lumen extending through tubular catheter body; and a disinfecting means configured to deliver a UV treatment, the disinfecting means being advanced through the interior lumen and exiting the at least one opening and to deliver the UV treatment locally to a targeted area. The disinfecting means can be a surgical fiber having a proximal fiber end that is in contact with a UV light source, such that the distal fiber end serves to radiate UV light to treat an area. The UV light source may produce light having a wavelength range selected from at least UV-B, UV-C, and combinations thereof.

In another exemplary embodiment, the catheter assembly provides different variations of a grip housing design in order to control the movement of the catheter. The catheter assembly is similar to what is described above. However, the catheter assembly includes four different variations of grip housing which provides unique characteristic for the device when in use, to irrigate the urethra with an anesthetic from the glans to the bladder sphincter.

### BRIEF DESCRIPTION OF DRAWINGS

The invention will now be described by way of example with reference to the accompanying Figures of which:

FIG. 1 is a perspective view of an exemplary embodiment of the protective sheath assembly having a luer connection at the proximal end, and an embodiment of the grip housing having lateral wing gripping surfaces;

FIGS. 2 depicts a plan view of the embodiment of FIG. 1, showing the catheter separate from the holding device;

FIG. 3 depicts the device of FIG. 3, with the catheter directed through the holding device;

FIG. 4 depicts an exploded view of the components of an embodiment of the holding device;

FIG. 5 is an enlarged isolated view of the introducer tip, and showing a slit seal at the distal end of the introducer tip;

FIG. 6 is an end view of the isolated distal tip;

FIG. 7 is an enlarged view of the holding device of FIG. 4;

FIG. 8 is an isolated view of an embodiment of the tubular catheter body;

FIG. 9 is a perspective view of another exemplary embodiment of the protective sheath assembly having a Y connector at the proximal end, and an embodiment of the grip housing having lateral wing gripping surfaces;

FIG. 10 is an enlarged view of the isolated Y connector;

FIGS. 11 and 12 depict another exemplary embodiment of the protective sheath assembly having a Y connector at the proximal end, and an embodiment of the grip housing having a repositionable retention device;

FIGS. 13-15 depict isolated views of the Y connector having a luer connection and a funnel type opening with a releasable plug;

FIG. 16 depicts another exemplary embodiment of the protective sheath assembly having a luer connector at the proximal end, and an embodiment of the grip housing having a repositionable retention device;

FIGS. 17 and 18 depict isolated views of the holding device of the embodiment of FIG. 16, showing the repositioning of the retention device;

FIG. 19 depicts another view of the device of FIG. 16, with the syringe removed;

FIG. 20 is an enlarged perspective view of the introducer tip of the embodiment of FIG. 16;

FIG. 21 is an enlarged perspective view of the removable cap of the embodiment of FIG. 16;

FIG. 22 is an enlarged view of the holding device of the embodiment of FIG. 16;

FIG. 23 is an enlarged view of the marker and graduated markings of the embodiment of FIG. 16;

FIG. 24 is a cross-sectional view of the holding device and distal portion of the catheter of the embodiment of FIG. 16, taken along line 24-24 of FIG. 17;

FIG. 25 is a cross-section view of the holding device taken along line 25-25 of FIG. 24;

FIG. 26 is a cross-sectional view of the holding device and distal portion of the catheter of the embodiment of FIG. 16, taken along line 26-26 of FIG. 18, and showing the positioning of the introducer tip within a tract of a living being;

FIG. 27 is an enlarged perspective view of the holding device of the embodiment of FIG. 16, depicting the catheter being extended out the end of the introducer tip;

FIGS. 28 -29 are perspective views of the isolated grip housing of the embodiment of FIG. 16;

FIG. 30 is a cross-sectional view of the isolated holding device taken along line 30-30 of FIG. 28;

FIG. 31 is another cross-sectional view of the isolated holding device taken along line 31-31 of FIG. 28;

FIGS. 32 depicts an embodiment of a graduated tubular style catheter/protective sheath assembly having a luer connection at the proximal end, and an open distal tip;

FIG. 33 depicts an enlarged section view of the proximal end of the embodiment of FIG. 32;

FIG. 34 depicts an enlarged section view of the open distal end of the embodiment of FIG. 32;

FIG. 35 depicts an isolated section view of the distal end of the catheter of FIG. 32;

FIG. 36 depicts a cross-sectional view of the isolated distal end of the catheter of FIG. 32, taken along line 36-36 of FIG. 35;

FIG. 37-39 depict isolated section views of the proximal end of the graduated tubular style catheter/protective sheath assembly of FIG. 32 and having a luer slip connector;

FIG. 40 depicts still another exemplary embodiment of the protective sheath assembly having a luer connector at the proximal end, and an embodiment of the grip housing having a repositionable retention device and a pair of gripping surfaces that are urged against the catheter by the retention device when in a first position;

FIGS. 41 depict an isolated view of the holding device of the embodiment of FIG. 40, showing the repositionable retention device in a position to cause the gripping of the catheter;

FIG. 42 depicts the holding device of FIG. 41, with the cap removed to expose the introducer tip;

FIG. 43 depicts the holding device of FIG. 41, with the repositionable retention device in a position to release the grip on the catheter, and thereby allow the free movement of the catheter within the holding device;

FIG. 44 depicts an isolated view of the grip housing of the embodiment of FIG. 40, showing the gripping surfaces and lateral wings;

FIG. 45 depicts a cross-sectional view of the isolated grip housing of FIG. 40, taken along line 45-45 of FIG. 44;

FIGS. 46 depicts an embodiment of a graduated tubular style catheter/protective sheath assembly having a luer connection at the proximal end, and a closed distal tip;

FIG. 47 depicts an enlarged section view of the proximal end of the embodiment of FIG. 46;

FIG. 48 depicts an enlarged section view of the closed distal end of the embodiment of FIG. 46, showing opening in the sidewall;

FIG. 49 depicts an isolated section view of the proximal end of the graduated tubular style catheter/protective sheath assembly of FIG. 46;

FIG. 50 depicts an isolated section view of the distal end of the graduated tubular style catheter/protective sheath assembly of FIG. 46;

FIG. 51 depicts still another exemplary embodiment of the protective sheath assembly having a luer connector at the proximal end, and an embodiment of the grip housing having a rectangular prism shaped body providing upper and lower gripping surfaces that are recessed into the body to aid in gripping;

FIGS. 52 depict isolated views of the holding device of the embodiment of FIG. 51, showing the recessed gripping surface;

FIG. 53 depicts the holding device of FIG. 52, with the cap removed to expose the introducer tip;

FIG. 54 is an end view of the holding device of FIG. 51, depicting the slit valve of the introducer tip;

FIG. 55 is an enlarged perspective view of the holding device of the embodiment of FIG. 51, depicting the closed end catheter being extended out the end of the introducer tip;

FIG. 56 is a cross-sectional view of the holding device and distal portion of the catheter of the embodiment of FIG. 51, taken along line 56-56 of FIG. 55, and showing the positioning of the introducer tip within a tract of a living being, and partial extension of the catheter into the tract;

FIG. 57-59 depict an embodiment of the retention device for the embodiments of FIGS. 7 and 51, with the cross-section view of FIG. 58 taken along the line 58-58 of FIG. 57; and

FIG. 60 depicts representative devices that can be used with the various embodiments of the invention described herein, depicting a light source and surgical fiber, or a biopsy needle.

FIG. 61 is a perspective view of an exemplary embodiment of the catheter;

FIG. 62 depicts an exploded view of the embodiment of FIG. 61;

FIG. 63 depicts the device of FIG. 62, with the catheter directed through the holding device;

FIG. 64 depicts an exploded view of the holding device of FIG. 63;

FIG. 65 depicts a top view of the holding device of FIG. 64;

FIG. 66 is an isolated view of an embodiment of the tubular catheter body of FIG. 63;

FIG. 67 depicts an embodiment of a graduated tubular style catheter/protective sheath assembly of FIG. 66;

FIG. 68 depicts an enlarged view of the proximal end of FIG. 67;

FIG. 69 depicts an enlarged section view of the open distal end of FIG. 67;

FIG. 70 depicts an enlarged section view of the open distal end of FIG. 67;

FIG. 71 depicts an enlarged isolated view of the introducer tip of FIG. 63;

FIG. 72 depicts a front view of the introducer tip of FIG. 71;

FIG. 73 depicts a rear view of the introducer tip of FIG. 72;

FIG. 74 depicts an enlarged rear view of the introducer tip of FIG. 73;

FIG. 75 depicts a cross-sectional view of the introducer tip of FIG. 74;

FIG. 76 depicts a top front perspective view of the holding device of FIG. 65;

FIG. 77 depicts a top perspective view of the holding device in FIG. 76;

FIG. 78 depicts a top vertical view of the holding device in FIG. 77;

FIG. 79 depicts a bottom view of the holding device in FIG. 76;

FIG. 80 depicts a bottom cross-sectional view of FIG. 79;

FIG. 81 depicts a top cross-sectional view of FIG. 80;

FIG. 82 depicts a front cross-sectional view of FIG. 81;

FIG. 83 depicts a front view of the holding device of FIG. 82;

FIG. 84 depicts a front elevated view of the holding device of FIG. 83;

FIG. 85 depicts a side view of the tube gripper;

FIG. 86 depicts a front view of the tube gripper in FIG. 85;

FIG. 87 depicts a vertical cross-sectional view of the tube gripper of FIG. 86;

FIG. 88 depicts a horizontal cross-sectional view of the tube gripper of FIG. 87;

FIG. 89 depicts an enlarged perspective view of the removable cap of FIG. 62;

FIG. 90 depicts a rear perspective view of the removable cap of FIG. 89;

FIG. 91-93 depict an embodiment of the catheter receiver, with the cross-section view of FIG. 92 taken along the line 92-92 of FIG. 91;

FIG. 94 depicts the catheter assembly being directed into the urethral opening, and advancing the catheter towards the bladder;

FIG. 95 depicts an isolated view of the holding device of the embodiment of FIG. 65;

FIG. 96 depicts the holding device of FIG. 95, with the cap removed to expose the introducer tip;

FIG. 97 depicts an enlarged perspective view of the holding device of the embodiment 61, depicting the closed end catheter being extended out the end of the introducer tip of FIG. 96;

FIG. 98 depicts a side cross-sectional view of FIG. 97;

FIG. 99 depicts top cross-sectional view of FIG. 98

FIG. 100 depicts a top cross-sectional view of the tube gripper in first position;

FIG. 101 depicts a front cross-sectional view of FIG. 100;

FIG. 102 depicts a top cross-sectional view of the tube gripper in second position; and

FIG. 103 depicts a front cross-sectional view of FIG. 102.

FIG. 104 depicts a front top left perspective view of a variation of the housing of FIG. 103;

FIG. 105 depicts a rear top left perspective view of one variation the housing of FIG. 104;

FIG. 106 depicts a top perspective view of one variation of the housing of FIG. 105;

FIG. 107 depicts a left side perspective view of one variation of the housing of FIG. 106;

FIG. 108 depicts a rear perspective view of one variation of the housing of FIG. 107;

FIG. 109 depicts a front perspective view of one variation of the housing of FIG. 108;

FIG. 110 is a front top left side perspective view of one variation of the housing of FIG. 109;

FIG. 111 is a bottom front perspective view of one variation of the housing of FIG. 110;

FIG. 112 is a left side cross sectional view of one variation of the housing of FIG. 111;

FIG. 113 is an enlarged left side cross sectional view of one variation of the housing of FIG. 112;

FIG. 114 is a front cross-sectional view of one variation of the housing of FIG. 113;

FIG. 115 is a front top left side perspective view of one variation of the housing of FIG. 114;

FIG. 116 is a rear top left side perspective view of one variation of the housing of FIG. 115;

FIG. 117 is a top perspective view of one variation of the housing of FIG. 116;

FIG. 118 is a left side perspective view of one variation of the housing of FIG. 117;

FIG. 119 is a rear perspective view of one variation of the housing of FIG. 118;

FIG. 120 is a front perspective view of one variation of the housing of FIG. 119;

FIG. 121 a front top left side perspective view of one variation of the housing of FIG. 120;

FIG. 122 is a bottom front perspective view of one variation of the housing of FIG. 121;

FIG. 123 is a left side cross sectional view of one variation of the housing of FIG. 122;

FIG. 124 is an enlarged left side cross sectional view of one variation of the housing of FIG. 123;

FIG. 125 is a front cross-sectional view of one variation of the housing of FIG. 124;

FIG. 126 is a top perspective view of a second variation of the housing of FIG. 125;

FIG. 127 is a left side perspective view of a second variation of the housing of FIG. 126;

FIG. 128 is rear perspective view of a second variation of the housing of FIG. 127;

FIG. 129 is a front top left side perspective view of a second variation of the housing of FIG. 128;

FIG. 130 is a rear top left side perspective view of a second variation of the housing of FIG. 129;

FIG. 131 is a front perspective view a second variation of the housing of FIG. 130;

FIG. 132 is a top rear left side perspective view of a second variation of the housing of FIG. 131;

FIG. 133 is a left side cross sectional view of a second variation of the housing of FIG. 132;

FIG. 134 is an enlarged left side cross sectional view of a second variation of the housing of FIG. 133;

FIG. 135 is a rear cross-sectional view of a second variation of the housing of FIG. 134;

FIG. 136 is a top front right side perspective view of a third variation of the housing of FIG. 135

FIG. 137 is a top rear right side perspective view of a third variation of the housing of FIG. 136;

FIG. 138 is a top view of a third variation of the housing of FIG. 137;

FIG. 139 is a left side perspective view of a third variation of the housing of FIG. 138;

FIG. 140 is a rear perspective view of a third variation of the housing of FIG. 139;

FIG. 141 is a rear view of a third variation of the housing of FIG. 140;

FIG. 142 is a top left rear view of a third variation of the housing of FIG. 141;

FIG. 143 is a top cross-sectional view of a third variation of the housing of FIG. 142;

FIG. 144 is a top rear cross-sectional view of a third variation of the housing of FIG. 143;

FIG. 145 rear cross-sectional view of the third variation of the housing of FIG. 144;

FIG. 146 is an alternative version of the catheter assembly of FIG. 145;

FIG. 147 is an alternative version of the marker of FIG. 146;

FIG. 148 is a top front right view of the marker of FIG. 147;

FIG. 149 is a front cross-sectional view of the marker of FIG. 148;

FIG. 150 is a left side cross-sectional view of the marker of FIG. 149;

FIG. 151 is a front bottom perspective view of the marker of FIG. 150;

FIG. 152 is a front perspective view of the marker of FIG. 151; and

FIG. 153 is a left side cross-sectional view of the marker of FIG. 152.

### DETAILED DESCRIPTION OF THE EMBODIMENT(S)

Exemplary embodiments of the invention will be described hereinafter in detail with reference to the attached drawings, wherein like reference numerals refer to like elements.

The invention may, however, be embodied in many different forms and should not be construed as being limited to the particular embodiments set forth herein; rather, these embodiments are provided so that the present disclosure will be thorough and complete, and will fully convey the concept of the disclosure to those skilled in the art.

### First Exemplary Embodiment

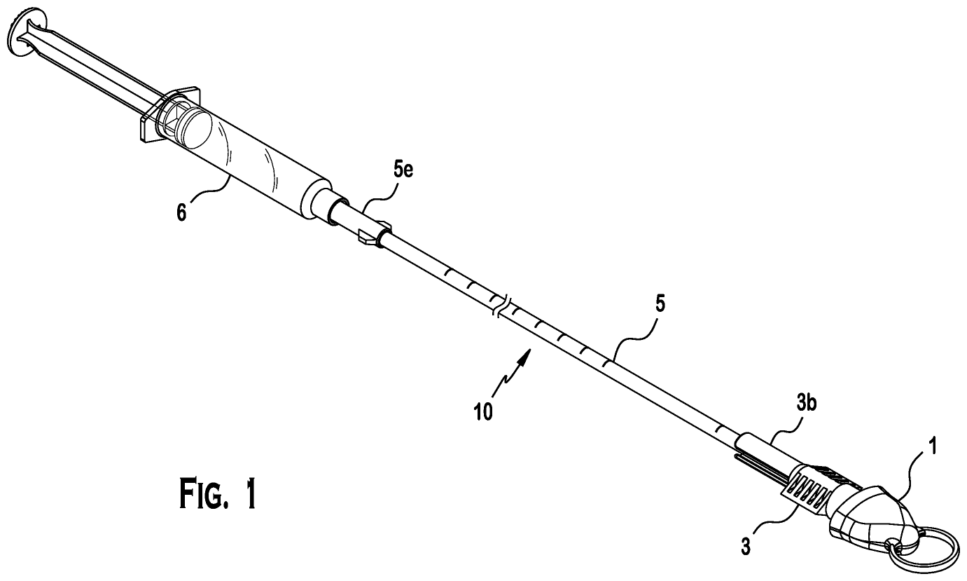

The FIGS. 1-8 establish the components and overall structure of a protective sheath assembly, according to the first embodiment of the invention.

As shown in FIGS. 1-4, the protective assembly 10 according to an embodiment of the invention, as a whole, is shown and generally includes a cap 1, an introducer tip 2, a grip housing 3, a retention device 4, and a graduated tubular style catheter/protective sheath assembly 5. The graduated tubular style catheter/protective sheath assembly 5 includes a catheter 9 having a proximal and distal end and is provided at the proximal end (the end away from the patient while in use) of the catheter with a connection or fitting, as will be discussed.

As shown in FIGS. 1-4, the cap 1 covers and protects the introducer tip 2, while also maintaining a lubricant jelly contained in system. The cap 1 is a plastic, such as polyvinyl chloride (PVC), but can be made from other known plastics. The cap 1 should be easily removed but should not pull the introducer tip 2 off of housing 3 when removed.

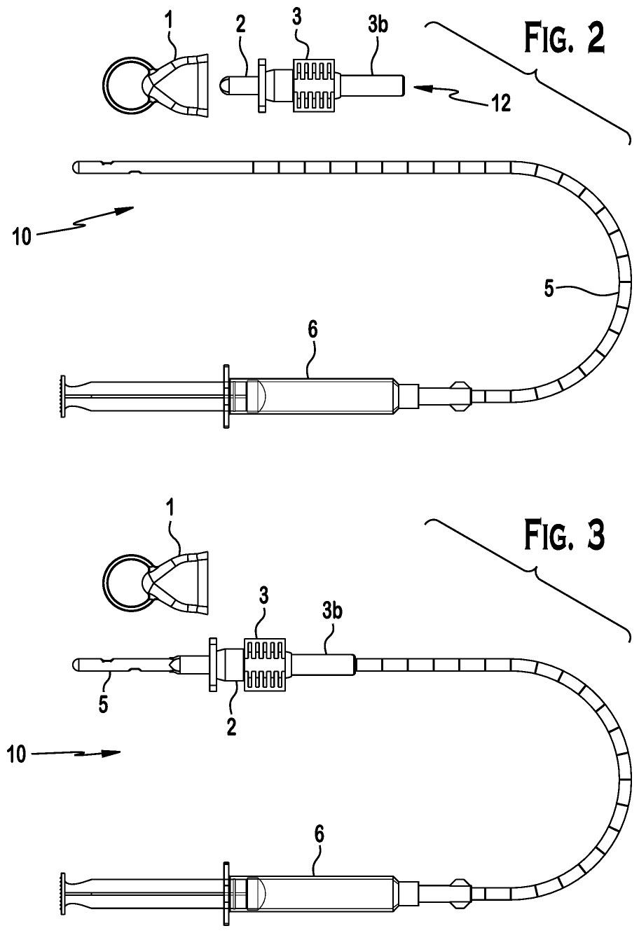

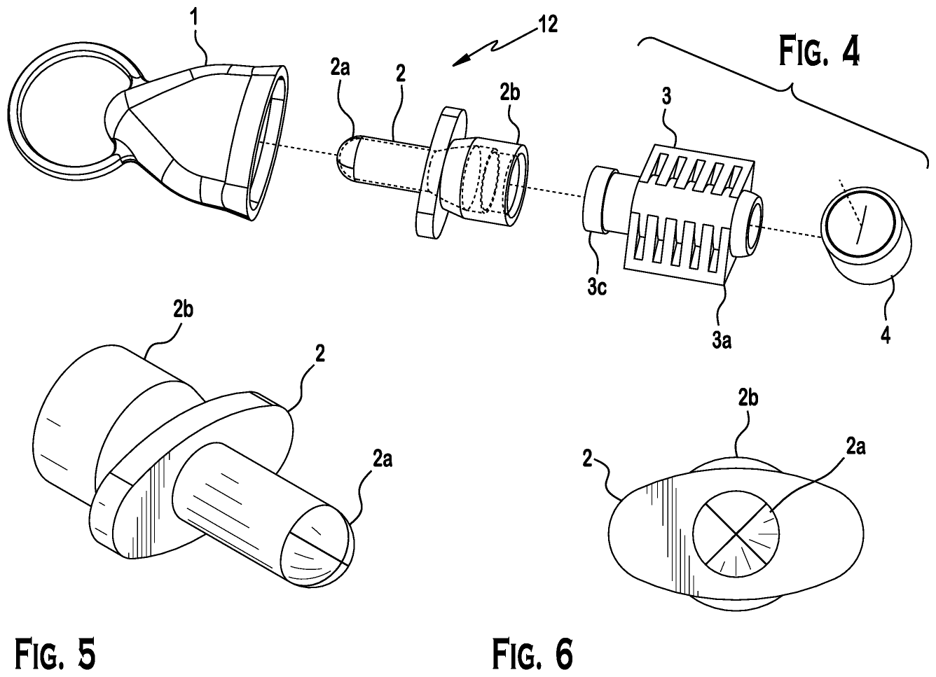

In the shown embodiment, the introducer tip 2 is made from a soft silicone, though it is contemplated that other soft, flexible material may be utilized instead, such as rubber or other flexible polymers. As shown, the introducer tip 2 is approximately 1.5 cm in length and the distal tip end 2a will have slit seal, which may be an “X” shaped cut, as can be seen with reference to FIGS. 5 and 6, in the distal tip end 2a to allow for the graduated tubular style catheter/protective sheath assembly 5 to pass there through, as depicted in FIG. 3. The introducer tip 2 should have a collar 2b to fit over and onto, or otherwise secure, the grip housing 3.

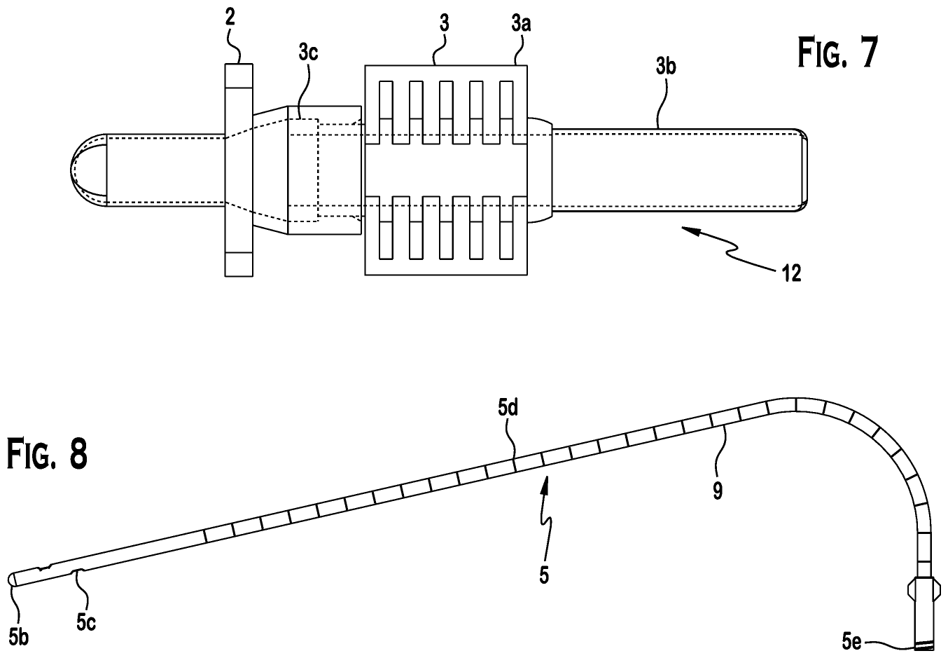

As shown in the detail view of FIG. 7, as an exemplary embodiment, the introducer tip 2 is rigidly secured to the grip housing 3 in any suitable fashion. As depicted here, the introducer tip 2 is secured to the grip housing 3 by sliding over a flanged or barbed end 3c of the grip housing 3, which serves to retain the introducer tip 2 in position relative to the grip/housing 3. It is contemplated that alternative securement techniques may be employed and would be familiar to those of skill in the art, including, but not limited to the use of clamps, adhesives, and welding techniques.

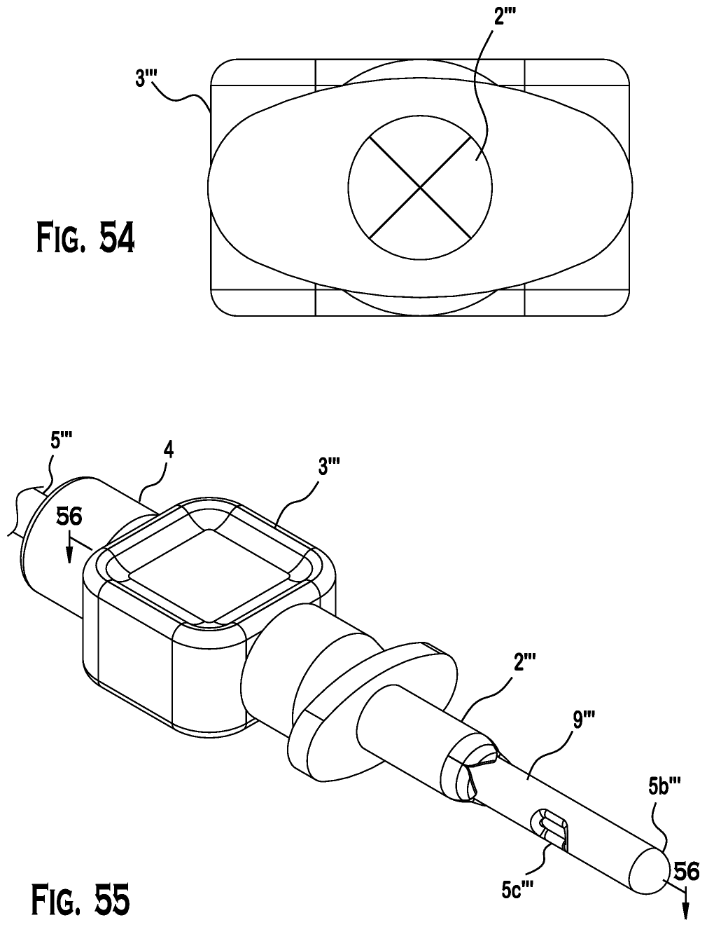

In the embodiment shown in FIG. 7, the grip/housing 3 is approximately 2.5 cm to 3 cm in length and includes a graduated tubular style catheter/protective sheath assembly receiving passageway 3b extending there through. In an embodiment, the grip housing 3 may include wing/flanges 3a on sides, or other surface features in order to grip between thumb and forefinger of the user. It is contemplated that alternative grip shapes may be provided and function similarly, for example, as can be seen with reference to FIGS. 53-55, as will be discussed. The inside diameter of the catheter/protective sheath assembly receiving passageway 3b through the grip/housing 3 should allow for a 10 fr catheter 9 to pass with ease.

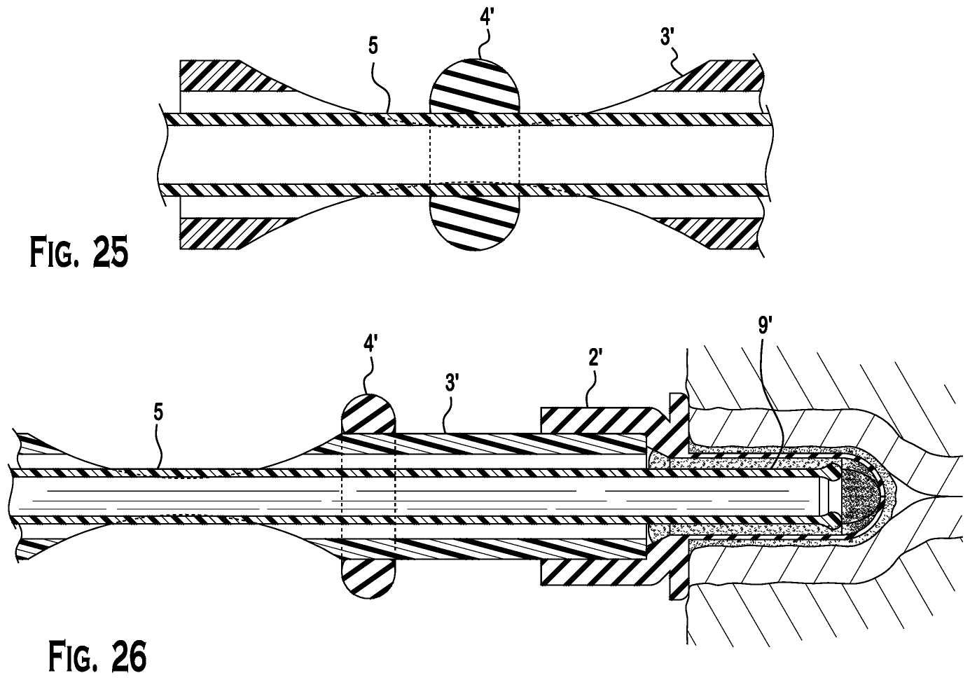

For the embodiments of a grip housing 3 that provide an enclosed volume, the interior volume may be filled with an amount of lube jelly to provide initial lubrication to the urethra upon insertion of the graduated tubular style catheter/protective sheathe assembly 5. In an embodiment, less than about 4, less than about 3, or less than about 2 milliliters of lube jelly are provided. In an embodiment, approximately 1-2 ml of lube jelly are provided. The base of the grip housing 3 will contain a retention device 4 to hold the graduated tubular style catheter/protective sheath assembly 5 in place and prevent lube jelly from coming out of the bottom of the grip housing 3. For embodiments of the grip housing 3 that do not feature an enclosed interior volume, as depicted with reference to FIGS. 27 and 42, it is contemplated that the anesthetic gel delivered via syringe may also serve as lubricant, as will be discussed.

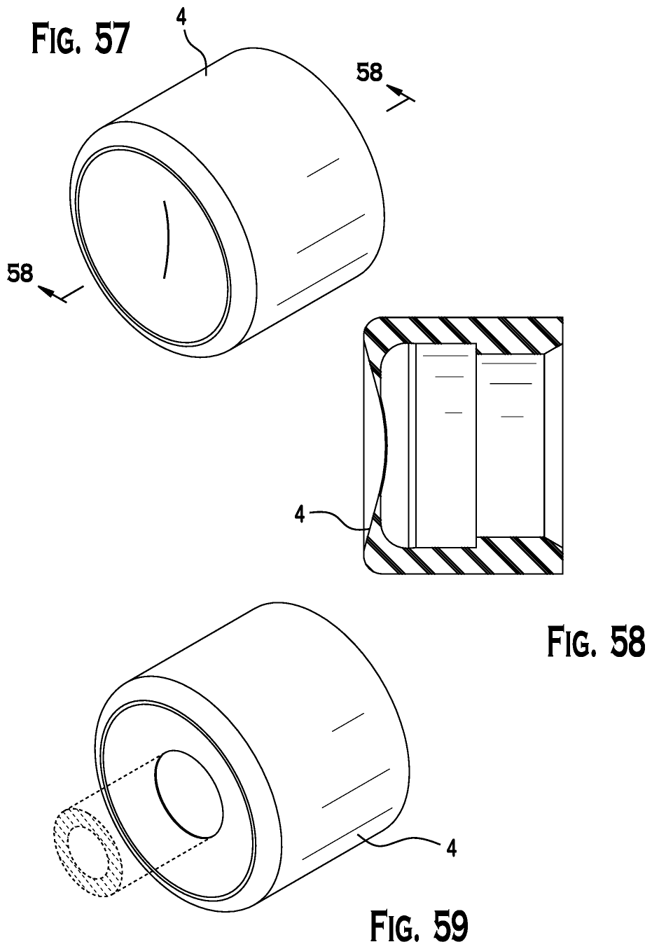

The retention device 4 may be any suitable material, such as silicone, or rubber, or other flexible polymeric material. The retention device 4 should fit over a small collar on the base of the grip housing 3 and be permanently attached so that it cannot fall off or become loose/lost during a procedure. The retention device 4, as depicted in FIG. 4, is provided with a slit valve in the face of the retention device, through which the graduated tubular style catheter/protective sheath assembly 5 may pass. Additional views of the retention device can be seen with reference to FIGS. 57-59. The slit valve is normally closed, as depicted in FIG. 57 to seal the lube jelly within the grip/housing 3, but is flexible and the slit can expand to accommodate the passage of the catheter 9 therethrough, as depicted in FIG. 59, while simultaneously applying a gripping force against the outer surface of the catheter 9, as the slit valve of the retention device 4 seeks to close and is urged by the elastomeric nature of the material against the catheter. The grip from such interaction provides enough friction that there is slight resistance, such that the free unintentional movement of the catheter is arrested, though the operator can easily overcome the resistance to allow for intentional movement of the catheter relative to the retention device 4 and grip housing 3, so as to allow advancement and retraction of the graduated tubular style catheter/protective sheath assembly 5, through the grip housing, as needed. It is contemplated that the slit of retention device 4 need not be limited to the single slit as depicted in FIG. 4, but may alternatively be provided with one or more slits to provide an “X”, “Y” or a star shaped opening, as would be familiar to those of skill in the art.

With reference to FIG. 8, there is shown the graduated tubular style catheter/protective sheath assembly 5, suitable for use in an embodiment of the invention. It is contemplated that the dimensions and features of the catheter 9 may be altered or adjusted as necessary for use in various procedures and various patients upon which the procedure is being performed. In the depicted embodiment, the catheter 9 should be 30 cm long +/−1 cm. However, it is conceived that in other exemplary embodiments, the catheter 9 can be of a length ranging from 5 cm to 80 cm long depending on the intended application of the invention. The graduated tubular style catheter/protective sheath assembly 5 provides a catheter 9, and a connection fitting at the proximal end. The catheter 9 may be any suitable form of catheter known to those of skill in the art and be suited for the various procedures described herein. All or a portion of the catheter 9 may be reinforced, such as may be provided with a braided catheter, to allow for a thinner wall thickness, while still providing adequate pushability and kink resistance. A reduced wall thickness of the catheter 9, allows for the inside diameter of the catheter 9 to be greater without increasing the outside dimension of the catheter 9, and can therefore accommodate a wider variety of devices therethrough, as will be discussed. The catheter 9 may be any suitable size, and may be in the size range of approximately 8 fr to approximately 36 fr, and may be, for example, 14 fr. In an embodiment where the catheter 9 is reinforced by having a wall construction with an internal braided component, the wall thickness may be 0.25 mm, and the catheter 9 may be provided with a lumen capable of accommodating a 4 mm device to pass therethrough.

In an embodiment, the distal end of the catheter/protective sheath assembly 10 has a catheter tip 5b at the distal end that should be open, polished, it may also have two opposing eyes 5c that are also polished and extend through the sidewall of the catheter 9.

In an embodiment, at least the distal-most portion (approximately 5 cm) of the catheter 9 may be reinforced by having an increased wall thickness relative to the balance of the catheter 9, alternatively, it is contemplated that the distal most portion of the catheter 9 may feature a different (e.g. stiffer) polymer composition than the balance of the guidewire; in this manner the distal most portion of the catheter 9 may be provided with greater resistance to bending than the balance of the catheter. In still another embodiment, the reinforcement of the distal tip 5b is provided by a rounded tip that is provides a greater thickness than the sidewall of the balance of the catheter 9. In an embodiment, the catheter 9 has at least a portion of the catheter length that features a braided or coiled reinforcing element within the wall thickness. In the embodiment having braided wall construction, there may be no need for further reinforcement at the distal portion of the catheter 9. The catheter 9 of the tubular style catheter/protective sheath assembly 10 shall have graduation markings 5d to guide the user on how far the graduated tubular style catheter/protective sheath assembly 10 is inserted in the body, for example, when directed into the urethra or nasal cavity.

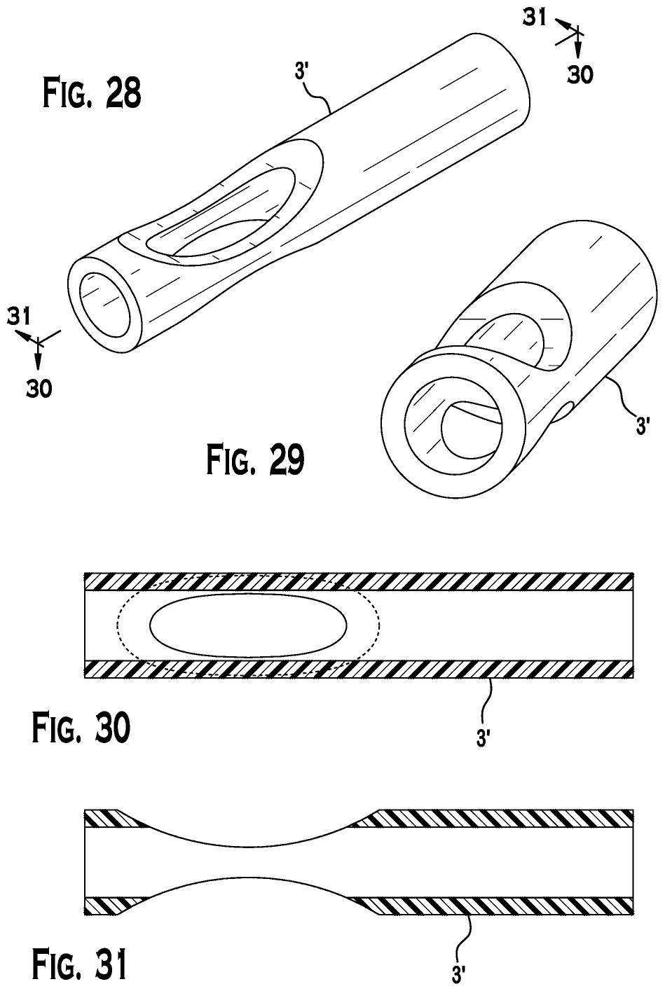

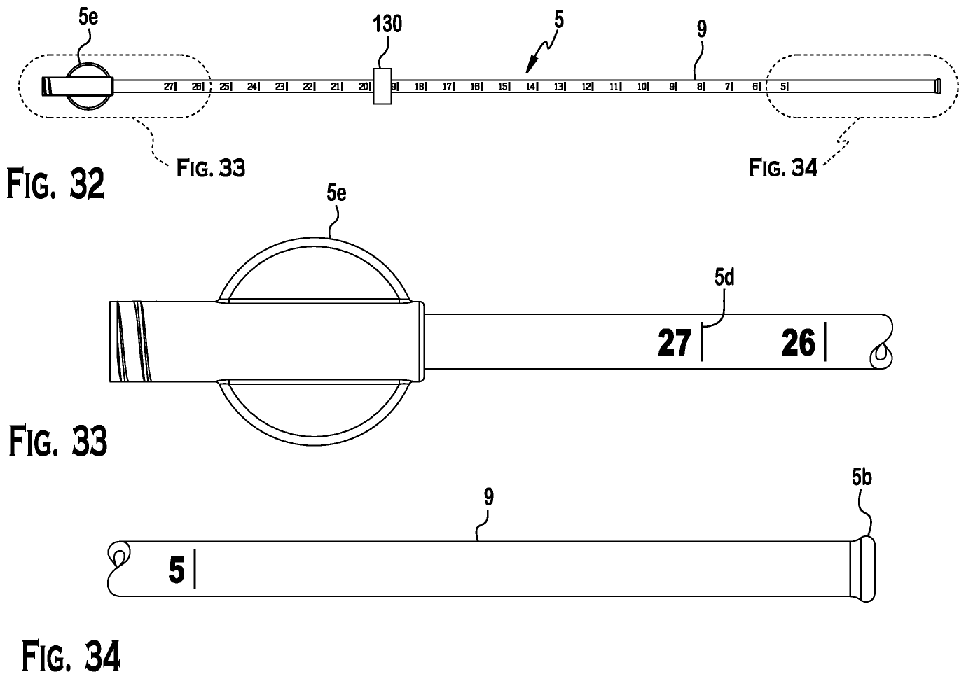

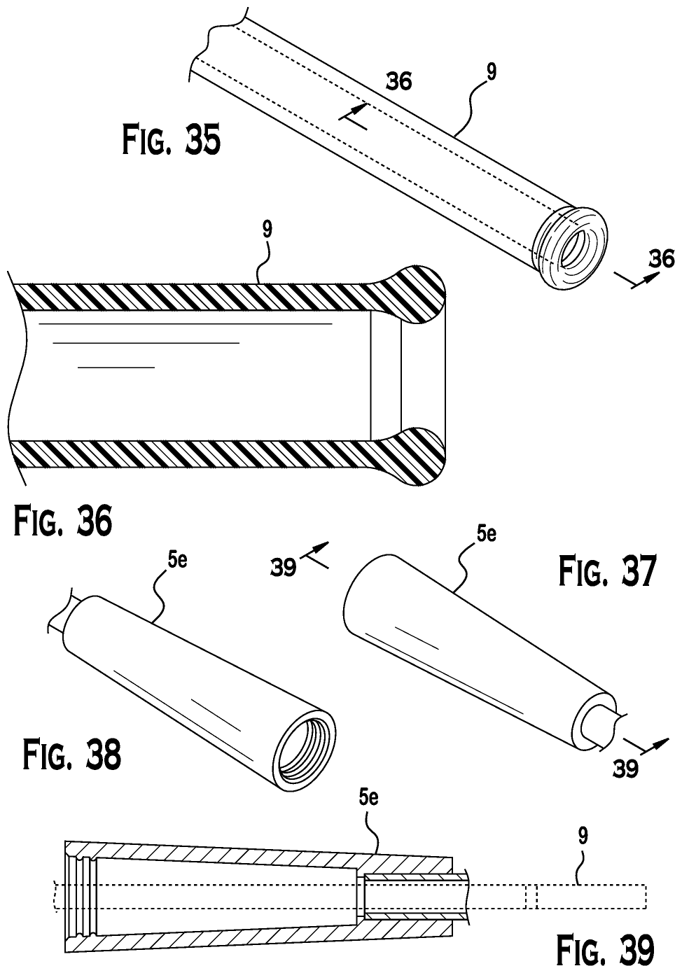

The “zero” reference will be located approximately 2.5 cm from the distal tip of the catheter 9 (this accounts for the length of the grip housing 3). Additional marks on the graduated tubular style catheter/protective sheath assembly 5 begin at 5 cm proximal from the zero reference point, and subsequently at 1 cm graduations and labelled to identify the actual measurement from the respective mark to the distal tip of the catheter, (i.e.,: 6 cm, 7 cm, 8 cm, 9 cm, etc.). In an embodiment, the catheter 9 may also have smaller sub-marks indicating every millimeter. It is recognized that the graduation marks may be placed differently, or adjusted for a different scale, as necessary to suit the user and/or the procedure. A representative embodiment of the catheter 9 can be seen with reference to FIGS. 32-36, with FIG. 33 depicting the proximal end of the embodiment of the catheter 9 having a luer connection, and FIG. 34 depicting the distal most portion of the catheter. FIG. 35 depicts a perspective view of the distal tip of the catheter 9. FIG. 36 depicts a cross-section view of the distal tip of the catheter 9.

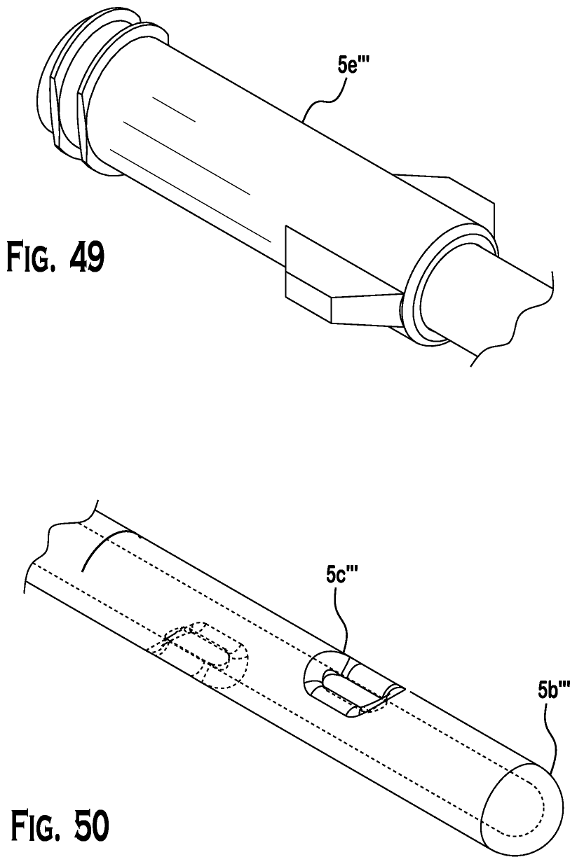

In an alternative embodiment of the catheter 9, as depicted in FIGS. 46-48, the distal tip 2 may be a rounded and closed tip, and feature one or more opposing eyes 5c in the sidewall of the catheter 9, as depicted in FIGS. 48 and 50.

In the embodiment shown in FIG. 1, the proximal end of the catheter 9 may have a connection or fitting. As depicted in FIG. 1, there is provided a luer lock connector 5e rigidly secured to the proximal end of the catheter 9. Any device having a complementary luer component, such as the syringe 6 having a female luer component may be secured to the proximal end of the catheter 9, as necessary. Rather than a luer lock connector, it is contemplated that at the proximal end of the catheter there may be provided a valve device, such as a one-way valve. For instance, in an exemplary embodiment, a one-way valve device may be provided to prevent back flow from the catheter 9 when a syringe 6 is removed from a connector such as a luer slip.

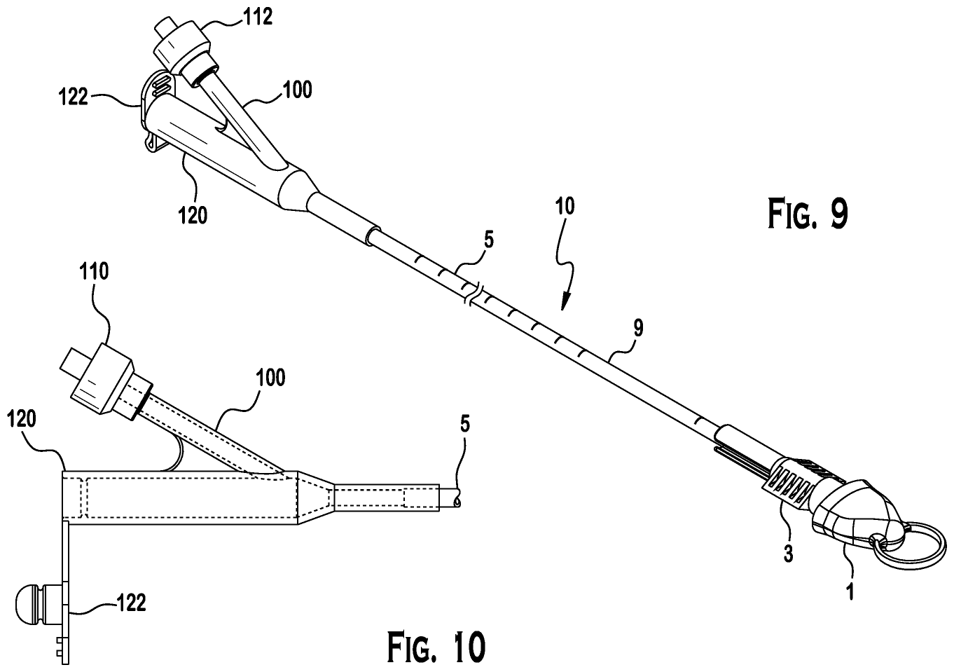

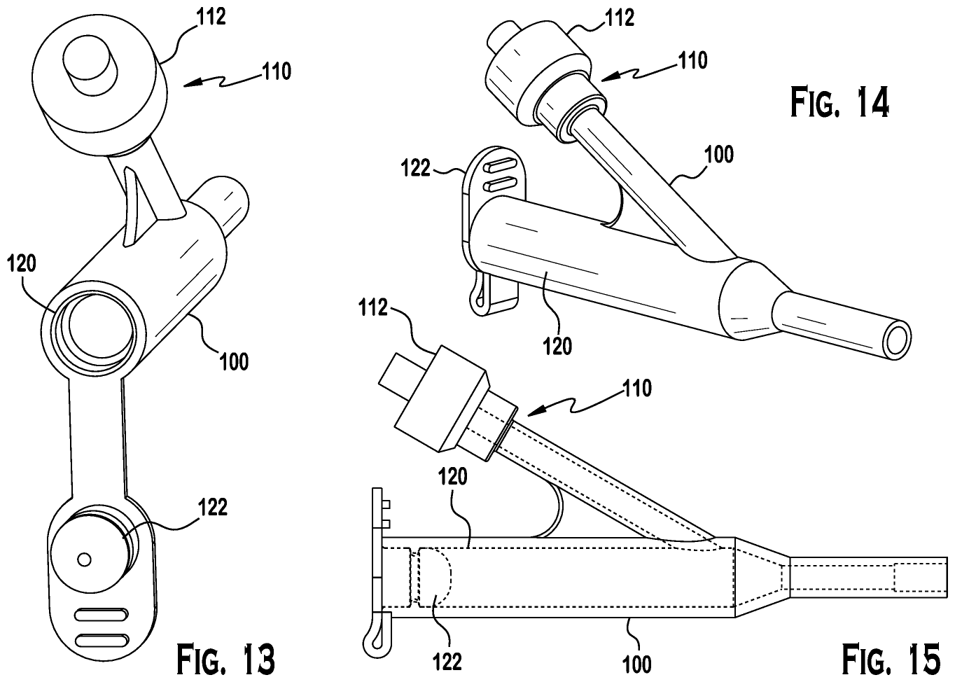

Alternatively, for any of the embodiments described herein, the connection at the proximal end of the catheter 9 may be provided as depicted in FIG. 9; as shown, the graduated tubular style catheter/protective sheath assembly 5 may have a “Y” type junction 100 that is rigidly secured to the catheter 9. An isolated “Y” type junction 100 is depicted in several views in FIGS. 10, 13-15, where the “Y” type junction incorporates a luer connection end 110, optionally provided with a one-way valve, to which may be removably attached a fluid filled syringe. Prior to attachment of the syringe 6, the luer connection 110 and optional one-way valve may optionally be sealed with a complementary luer cap 112 that can be removed to expose the luer connection 110 with optional one-way valve, for accepting the complementary luer fitting of a device to be secured to the catheter 9. In the embodiments where a syringe 6 is to be connected to the catheter 9, the syringe 6 may be used, for example, to dispense a medication or lubricant fluid, or alternatively to aspirate material or use suction to evacuate fluid out through the catheter 9.

With reference again to the “Y” type junction 100 of FIG. 9, the other branch of the “Y” connector 100 is a funnel type opening 120 that optionally incorporates a plug 122, where the funnel type opening 120 may remain sealed until it becomes necessary or desirable to unseal the funnel end opening, as may be necessary to allow fluid flow therethrough, for example, to drain excess urine from the bladder ahead of a procedure. Furthermore, the funnel type opening 120 may also allow for the introduction of materials or a variety of medical devices, as mentioned above. The aspects and details of the “Y” type junction 100 will be discussed below.

As shown in FIG. 9, the protective sheath assembly 10, according to an embodiment of the invention, may include a dual port “Y” connector 100 into one catheter 9. As shown in FIG. 15, the “Y” connector 100 would include a pair of ports. Each of the ports may be sized according to the planned use, and at least one may be provided with, for example, an internal dimension corresponding to a 14f diameter, and would easily integrate with the graduated tubular style catheter/protective sheath assembly 10 described above, and thus can be used as a dual-use catheter/sheath assembly 10. The Y connector 100 may provide a luer lock port 112, and a funnel type port 120. Alternatively, the Y connector 100 may provide a valve device, such as a one-way valve (not shown) as the first branch of the Y connector, and a funnel type port 120 as the second branch of the Y connector.

The embodiment of the protective sheath assembly 10 having a dual port connector 100 will accomplish various procedures. The protective sheath assembly 10 facilitates anesthetization of the entire urethra utilizing a 2% lidocaine solution, delivered through the catheter 9 using a luer lock connection 112, and optional one-way valve, at the first port, by which the syringe 6 may be secured to the Y connector 100. In an exemplary embodiment, the graduated tubular style catheter/protective sheath assembly 10 will provide an open-ended, buffed catheter 9, which may be reinforced, as discussed above. The lubrication and anesthetic solution delivered through the catheter would evenly lubricate and dispense anesthetic gel, such as the 2% lidocaine Jelly, from the tip of the urethra to the bladder neck, as the catheter 9 is advanced, in a manner similar to that described above. The “Y” connector 100 enables either port (112, 120) to flow without disturbance into the interior of the catheter/sheath 9. In the embodiment of the protective assembly 10 depicted in FIG. 9, the first port of the Y-connector 100 would have a male luer end 112, while the second port would have a funnel tip end 120, and associated plug 122 capable of reversibly sealing the funnel tip of the second port.

In an embodiment of the invention, the catheter 9 of the graduated tubular style catheter/protective sheath assembly 5 may be 15.5 cm (6 in)-40.5 cm (16″) with a mean length of 30.5 cm (12″) and have a diameter of 3.3 mm (10 FR)-6 mm (18 FR) with a mean diameter of 4.67 mm (14 FR).

For instance, in pediatrics, the catheter 9 may be approximately 25.4 cm (10″)-30.5 cm (12″) in length, and having any appropriate diameter, such as 5 FR, 6 FR, 8 FR & 10 FR diameter. For an adult male, the catheter 9 may be up to 16″ in length and having an appropriate diameter, such as 12 FR & 14 FR diameter; while for an adult female, the catheter embodiment may include a 15.2 cm (6″)-20.3 cm (8″) catheter 9 having an appropriate diameter, such as 12 FR & 14 FR diameter. For special applications, such as clearing debris or clots in the urinary tract (e.g., Haematuria), the catheter 9 may have up to a 40.6 cm (16″) length with an appropriate diameter, such as 16 FR, 18 FR, and 20 FR diameter. For bladder irrigation, the catheter 9 may be 40.6 cm (16″) long and have a 22 FR diameter. It is contemplated that the graduated tubular style catheter/protective sheath assembly 5 according to the invention may include diameters ranging from 2 FR-36 FR and length ranging from 7.6 cm (3″)-71.1 cm (28″).

Now, an exemplary description for a method of use for accessing a bladder using the protective sheath assembly 10 according to aforementioned embodiments of the invention will be described.

As previously mentioned, the graduated tubular style catheter/protective sheath assembly 5 of the protective assembly 10 may have graduated marks, such as 1 cm measurements beginning at 5 cm and going to 30 cm; this is unique to the invention and will allow the practitioner to note the actual depth that the graduated tubular style catheter/protective sheath assembly 5 is entered into the body.

The protective sheath assembly 10 is provided in a hermetically sealed package, the concept is that the practitioner opens the package and secures the graduated tubular style catheter/protective sheath assembly 5 of the protective assembly 10 to a syringe 6, which may be filled with an amount of medication, for example, an anesthetic gel, using the male luer lock for ease of use and sterility. Next, the user holds the penis upright on the sides so that the urethra is not pinched. If the penis is uncircumcised, simply retract the foreskin. Then, the user washes the glans penis from the urethral opening (tip) to the base of the glans with disinfecting scrub, water and/or a disposable disinfecting wipe(s). The user may repeat the washing two more times with a new disinfecting wipe each time. Next, while holding the penis upright with one hand at a 45-to-60-degree angle from the body, the user slowly inserts the introducer tip 2 into the (glans) penis. This is performed, for example, using the left-hand index finger and thumb to hold the bulbous of the penis, freeing the right hand. Then, the right hand is used to insert the introducer tip 2 into the urethra. Once introduced, the left index finger and third finger would grasp the ergonomic handle hold of the grip housing 3 in place to allow the right hand to be free to administer product through the catheter 9.

Then, the user may gently advance the catheter 9 of the protective assembly 10 through glans penis into the urethra slowly while dispensing a medication and/or an anesthetic gel (i.e., Lidocaine) by advancing the plunger of the syringe within the syringe body and proceeding until the distal tip of the catheter 9 meets resistance at the urethral sphincter. The user should not pull and/or push on the tube of the protective assembly 10 if and/or when resistance is met. The user should wait 10 seconds and apply gentle, but firm continuous pressure as well as dispensing the medication and/or anesthetic gel until the catheter 9 advances further toward the bladder. The anesthetic gel anesthetizes nerves in the region, and additionally serves as a lubricant and helps to advance the catheter 9 past the urethral sphincter, better known as the neck of the bladder, which is not an option with known products. Once the distal tip 5b of the catheter 9 is situated within the bladder, the practitioner may allow the catheter 9 to remain seeded for 5-7 minutes, allowing the anesthetic to take effect before beginning or continuing any procedure utilizing the catheter 9. Urine will naturally seek to drain from the protective assembly 10 as the anesthetic takes effect. Drainage may occur either by removal of the syringe at the proximal end of the catheter 9 (of FIG. 1), or by unsealing the plug 122 at the funnel opening 120 of the Y-connector 100 (of FIG. 9). If the catheter 9 is using a luer lock connection 110 and is provided with a one-way valve in the Y-connector 100, drainage is prevented by backflow through the luer connection, and allowed through the other branch when the plug 122 is removed. The user should have two (2) 4-ounce urine specimen cups nearby to prevent contamination of the surgical field. The procedure is largely similar for practice on a female, with the introduce tip 2 being directed into the urethral opening and advancing the catheter 9 towards the bladder in a similar fashion as previously described.

The various embodiments described herein are capable of providing localized anesthetic infusion of the entire urethra, and will reduce the discomfort of the patient, when passing a medical device, such as an endoscope, biopsy needle/punch, surgical fiber and/or culture swab, etc. The embodiments of the invention thus are designed to safely anesthetize the urethra, from the glans penis to the neck of the bladder. The bladder neck is a group of muscles that connect the bladder to the urethra, these muscles tighten to hold urine in the bladder, and relax to release it through the urethra.

Urinary problems occur when abnormalities block the bladder neck and prevent it from opening completely during urination and/or completely closing post urination. The various embodiments of a protective assembly 10 described herein reduces discomfort and facilitates the safe passage of a suitable device, such as an endoscope, a biopsy needle/punch, surgical fiber and/or culture swab, as non-limiting examples, through the bladder neck, post localized anesthesia. Having a protective sheath in place to protect the epithelium along the urinary tract when having to reinsert a device, such as an endoscope and/or a biopsy punch, reduces the opportunity to contaminate the surgical field, and thereby reduces the likelihood of an infection, and prevents irritation and pain attributable to the procedure. The protective assembly 10 having a dual port connector 100 addresses the problem improving patient comfort & outcomes.

For the various embodiments described herein, the protective assembly 10 is provided with a protective introducer tip 2, which aids the introduction of the catheter 9 into the tract. The introducer tip 2 may be any suitable material, such as silicone, rubber. The introducer tip 2, as can be seen with reference to FIG. 5, has a slit valve, such as may be created by one or more cuts in the tip; which can be seen, for example, in an “X” cut in the end 2a, through which the distal end and body of the catheter 9 may pass. In use, at least a portion of the length of the tip 2 is to be inserted into the penis, thereby enabling the catheter/sheath to safely pass through the glans penis where the majority of bacteria resides. The front end of the holding device assembly 12, comprising the components depicted in FIGS. 4 and 7, provides an introducer tip 2, grip housing 3, with a retention device 4, and may be protected by cap 1 until removed for use. The holding device assembly 12 enables the catheter 9 to enter into the urethra after the introducer tip 2 is positioned in the urethra and lubricates the catheter 9 as it advanced through the grip housing 3 and is exposed to the lubricating gel contained within the grip housing 3.

In another application, the catheter 9 may be approximately 27 cm long (e.g., +/−1 cm) and include graduation markings to guide healthcare professionals on how far the catheter is inserted in the urethra. The “zero” marking should be approximately 2.5 cm from the distal tip of the catheter 9 (this would account for the length of the grip housing 3). A first distance marking is made at 5 cm, and another marking at 10 cm, with additional marks every 1 cm from 10 cm to 18 cm. The proximal end of catheter 9 should have a “Y” type junction 100 to include a luer lock connector 112 attached, as well as a funnel tip 120 with an attached plug 122, to the proximal end of the catheter 9.

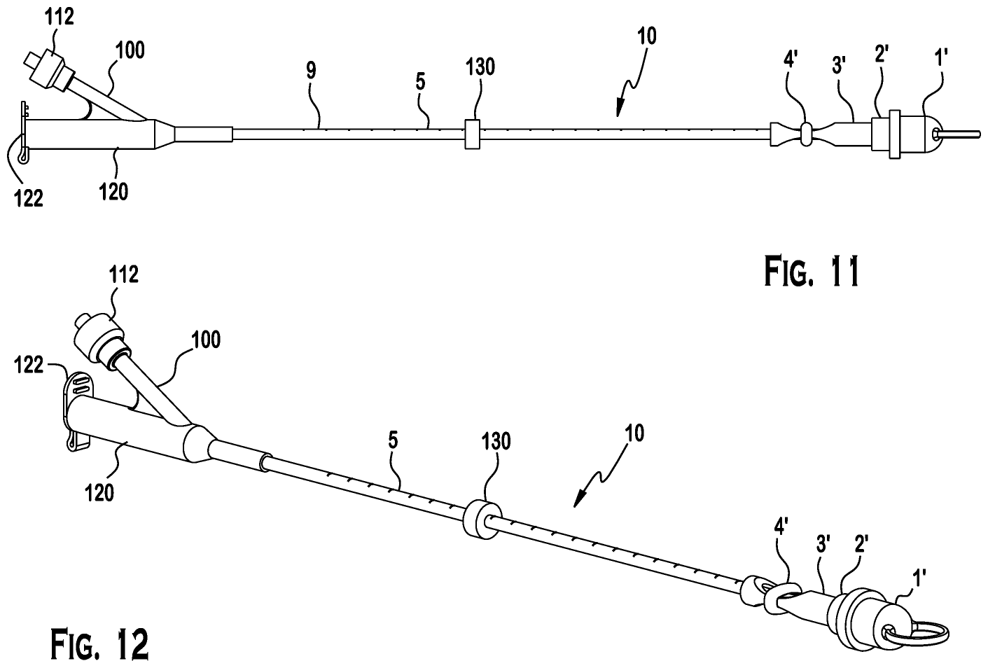

In an alternative embodiment, and as shown in FIGS. 11-12, the protective assembly 10 according to another embodiment of the invention, may include an integrated dual port connector 100 that communicates with the catheter 9. In this, or any of the embodiments described herein, there may be provided a ring or toroidal shaped marker 130, depicted in FIG. 12 as a “donut” shaped object positioned near the middle of the graduated tubular style catheter/protective sheath assembly 10. The marker 130 may be in the form of, for example, an elastomeric O-ring, or a polymeric material, that has an inside diameter that matches, or nearly matches the outside diameter of the catheter 9, such that the marker 130 can slide up and down the catheter 9, with slight friction against the catheter outside surface so as to maintain the position of the marker unless caused to move. Its physical purpose is to mark the depth that the graduated tubular style catheter/protective sheath assembly 5 is entered into the body. The marker 130 may be provided initially in a distal portion of the catheter 9, such that as the catheter is advanced into the body through the holding device assembly 12, the marker 130 is prevented from advancing as it encounters the grip housing 3, and slides in a proximal direction over the catheter 9 as it is further advanced into the body. Subsequently, as the catheter 9 is retracted proximally, the marker 130 would remain in relative position on the catheter 9, and with retraction of the catheter, the marker would be caused to move away from the grip housing 3, and thus can serve as an indicator of the penetration depth of the catheter 9 within the body during the procedure.

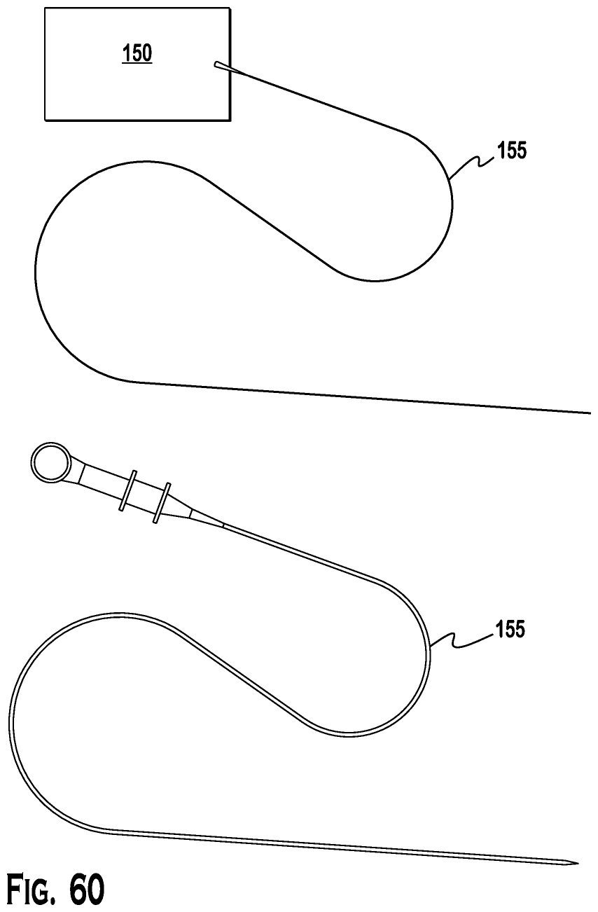

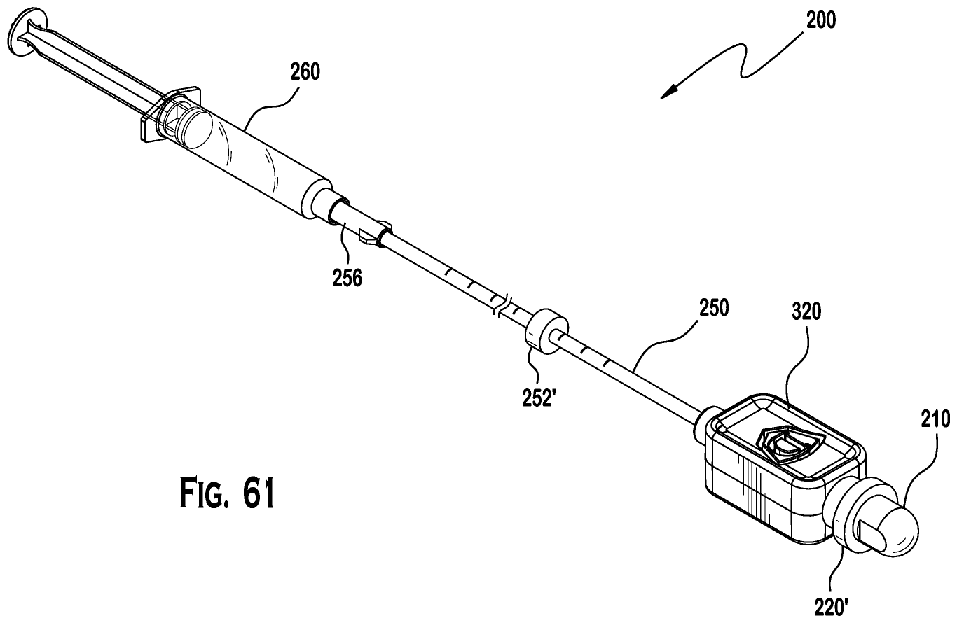

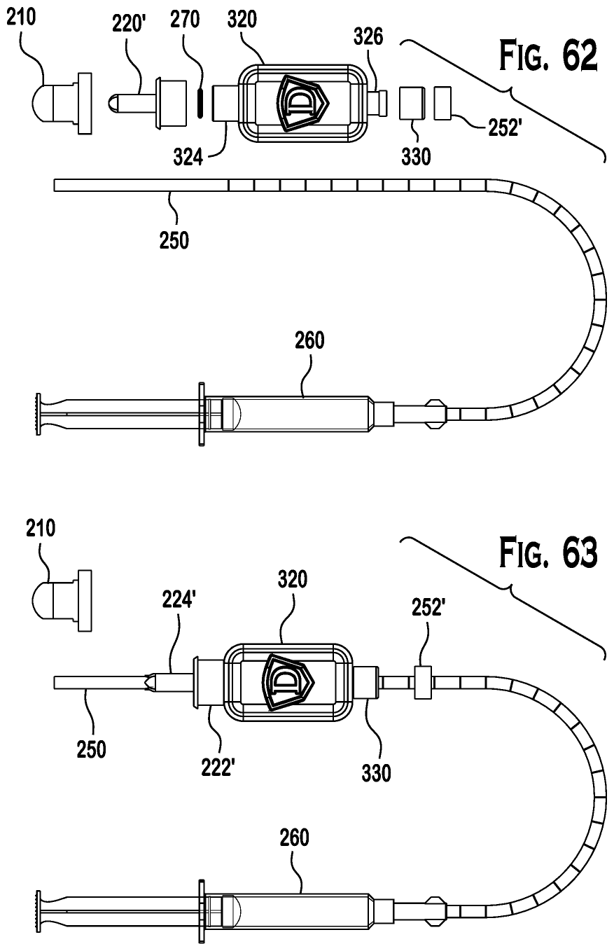

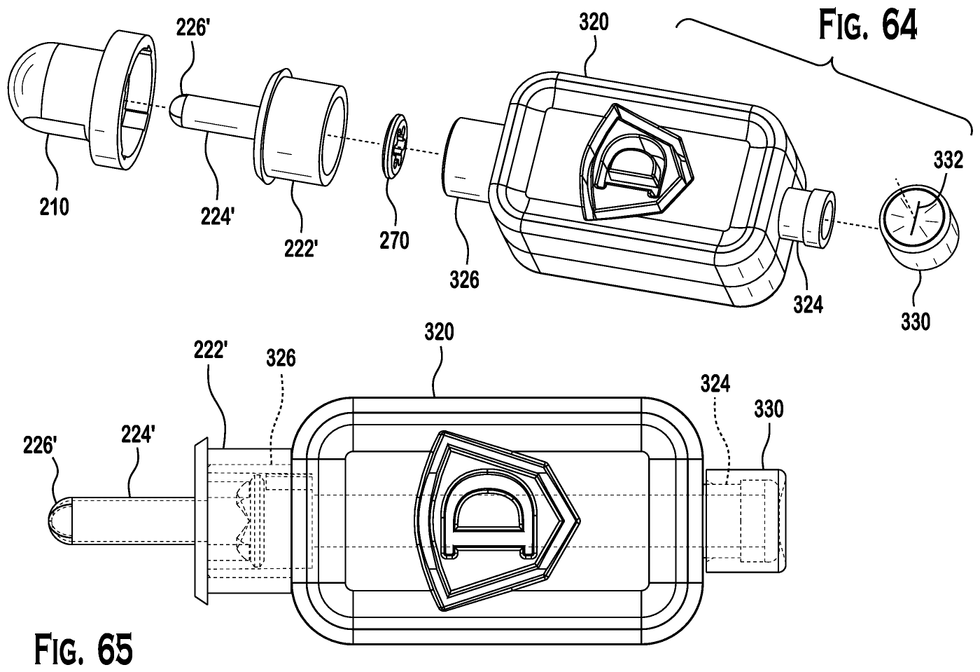

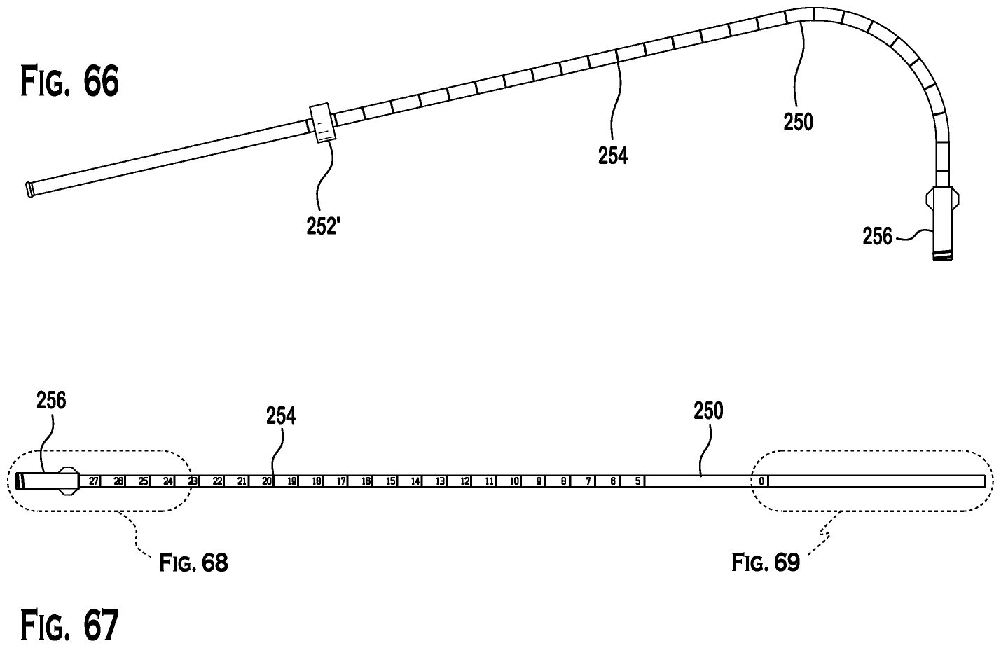

As discussed previously with regard to FIGS. 13-15, the dual port connector 100 includes a first entry section and a second entry section. The first entry section 120 is in parallel communication with the catheter 9 and includes a device receiving passageway and a plug 122 to seal the device receiving passageway. The aspect of this device provides two (2) services, first, once the entire urethra has been anesthetized, removal of the plug 122 will allow the bladder to be fully emptied in preparation of the procedure to be conducted, and second, then becomes the proximal access to the protective sheath assembly 10 of this device which allows the introduction and passage of any suitable device 155 of FIG. 60, for example, an endoscope, and/or a biopsy needle/punch, surgical fiber and/or culture swab to safely.

The second entry section 112, which may be either a luer lock as shown, or a one-way valve, of the dual port connector 100 is positioned adjacent to the first entry section and includes a fluid dispensing section that is in communication with the device receiving passageway of the first entry section 120 and catheter 9. It is contemplated that the second entry section 112 may be reversibly sealed, such as by providing a removable cap, or a valve device or other access system at the opening of the second entry section 112, such as a needle penetrable septum, slit valve, duckbill valve, one way valve, or a mechanical valve, such as Touhy-Borst adaptor, or any suitable valve as are known to be employed with catheter procedures, and would be familiar to those of skill in the art. The fluid dispensing section may be angled (i.e., 45 degrees) with respect to the device receiving passageway in the embodiment shown. The second entry section in the shown embodiment further includes a syringe adapter, and optional one-way valve. It is contemplated that when there is no syringe present on the second entry section, a removable cap suitable for mating to the luer connection may be provided in order to seal the opening, and thus may serve to prevent contamination and/or leakage through the opening.

The protective assembly 10 would be used as described above, however, the integrated dual port connector 100 facilitates multiple insertion of device and fluid into the tract, such as the urethra. In an exemplary use, medication may be dispensed to thoroughly irrigate and anesthetize the body passageway, and then accommodate devices such as endoscopes, surgical fibers, biopsy punch, culture swabs etc. for various procedures.

For instance, with any nasopharyngeal examination, multiple insertions subject the patient to significant discomfort as well as pathogenic bacteria that can induce a sinus infection. However, using the integrated dual port connector 100 according to the invention, medication can be dispensed to thoroughly anesthetize the nasopharyngeal cavity, and devices, such as endoscopes, surgical fibers, biopsy needles and/or punch, culture swabs, or other as will be familiar to those of skill in the art, can be introduced through the catheter 9 to work within a specific section.

In another exemplary method of use, the protective assembly 10 according to the invention can be used to deliver, by means of a surgical fiber, an ultraviolet wavelength that is lethal to microorganisms. In an embodiment of the invention, a user may use a short wave, low pressure, mercury vapor tube that can direct light via a device 155 that is a surgical fiber/flexible peripheral lead to the source of the infection through the catheter using the integrated dual port connector 100.

In an embodiment utilizing a surgical fiber routed through the protective assembly 10 to deliver a UV light for treatment within the body, the UV light source 150 of FIG. 60 utilized should produces a filtered short-wave UV and/or a non-filtered germicidal shortwave light with a range from UV 100 nm to 280 nm, that has a peak emission at 254 nm.

Ultraviolet light is part of the light spectrum, which is classified into three wavelength ranges:
UV-C from 100 nanometers (nm) to 280 nm
UV-B from 280 nanometers (nm) to 315 nm
UV-A from 315 nanometers (nm) to 400 nm

In an embodiment, the protective assembly 10 would further include an ergonomically designed hand piece that includes a rubber grip, whereby a surgical fiber/flexible peripheral fiber lead will slide into the grip of the light source 150, and thus transmits a UV-C light so that when the hand piece is turned on, will emit in the range of UV-C wavelength that is lethal to all microorganisms.

The UV-C light is transmitted via a device 155 that, in an embodiment, is a thin fiber-optic type, flexible peripheral lead, commonly referred to in the medical world as a surgical fiber found with lasers.

Before each use, the proximal end of a sterile, flexible peripheral fiber lead is inserted into the rubber grip on the hand piece, when the proximal end of the lead makes contact with, or is otherwise placed in a light transmitting relationship, such as by use of an optical connector, with the short wave, low pressure, mercury vapor tube inside the hand-piece, the flexible peripheral fiber lead will then transmit the specific wavelength of UV-C light through the catheter and onto the intended workspace. More specifically, when used with the embodiments of the protective assembly 10 described herein, the flexible peripheral fiber lead enters the body by means of the catheter 9 through the first entry section 120 and emits a wavelength band of UV light designed to kill pathogenic bacteria out the distal end of the surgical fiber. Approximately 95% of the ultraviolet energy emitted is at the mercury resonance line of 254 nanometers. The lumens of light delivered via the flexible peripheral fiber lead would be comparable to a strength already identified as proven to adequately kill pathogenic bacteria. Short wave, low pressure, mercury vapor tubes that produce ultraviolet wavelengths that are lethal to microorganisms, namely UV-C with a peak wavelength emitted at 254 nm, utilizing the concept used for wound healing/management via a surgical fiber used for laser surgery could also be construed as a fiber-optic lead.

In another embodiment, a hand piece may be provided that is configured to directly connect or communicate with the graduated tubular style catheter/protective sheath design 10 assembly.

The hand piece will be fully portable, ergonomically designed, and battery-operated UV lamp producing a shortwave UV light source. It is conceived that the rubber grip that holds the surgical fiber construed as a fiber-optic lead would be at a 90° angle to the hand-hold and a flexible peripheral lead would be inserted into the graduated tubular style catheter/protective sheath design assembly 5.

The light source 150 may be any suitable form or device capable of producing UV light with germicidal properties, and in an exemplary embodiment would be equipped with one (1) or two (2)-4-watt germicidal bulb producing namely a UV-C or UV-B shortwave UV light targeting a frequency range of 254 nm +/−100 nm. When the flexible peripheral lead makes contact with the shortwave UV light bulb, it will transmit a frequency of 254 nm through the flexible peripheral lead. In an embodiment of the invention, the device would be powered by any suitable energy supply and may be connected to an external source of power, or operate using a suitable configuration of on-board batteries, including AA, AAA, alkaline or 3 volt and/or lithium batteries to amply power the unit.

### Second Exemplary Embodiment

The FIGS. 11, 12, and 16-19 establish the overall structure of a protective sheath assembly according to the second embodiment of the invention.

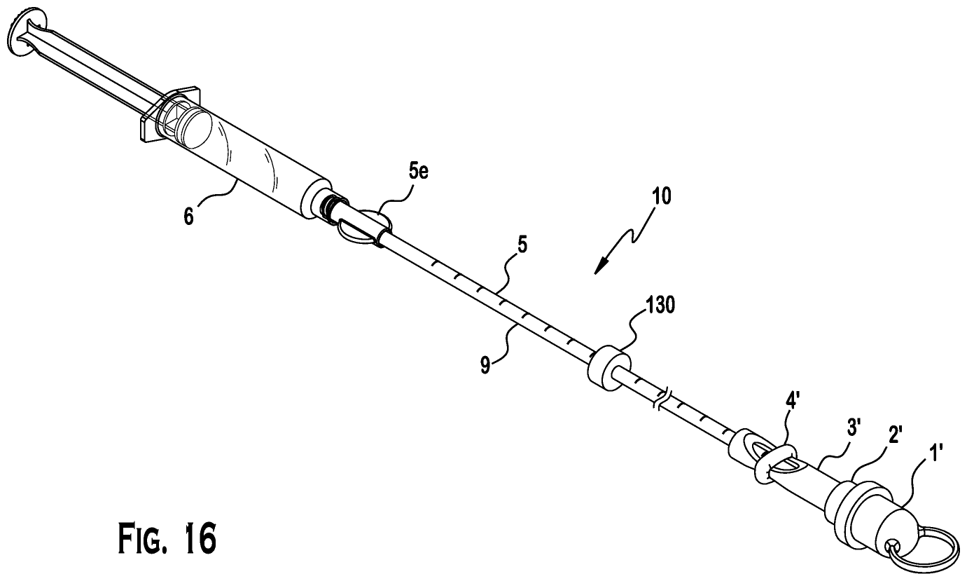

A protective assembly 10′ according to another embodiment of the invention, as a whole, is shown with reference to FIGS. 16-19, and generally includes a cap 1′, an introducer tip 2′, a housing 3′, a retention device 4′, and a catheter 9′. The catheter 9′ and any connection or fitting at the proximal end of the catheter 9′ form the graduated tubular style catheter/protective sheath assembly 5′ and may be advanced or retracted through the holding device assembly 12′ comprising the introducer tip 2′, housing 3′, and retention device 4′.

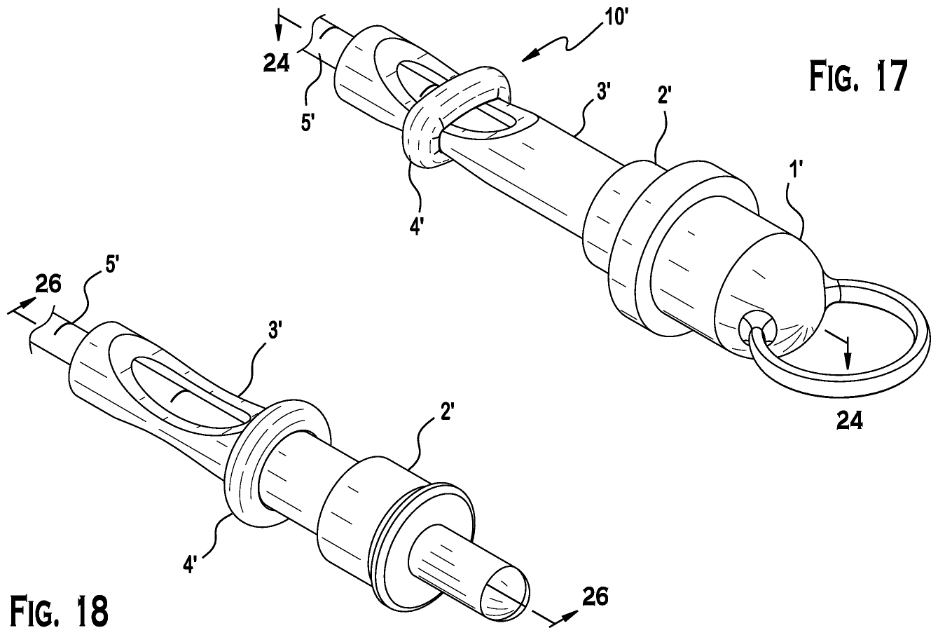

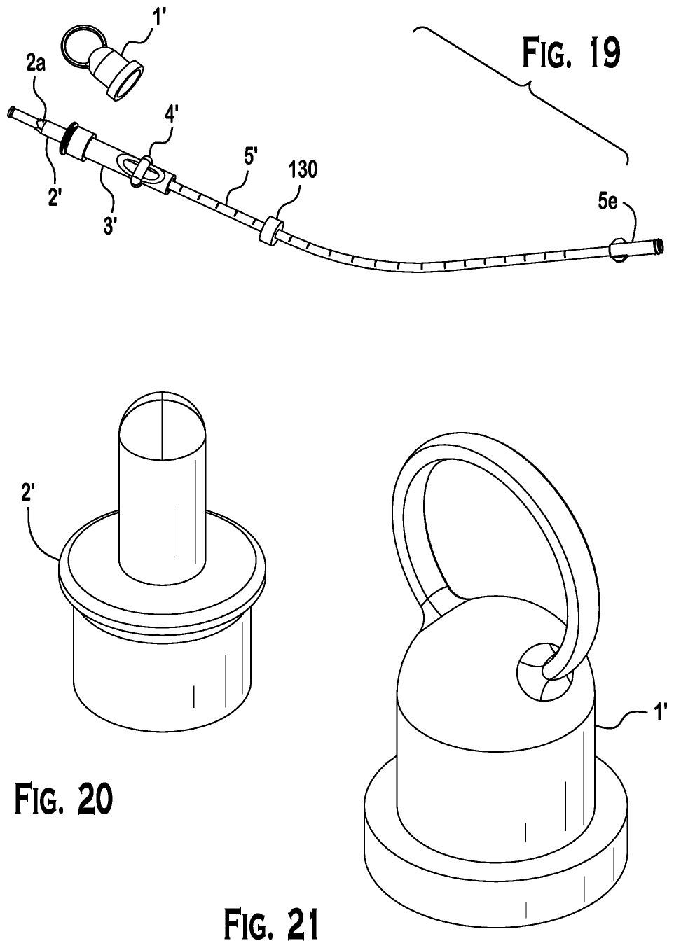

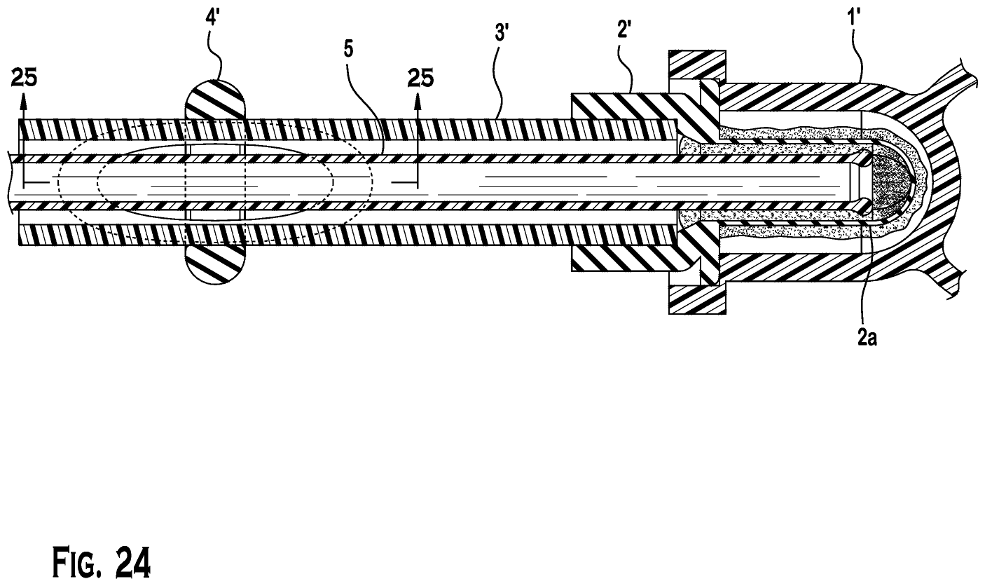

As shown in FIGS. 18 and 19, the graduated tubular style catheter/protective sheath design assembly 5′ of the second embodiment includes an introducer tip 2′, with one or more slits cut into the tip 2a′ to allow the catheter 5′ to pass freely there through. The introducer tip may be a silicone material, rubber, or other suitable elastomeric material. An expanded view of the introducer tip 2′ can be seen with reference to FIG. 20. The one or more slits in the tip 2a′ may be in the form of a star cut by providing 3 or more slices into the tip, such that when viewed end on, there is a star pattern, where the flexibility of the material allows each of the portions between adjacent slit sections to conform to the outside perimeter of the catheter passing through the tip. It is further contemplated that alternative slit configurations to the tip may be provided to allow the passage of the catheter therethrough, such as the “X” cut (2 slices), “Y” cut (3 radial slices), or a single slit that can accommodate passage of the catheter. The introducer tip 2′ was designed to be the first level of protection when the graduated tubular style catheter/protective sheath design assembly 10′ enters the glans penis/urinary meatus reducing the risk of transferring microbes further into the urethra. The protective assembly 10′ of the second embodiment may be fitted with a cap 1′ to protect the introducer tip 2′, as shown in FIG. 17. An expanded view of the cap 1′ can be seen with reference to FIG. 21. The cap 1′ as represented offers an easy pull cap design, that can be separated from the tip 2′ with a small pulling force, without dislodging the introducer tip 2′ from the housing 3′. A cross-section view of the protective sheath assembly 10′ can be seen with reference to FIG. 24. As discussed previously, an amount of lubricant jelly may be provided within the cap 1′ and against the distal end 2a′ of the tip 2′, leaving a coating of lubricant jelly on the introducer tip 2′ to facilitate introduction into the tract of a body, such as the urethra.

At least a portion of the graduated tubular style catheter/protective sheath design assembly 5′ of the second embodiment is reinforced to prevent kinking and/or snagging when passing the length of the urethra and especially when entering the neck of the bladder. The reinforced portion at the distal end of the graduated tubular style catheter/protective sheath design assembly may have one or more of: an increased wall thickness; stiffer polymer compositions, or reinforcing jacket, or all or at least a portion of the catheter 9′ may include a braided reinforcement as will be familiar to those of skill in the art. As mentioned above, the braided catheter may allow for a thinner wall thickness, with adequate pushability and kink resistance, yet still provide an internal diameter within the catheter 9′ to accommodate the various sizes of medical device 155 (of FIG. 60) that may be directed therethrough. The braided catheter and the benefits described may be usefully employed in any of the various embodiments described herein.

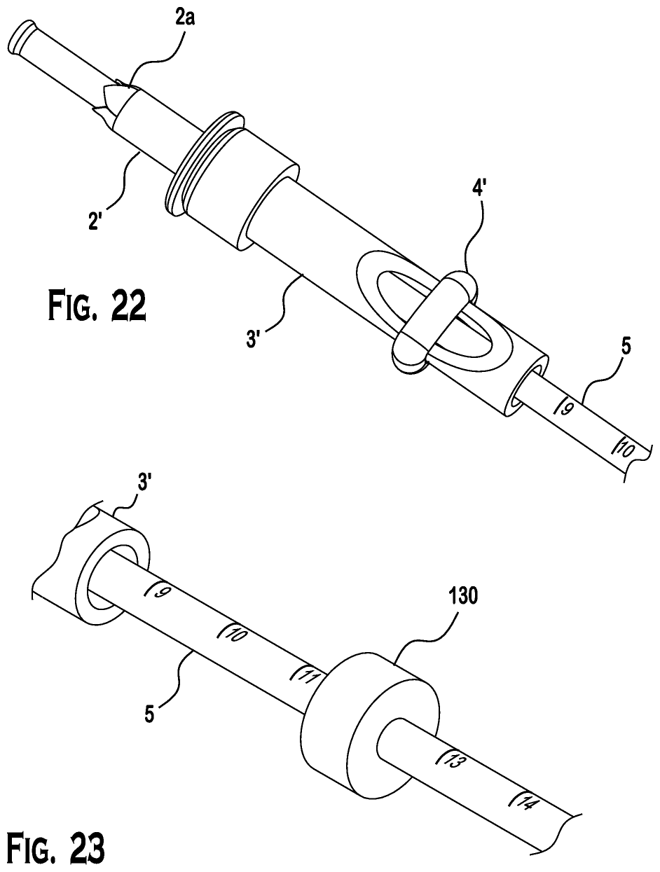

As shown in FIGS. 19 and 22, the introducer tip 2′ of the protective sheath assembly 10′ of the second embodiment includes any suitable form of a slit seal, depicted here as an “X” shaped cut into the tip 2a′, allowing the catheter 9′ of the protective assembly 10′ to pass freely, although, prior to the catheter 9′ passing through the opening of the introducer tip, the slit seal of the tip 2a′, shown here as the “X” shaped cut, would remain closed (as depicted in FIG. 20), thereby reduces the opportunity of contamination as the slit seal partially seals and protects the protective assembly 10′.

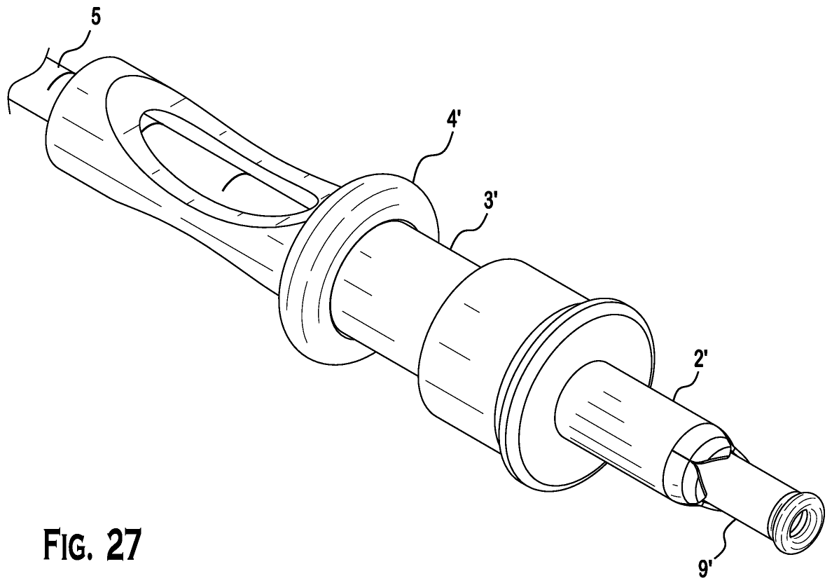

As depicted in FIG. 18, the protective assembly 10′ of the second embodiment includes a housing 3′ having tapered sides that expose the catheter 9′ tube making the dexterity of the device much easier for the practitioner to hold. The handhold of the grip housing 3′ can be fitted to accommodate any tube diameter so as to meet the needs of the procedure, i.e.: anesthetize the urethra, insert a scope, biopsy needle and/or medication. An isolated portion of the grip housing 3′ featuring the tapered sides can be seen with reference to FIGS. 28-31.

With reference to FIG. 22, the protective assembly 10′ of the second embodiment further includes a retention device 4′, such as an elastic band or O-ring, which is designed to resiliently fit over the housing 3′, and apply inward pressure to the tapered sides of the housing 3′, so as to apply drag/friction to the outer surface of the catheter 9′ as it passes through the grip housing 3′, thereby reducing backing out/slip, as shown in FIGS. 17 and 22. It is further contemplated that, when in position depicted in FIG. 17, the direct contact between the retention device 4′ gripping the catheter 9′ may provide the friction to prevent free movement of the catheter, which again can be overcome by the practitioner, thereby allowing for precise control of the catheter 9′. As depicted in FIG. 18, and in cross-section in FIG. 26, the retention device 4′ may be moved away from the tapered sides of the housing 3′, to an area of the housing 3′ that does not feature the tapered sides, but instead provides a full tubular structure, thereby relieving the inward pressure on the tapered sides and reducing the friction between the housing 3′ and the catheter 9′ outer surface. In this manner, the user may advance or retract the catheter 9′ device relative to the housing 3′, without any resistance to movement by the housing 3′ being urged against the catheter 9′. Once in, or approaching the desired position, the retention device 4′ may be moved back into the position depicted in FIG. 17, and the friction serving to prevent unwanted or unintended movement of the catheter 9′ relative to the housing 3′. The amount of friction with the retention device 4′ urging the tapered sides of the housing 3′ inward, as shown in FIG. 17, is not so great that all movement of the catheter 9′ relative to the housing 3′ is prevented, rather, the drag is such that the user is able to overcome the friction deliberately, yet will prevent unintentional movement. The drag by the retention device 4′ squeezing the tapered sides towards the catheter as described will thus enable precise placement of the catheter 9′ of the protective assembly 10′ of the second embodiment to the demands of the practitioner.

The catheter 9′, is similar to that described previously, in that it provides a tubular structure with graduation markings, as depicted in FIG. 23, which may be in any suitable increments, so that the practitioner can insert the protective assembly 10′ of the second embodiment with precision and the distance the catheter 9′ is advanced into the body can easily be easily recorded and/or monitored, for example, when using an external measuring device such as a C-scope, Ultrasound and/or X-ray.

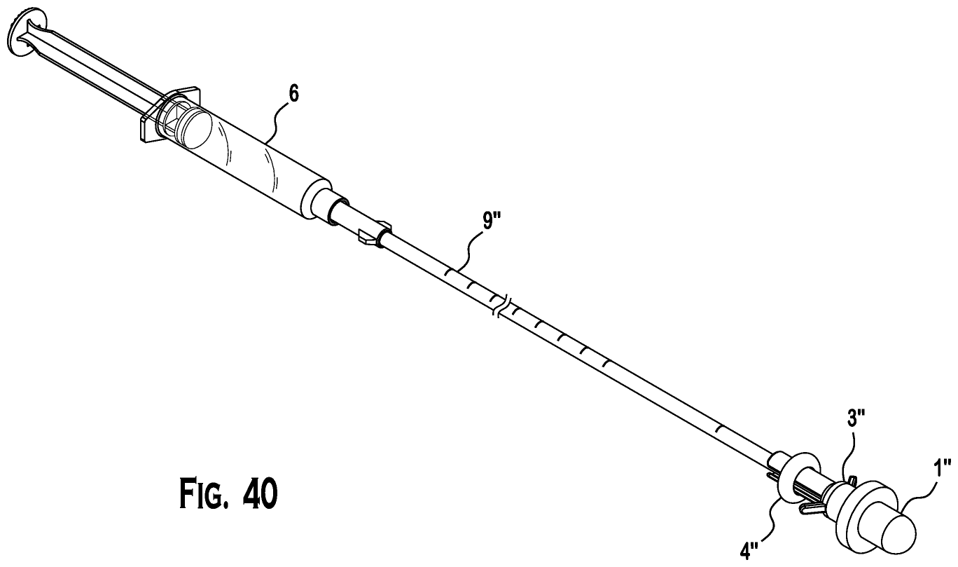

As shown in FIG. 23, the protective sheath assembly 10′ of the second embodiment further includes a marker 130, similar to that described previously. Such a marker 130 may be a resilient donut member (i.e., white) that provides a said non-slip “stop” that allows the measurement of the distance the catheter 9′ is advanced into the body to be recorded and/or future placement of the tube ahead of future examinations. The graduated tubular style catheter/protective sheath assembly 5′ of the second embodiment further includes a connection or fitting at the proximal end, such as a male luer lock, or valve. As depicted in FIG. 16, there is provided a luer lock rigidly attached to the proximal end of the catheter 9′. Alternative syringe securing mechanisms may be provided, such as by providing a catheter tip to accommodate a luer slip syringe, as will be familiar to those of skill in the art. An example of the luer slip connection which may be provided as an alternative to the luer lock component, can be seen with reference to FIG. 37-39. Alternatively, the graduated tubular style catheter/protective sheath assembly 5′ of the second embodiment may be provided with a Y connector 100, as has been described previously, as shown in FIGS. 11 and 12.

The use and various features of a protective sheath assembly 10′ according to a second embodiment of the invention will be as discussed previously, however, the accommodation being that the practitioner may reversibly move the retention device 4′ away from, or return to, the region of the grip housing 3′ featuring the tapered sides, in order to selectively remove frictional restraint, or apply frictional restraint, against the movement of the catheter 9′, as needed. Free movement of the catheter 9′ is allowed when the retention device 4′ is in the position depicted in FIGS. 26 and 27. Overcomeable frictional resistance is provided when the retention device 4′ is in the position depicted in FIGS. 22 and 24, with the retention device 4′ placed over the tapered sides of the catheter 9′.

### Third Exemplary Embodiment

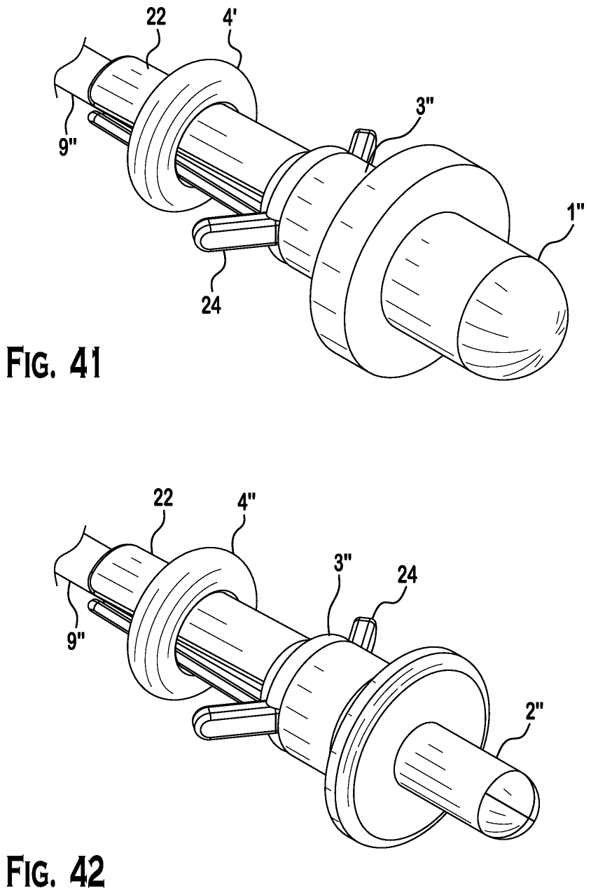

The FIGS. 40-45 establish the overall structure of a protective sheath assembly according to a third embodiment of the invention.

With reference to FIGS. 40 and 42, a protective sheath assembly according to another embodiment of the invention, as a whole, is shown and generally includes a cap 1″, an introducer tip 2″, a housing 3″, a retention device 4″, a catheter 9″, and a syringe 6. With the exception of the retention device 4″ and housing 3″, the remaining components of the third embodiment are substantially similar to the respective components already described.

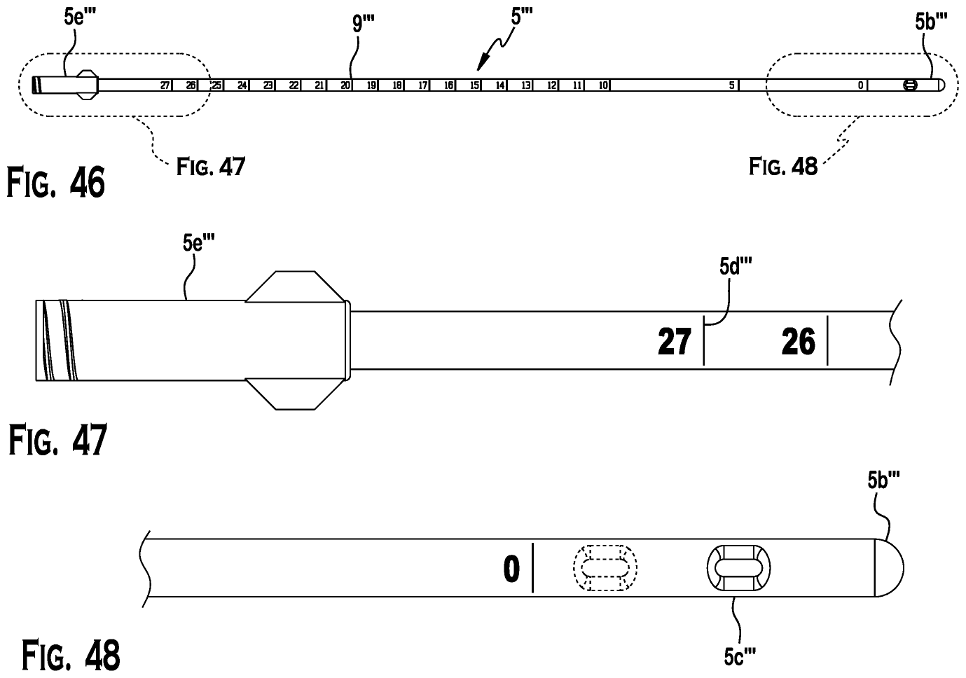

With reference to FIG. 40, there is depicted a third embodiment of the grip housing 3″, having a bifurcated tubular housing, as depicted in expanded view in FIG. 42, and providing any suitable connection fitting as has been described previously, such as the Y-connector, or a single luer component as shown at the proximal end of the catheter 9″, to which may be secured a syringe 6. The grip housing 3″ (shown in isolated view in FIG. 44 and in section view in FIG. 45) provides a pair of gripping surfaces 22 that can extend on either side of the catheter 9″ as it is directed through the housing 3″, and further provide a pair of extended wings 24 that protrude laterally away from the catheter 9″. The housing 3″ provides surfaces against which the practitioner may grasp to advance or retract the grip housing 3″.

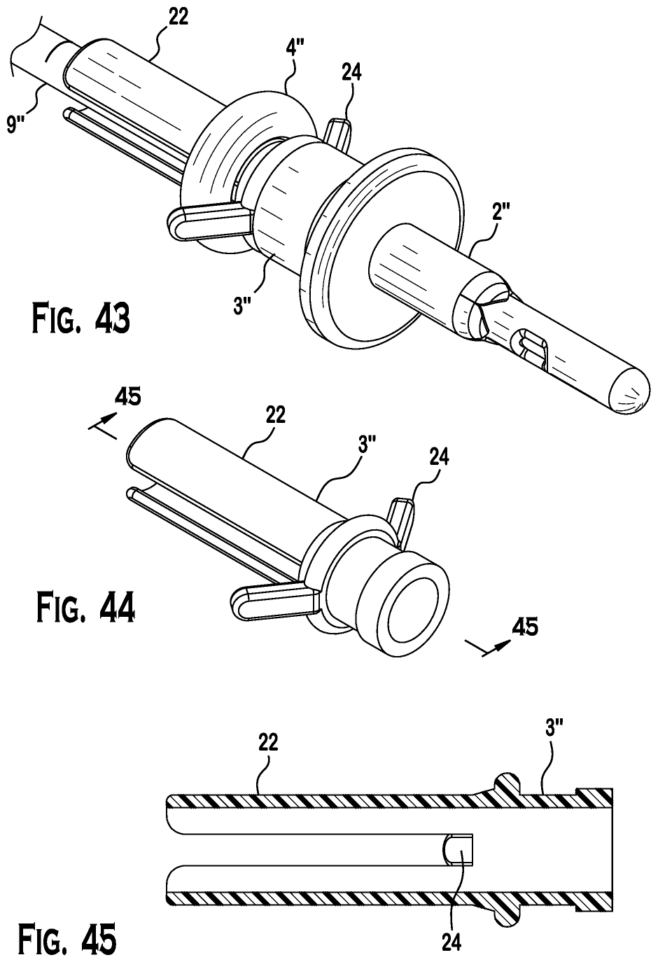

In the embodiment of FIG. 40, there is provided a retention device 4″ which may be in the form of an elastomeric ring or toroid, as described previously, and as deployed in FIG. 40, can be seen to apply pressure against the gripping surfaces 22 to secure or prevent unwanted movement of the catheter 9″, relative to the housing 3″. The amount of grip applied by the gipping surfaces 22 can be controlled by adjusting the placement of the retention device 4″, as can be seen with reference to FIGS. 42 and 43, thereby adjusting the amount of leverage the retention device 4″ has in applying a compression force to the gripping surfaces 22, in turn adjusting the amount of friction between the catheter 9″ and the gripping surfaces 22. For example, moving the retention device 4″ distally towards the tip 2″ tends to reduce the friction (as shown in FIG. 43) to allow free movement of the catheter 9″ relative to the grip 3″, while placing the retention device 4″ in a more proximal position (as depicted in FIG. 42) and towards the free ends of the gripping surfaces 22 tends to increase the friction, and serves to prevent unintended free movement of the catheter 9″ relative to the grip 3″. As before, the amount of friction is such that unintended movement of the catheter 9″ is prevented but can be overcome by the practitioner to allow precise placement of the catheter 9″.

### Description of a Fourth Embodiment

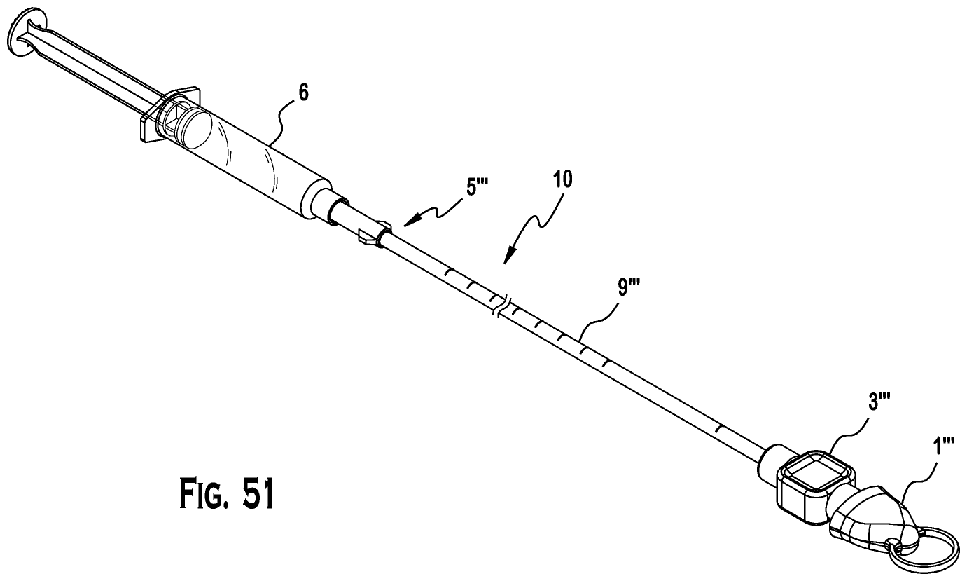

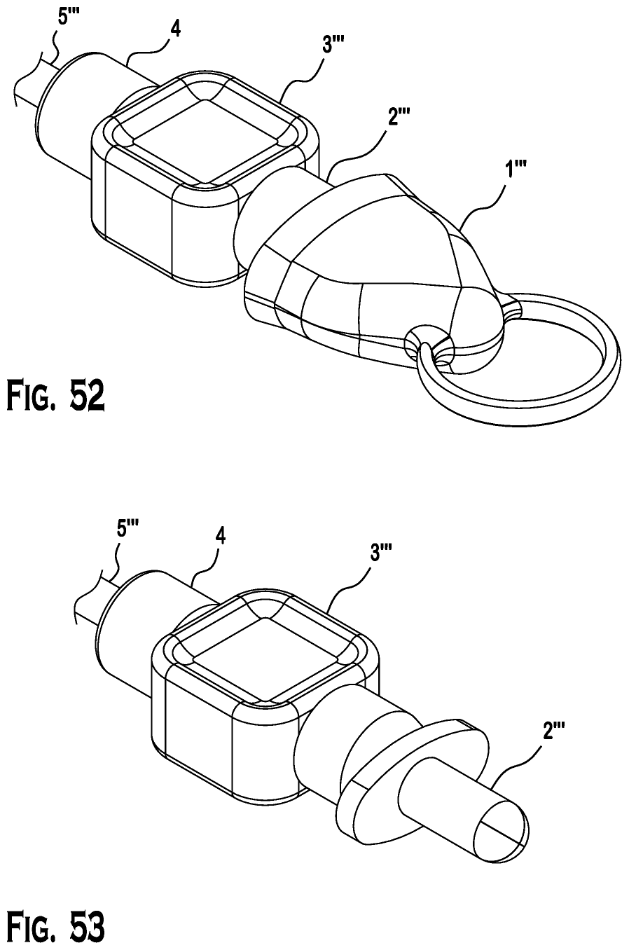

The FIGS. 51-55 establish the overall structure of a protective sheath assembly according to a fourth embodiment of the invention.

With reference to FIGS. 51 and 53, a protective sheath assembly according to another embodiment of the invention, as a whole, is shown and generally includes a cap 1″′, an introducer tip 2″′, a housing 3″′, a catheter 9″′, and a syringe 6. With the exception of the housing 3″′, the remaining components of the fourth embodiment are substantially similar to the respective components already described.

As shown, there is depicted an embodiment having a generally cuboid or rectangular prism shaped grip housing 3″′ and having a luer component at the proximal end of the catheter 9″′, to which is secured a syringe 6. It is contemplated that alternative connections at the proximal end of the catheter 9″′ may be provided, such as the Y-connector previously described. The housing 3″′ is generally prism shaped to provide gripping surfaces that provide large surface area that can comfortably be pinched between the practitioners thumb and fingers to securely hold the grip when manipulating a portion of the protective assembly 10. As depicted in FIG. 53, the gripping surface maybe slightly indented to provide additional security when gripping and further may prevent fatigue or slipping off when holding the housing 3′, as there would be a slight lip protruding above the gripping surface about the perimeter of the gipping surface that would tend to minimize the potential for slipping. This indented gripping surface feature may be provided on one or both of the gripping surfaces.

As with the first embodiment, the housing 3′ may contain an amount of lubricant jelly, that is contained within the internal volume of the housing 3′ by the tip 2″′ at the distal end of the housing 3″′, and at the proximal end of the housing 3′ by a retention device 4, as discussed above with reference to FIGS. 57-59. The catheter 9′ as it is advanced through the housing, first passes through the retention device 4, passes through an amount of lubricating jelly, and exits the introducer tip 2″′, as previously discussed.

It is recognized that the alternative configurations for any of the embodiments contemplated herein may be employed similarly, replacing for example, any one or more of the holding device assemblies 12, grip housing 3, connection end at the proximal end of the catheter 9, or the catheter, and one of skill in the art can utilize the teachings herein to select the embodiment that is suitable for the desired application.

### EXAMPLES

### Example 1

### Device to Anesthetize Urethra of a Living Being

The teachings herein may be employed to provide anesthetization to the urinary tract, extending from at least the urethral opening to the neck of the bladder. For example, the embodiment depicted in FIG. 51 may be used to deliver an anesthetic as the device is advanced through the urinary tract, and once the catheter is in position, the catheter may allow for introduction of fluids, or removal of materials, or serve as a protective sheath through which additional devices may be directed.

With reference to the embodiment of FIG. 51, a protective assembly 10′″, having a holding device assembly 12′″, having a housing 3′″, a retaining device 4′″, and an introducer tip 2″′ with a removable cap 1″′ is provided. The holding device 12″′ is configured with an internal receiving passageway through which a catheter may be directed. The interior of the grip housing between the retaining device 4″′ and the introducer tip 2″′ provides a lubricating jelly, which may be capable of anesthetizing nerves. Within the removable cap 1″′, there is provided an amount of lubricating jelly, which will coat the exterior surface of the introducer tip 2″′.

The graduated tubular style catheter/protective sheath assembly 5″′ is a catheter 9′″ and a connection at the proximal end. The connection may be any suitable connection, such as a luer lock, a luer slip connection, and may provide a one-way valve. The connection may be a single connection as shown in FIG. 51, though it is contemplated that an alternative connection, such as branched connector may be provided instead, for example, the Y-connector as shown in FIGS. 10-15, and having a first port with a connection for attaching a device, and a second port, which may be a funnel opening, through which fluids and devices may be directed. One or both of the branches may be optionally sealed, with a cap or a plug, as appropriate. The catheter 9″′ as shown features graduation marks and may optionally provide a marker 130 that can slide along the catheter body to mark the depth of penetration of the catheter.

With the cap 1″′ removed, the introducer tip may be inserted into the urethral opening of a living being. The distal tip of the catheter 9″′ is advanced through the grip housing, where it is coated in the lubricating gel, and exits the introducer tip, which by virtue of being a slit seal will wipe and retain most of the gel coating to be retained within the grip housing 3″′, while leaving a thin film of lubricating gel on the exterior surface of the catheter as it is advanced out the introducer tip, and further up the urethra.

The practitioner may depress the plunger of the syringe 6 in as needed, in in incremental amounts, as the catheter 9″′ is advanced through the urethra towards the bladder, to deliver the anesthetizing gel from the distal tip and/or openings 5C of the catheter 9′″. The anesthetizing gel will reduce discomfort as the catheter is advanced and may additionally serve as a lubricant to facilitate advancement of the catheter through the urinary tract. Such an embodiment would be capable of anesthetizing the entire length of the urethra from the glans penis to the neck of the bladder, and may further anesthetize within the bladder, if necessary, by continued delivery of anesthetizing medicament once the distal tip 5b of the catheter 9 in place within the bladder

The catheter may then be caused to pass the neck of the bladder as taught previously.

If necessary, the syringe may be replaced with a different syringe by releasing the luer connection and securing a different syringe thereon. The practitioner may then proceed with the procedure as necessary, such as by draining the bladder, or delivery of a medicament through the catheter.

### Example 2

### Protective Sheath Device for Passage of a Medical Device

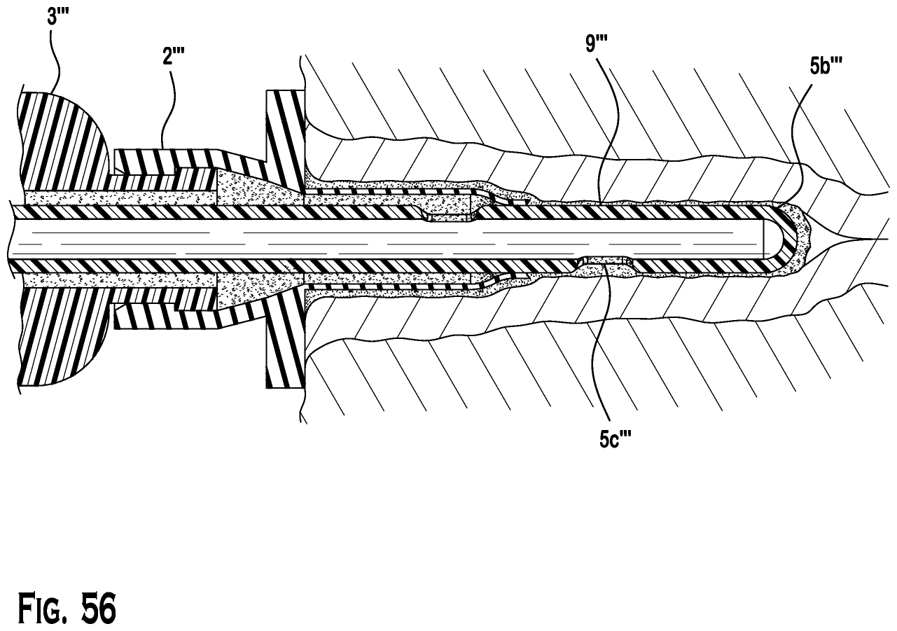

The various embodiments described herein may be deployed using the teachings herein into a tract of a living being, and once in place, the catheter may serve as a sheath for delivery of fluids, medicaments, devices through the interior of the catheter to treat the living being. For example, referring to the FIG. 35, a catheter having an open distal tip may be provided. The distal tip may optionally provide sidewall openings 5c as discussed previously. The proximal end of the catheter 9 may be provided with a Y-connector, as shown in FIG. 10. The catheter may be advanced through any of the holding device assemblies 12 taught herein, for example, the holding assembly depicted in FIG. 55, having a pair of large gripping surfaces that are slightly indented to prevent the practitioner's fingers from slipping off. The catheter may have a syringe secured to the second branch port 112 and containing an anesthetic to facilitate delivery of the catheter through the tract of a living being. The catheter may be positioned using the teachings of example 1. As shown in FIG. 56, the introducer tip 2′″ is directed into the tract of the living being, having been lubricated by the coating of lubricating jelly contained within the cap before being removed to expose tip 2″′. The introducer tip 2′″ is inserted until the broad shoulder of the introducer tip is urged against the tract opening, whereupon the catheter 9″′ may be advanced through the slit valve at the end of the introducer tip, and advanced into the tract, as will be familiar to those of skill in the art. In an embodiment where the grip housing 3″′ contains lubricating jelly within the body of the housing (as shown in FIGS. 7 and 55, the catheter exterior surface will be lubricated as it passes through the receiving passageway within the housing. If necessary, medicament, such as an anesthetic may be delivered to the tract via opening 5c″′.

Once in place, the second port of the Y connector may be unsealed, if necessary to drain the targeted region. The second port of the Y connector may be a funnel opening, through which a device may be inserted. The practitioner may note the positioning of the marker 130, if present, or alternatively, note the graduation mark where the catheter enters into the holding device assembly, and thereby know the distance the device is to be advanced through the catheter in order to reach or extend out from the open distal tip of the catheter 9. The device may then be advanced through the catheter 9 and exit out the open distal end of the catheter. An embodiment of the device 155, for example, as depicted in FIG. 60 may be any suitable device required to carry out a medical procedure, as determined by the practitioner. In an embodiment, the device 155 may be an endoscope, biopsy needle/punch, surgical fiber and/or culture swab, as appropriate for the procedure to be carried out.

### Example 3

### Device to Anesthetize Nasopharyngeal Region

The various embodiments described herein may be deployed, using the teachings herein, into a tract of a living being, and once in place, the catheter may serve as a sheath for delivery of fluids, medicaments, devices through the interior of the catheter to treat the living being. For example, the device of FIGS. 11 and 12 may be used to deliver an anesthetic as the device is advanced through nasal cavity towards the nasopharynx, and once the catheter is in position, the catheter may allow for introduction of fluids, or removal of materials, or serve as a protective sheath through which additional devices may be directed.

With reference to the embodiment of FIGS. 11 and 12, there is provided a protective assembly 10′, having a holding device assembly 12′, having a housing 3′, a retaining device 4′, and an introducer tip 2′ with a removable cap 1′ is provided. The holding device 12′ is configured with an internal receiving passageway through which a catheter 9′ may be directed. Within the removable cap 1′, there is provided an amount of lubricating jelly that will coat the exterior surface of the introducer tip 2′.

The graduated tubular style catheter/protective sheath assembly 5′ is a catheter 9′ and a connection at the proximal end. The connection may be any suitable connection, such as a luer lock, a luer slip connection, and may provide a one-way valve. The connection may be a branched connector, for example, the Y-connector as shown in FIGS. 11 and 12 and having a first port with a connection for attaching a device, such as a syringe or medicament delivery device, and a second port, which may be a funnel opening, through which fluids and devices may be directed. One or both of the branches may be optionally sealed, with a cap or a plug, as appropriate. Alternatively, the graduated tubular style catheter/protective sheath assembly 5′is provided with a single connection, for example as shown in FIG. 16 having a syringe connected to the proximal end of the catheter 9′ by a luer connection. The catheter 9′ as shown features graduation marks and may optionally provide a marker 130 that can slide along the catheter body to mark the depth of penetration of the catheter into the tract.

With the cap 1′ removed, the introducer tip may be inserted into the nostril opening of a living being. The retention device 4′ may be adjusted to be away from the tapered sides of the grip housing 3′, to allow free movement of the catheter through the grip housing 3′. The distal tip of the catheter 9′ is advanced through the grip housing 3′, and exits the introducer tip 2′, and may be advanced into the nasal cavity towards the nasopharynx. Once the catheter 9′ is in the desired position, the retention device 4′ may be adjusted to encircle the tapered sides of the grip housing, and prevent unintended movement of the catheter, relative to the grip housing 3′. It is contemplated that one or more of the grip housing 3′ or catheter 9′ may be secured by use of securing tape, as will be familiar to those of skill in the art.

The distal tip of the catheter 9′ may be provided a misting tip, or aerosolizing tip, as is known to those of skill in the art, such that as the medicament is delivered by the syringe, or other delivery device suitable to deliver the anesthetizing material, whereby a mist, for example an amount of atomized medicament solution, is delivered into the sinus cavity to broadcast the anesthetizing agent or other medicament over at least the entire region to be treated. In this manner, the entire nasal cavity may be anesthetized, rather than relying on alternative methods that are less effective in widespread delivery of anesthetic. The anesthetizing medicament will reduce discomfort as the catheter 9′ is advanced.

If necessary, the syringe or medicament delivery device may be replaced with a device containing additional or different medication by releasing the luer connection and securing a different device thereon. The practitioner may then proceed with the procedure as necessary, such as by delivering a device through the catheter 9′, such as a culture swab, biopsy needle, surgical fiber, or delivery of a medicament through the catheter 9′.

### Example 4

### Device to Deliver UV light via Surgical Fiber Directed Through Protective Sheath

The various embodiments described herein may be deployed using the teachings herein into a tract of a living being, and once in place, the catheter may serve as a sheath for delivery of fluids, medicaments, devices through the interior of the catheter to treat the living being. For example, the device placed into the tract of a living being, according to example 2, may then serve as a protective sheath through which a device 155 that is a surgical fiber may be directed. In this manner, the surgical fiber and/or other medical devices 155 may be deployed as required into the living being, without causing discomfort to the patient, and avoids the possibility of repeated interventions resulting in disturbances to the epithelial layer. Further, by introducing the device through the sheath, the potential for infections is minimized, as the epithelial layer can remain intact by not being subjected to repeated disruptions, and the introduction of bacterial contamination can be avoided.

In the instance that a surgical fiber is to be deployed through the catheter embodiment, the surgical fiber device 155 may directed through an open port at the proximal end of the catheter. Where the proximal end of the catheter is the single luer connection as depicted in FIG. 1, the surgical fiber may be sized to fit through the connector, and or may alternatively be directed through a fitting or valve secured to the luer connector. In an embodiment where the proximal end of the connector has a Y-connector 100, as depicted in FIG. 13, the surgical fiber may be directed through the funnel opening 120, or the luer connector port 112.

The device 155 that is in the form of a surgical fiber may be a fine light transmitting fiber, such as an optical fiber, as is known to be used for laser surgery, and sized to pass through the interior of the catheter 9. The surgical fiber may be connected to a UV light source 150, such as a UV generating bulb generating light emissions in a wavelength band that is known to have germicidal properties for example, UV-B and/or UV-C. In an embodiment, the UV light source produces significant light output at 254 nm. In an embodiment, the proximal end of the surgical fiber may be joined to a portable handheld device that has the UV generating bulb.

Once the surgical fiber is in place, as taught by Example 2, the light may be activated and applied to the treatment region for a duration of time sufficient to have a germicidal effect, and in this embodiment may be usefully employed where there is an internal infection, such as a urinary tract infection, to reduce or eliminate the source of infection. In another embodiment, the UV light delivered by surgical fiber may be directed through the protective sheath into a tract, for example the respiratory pathway, such as a nasal cavity or a lung, and thereby treat an infection with the germicidal UV light.

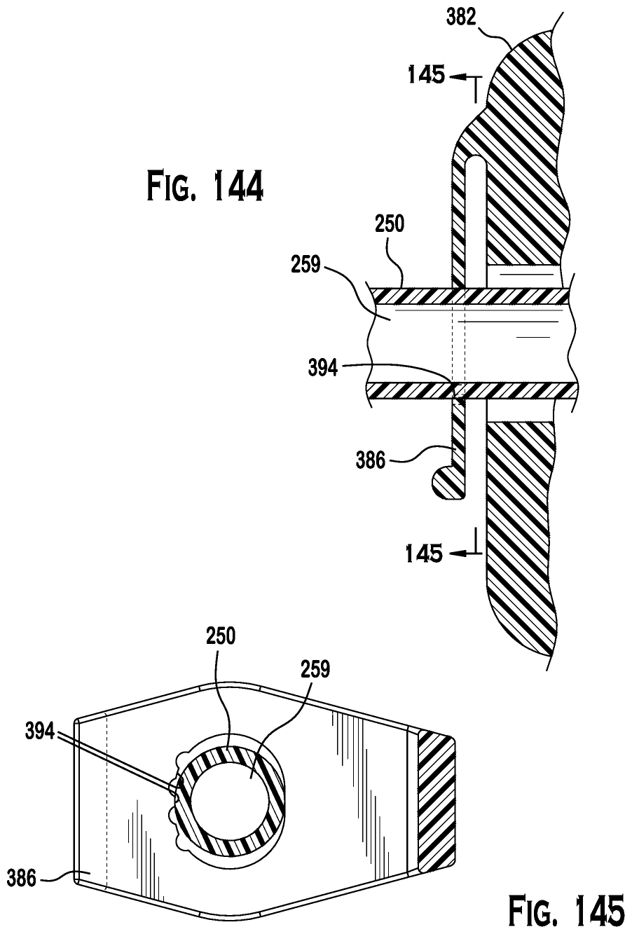

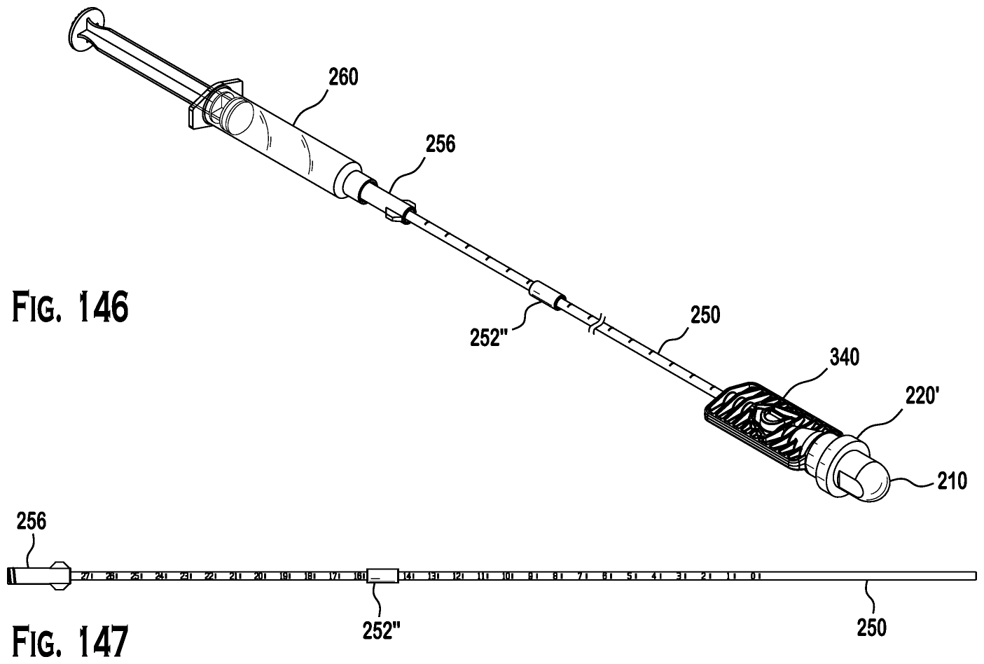

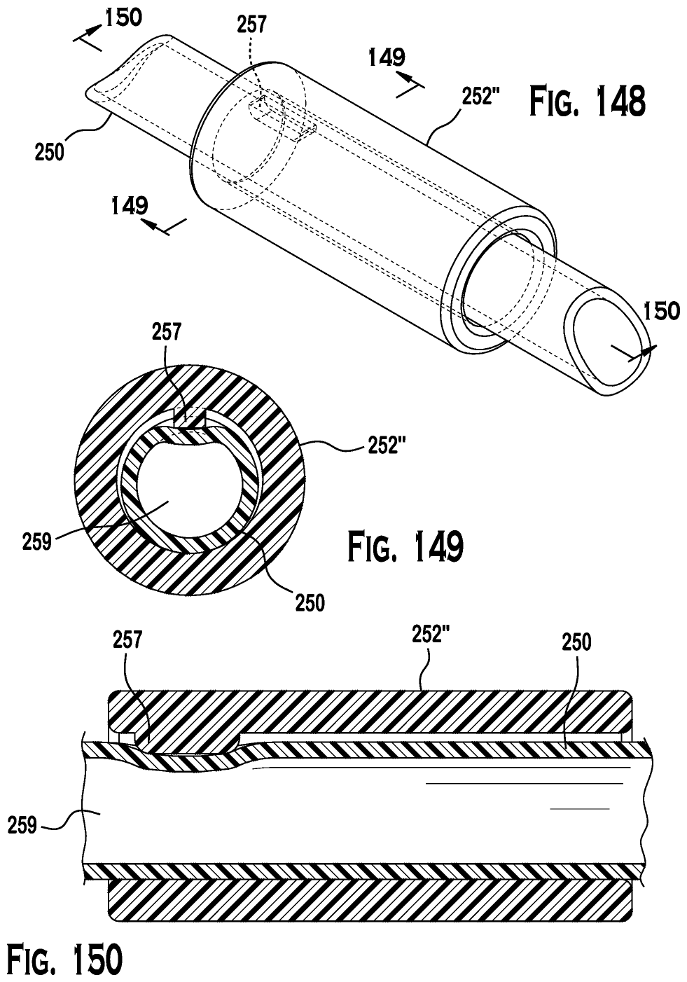

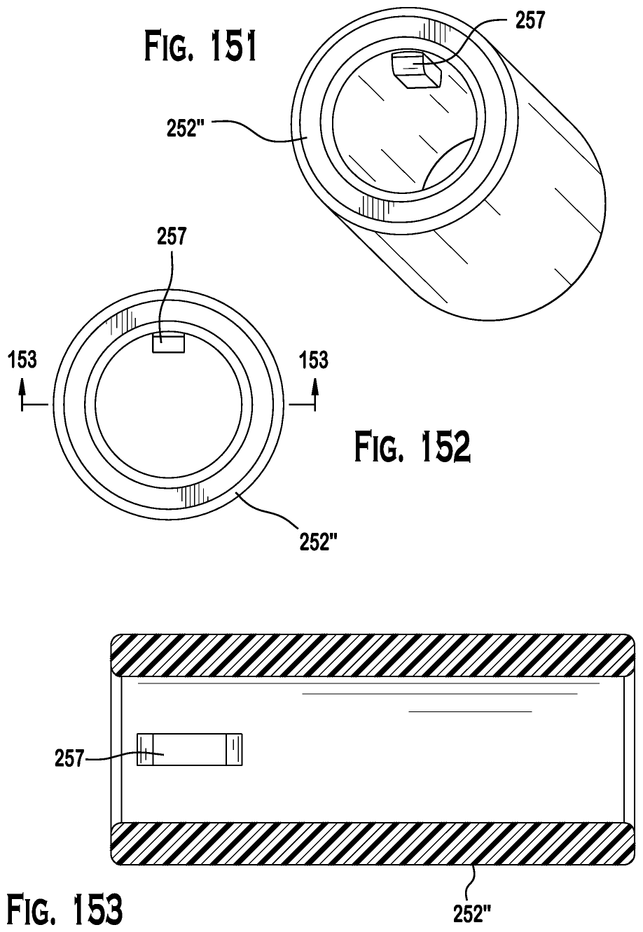

Referring to FIGS. 61-153, a catheter assembly 200, is generally constructed in accordance with the following major components: a cap 210, an introducer tip 220, a variation of a grip housing 320, 340, 360, 380, a tube gripper 270, and a catheter 250 as shown in FIG. 61 and FIG. 145.

In the exemplary embodiment, a cap 210 is a covering member, much like a known cap for a catheter tip. One of ordinary skill in the art would understand the applicant's design is not the exclusive embodiment. The cap 210 is plastic, such as polyvinyl chloride (PVC), but can be made from other known plastics. One of ordinary skill in the art would understand the applicant's design is not the exclusive embodiment. The cap 210 should be easily removed but should not pull an introducer tip 220 off of a grip housing when removed.

### First Variation Of Introducer Tip

In the exemplary embodiment, there are two variations of introducer tip 220′, and 220″. The introducer tip 220′ generally includes the following major components: an elongated cylindrical member 222′, a protruding end piece 224′, and a biasing surface 228′.

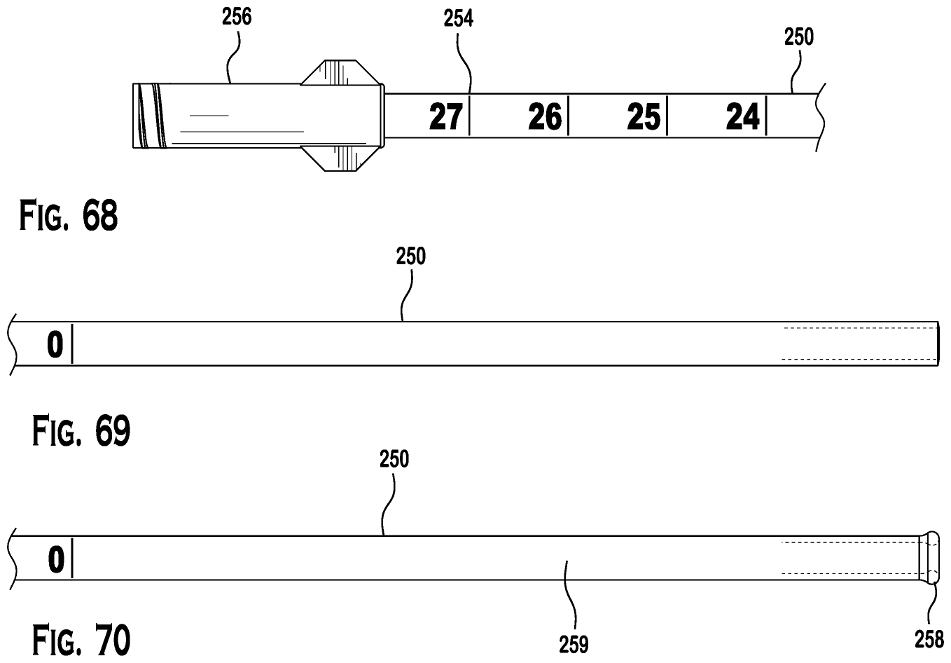

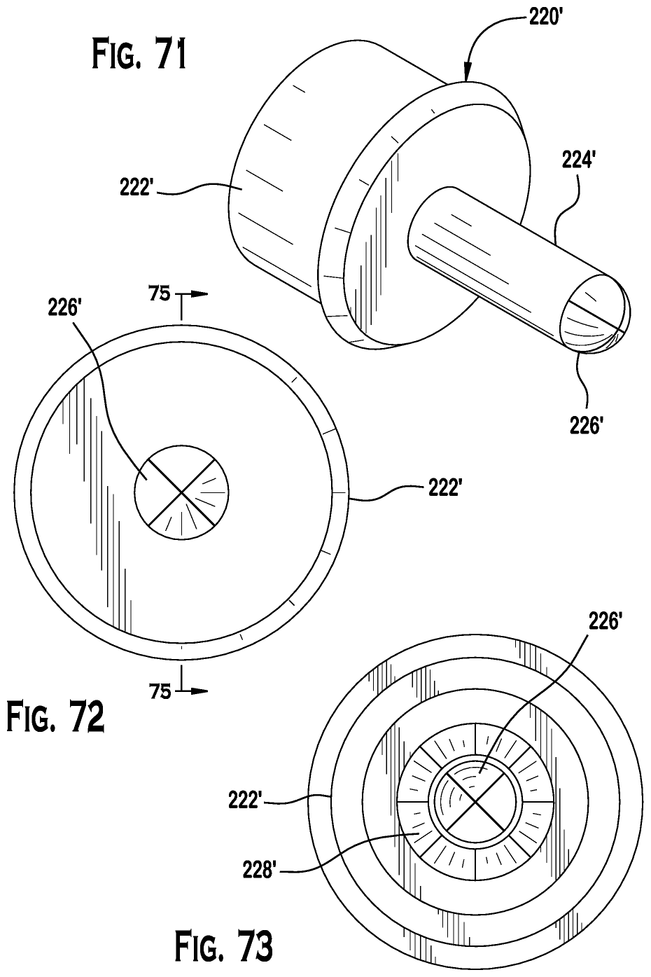

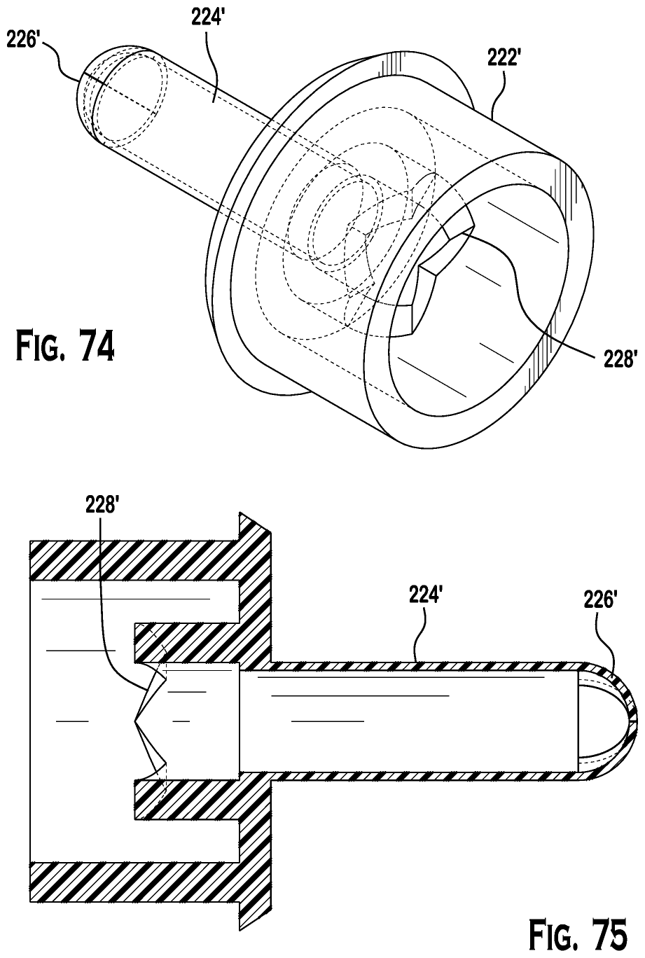

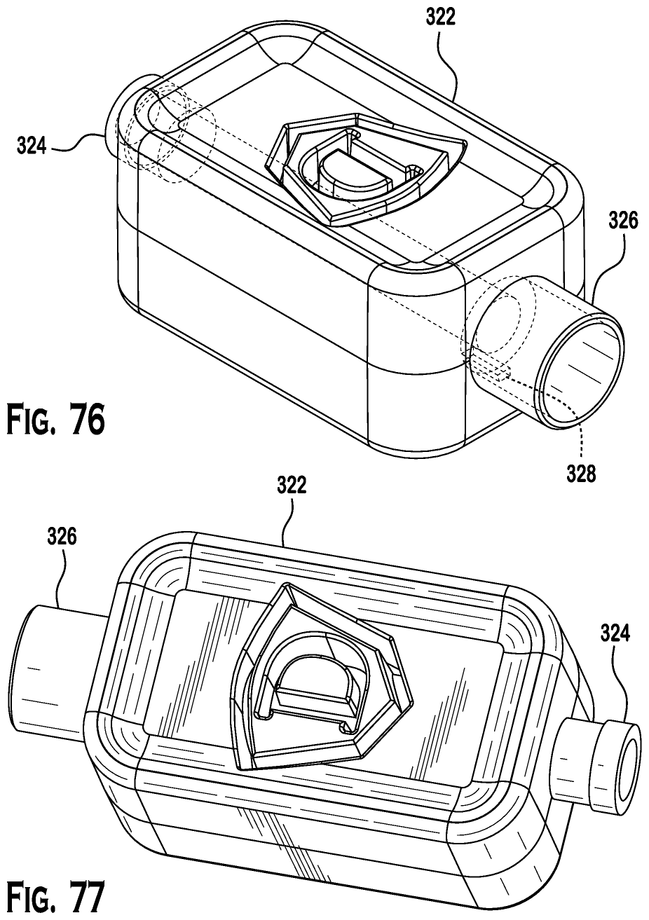

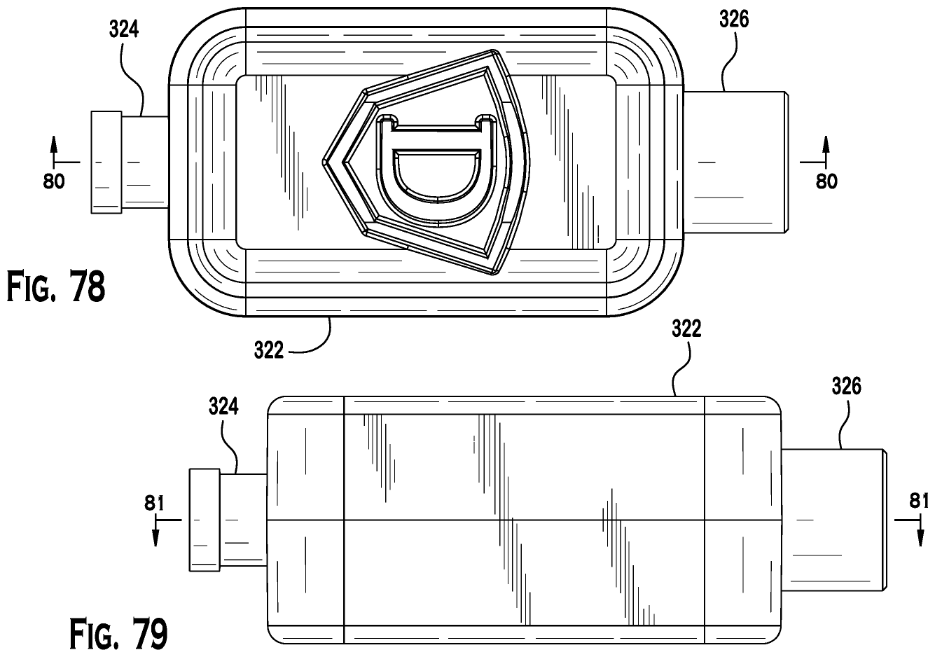

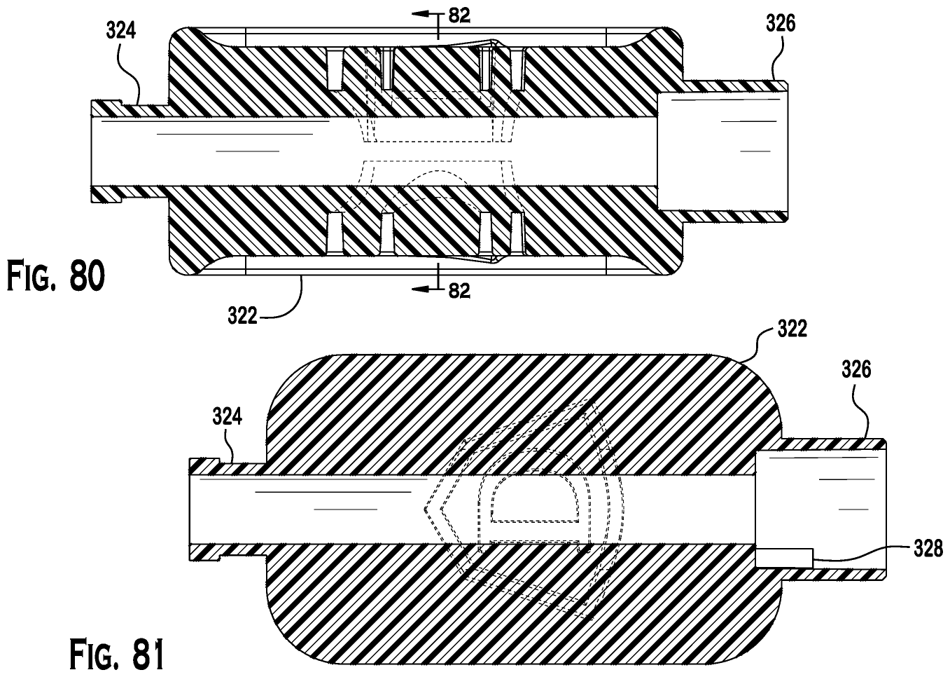

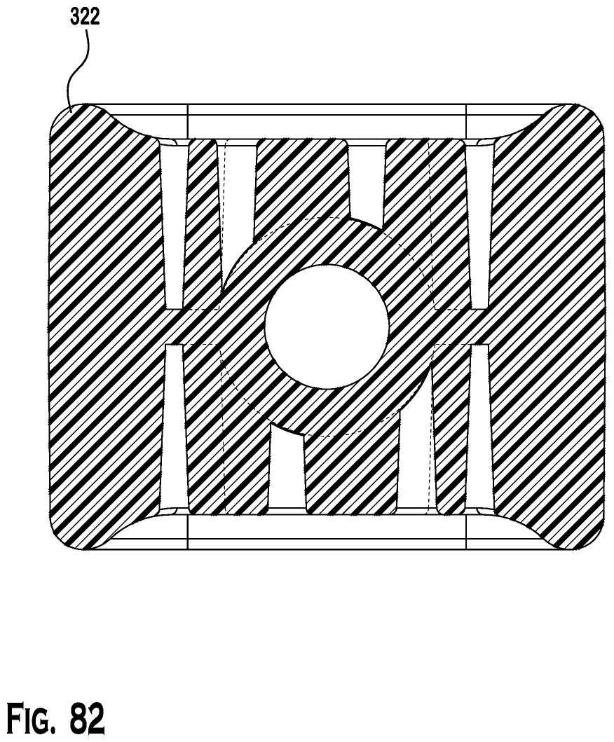

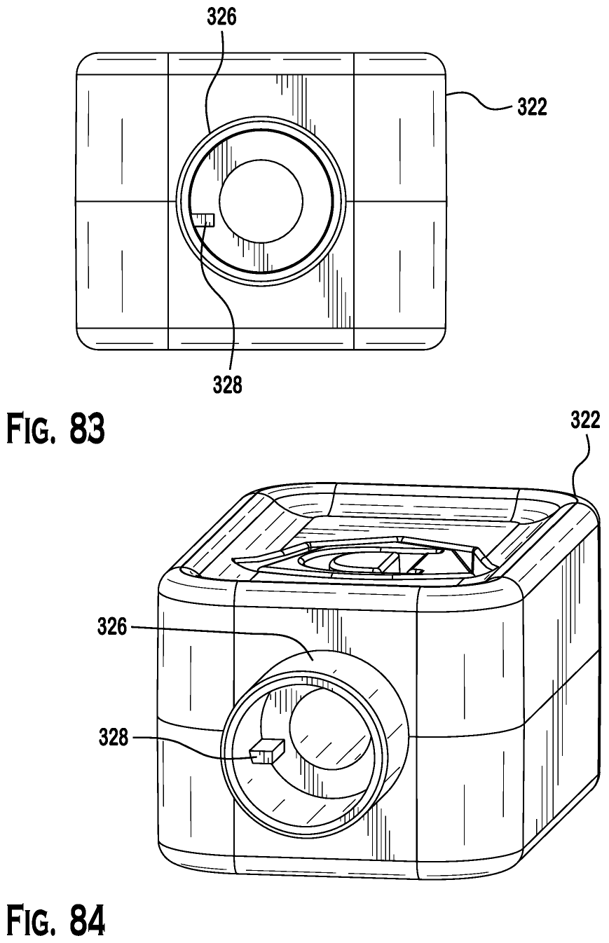

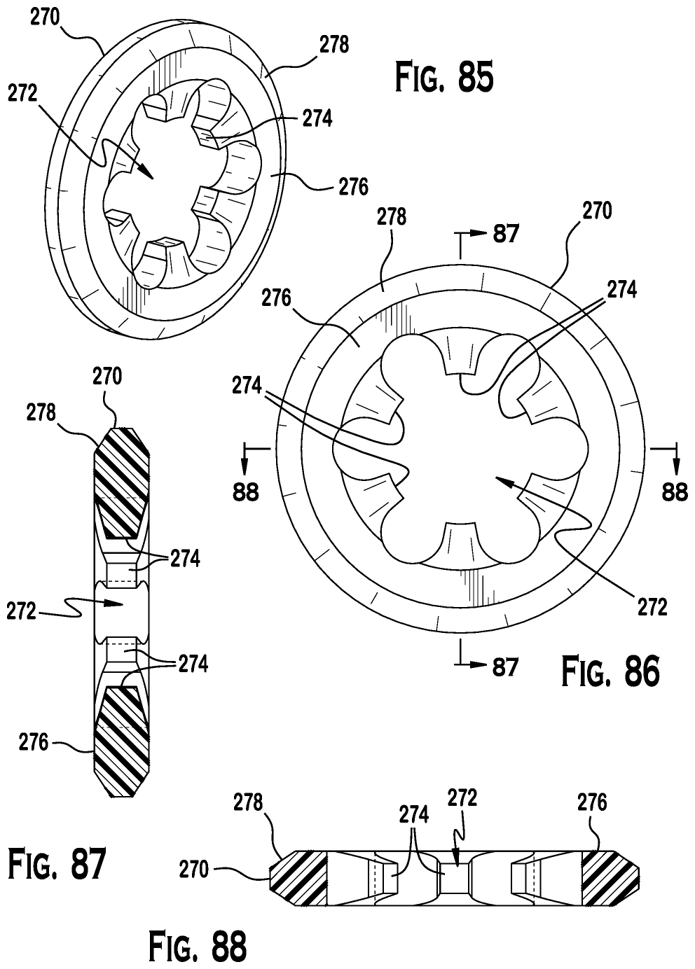

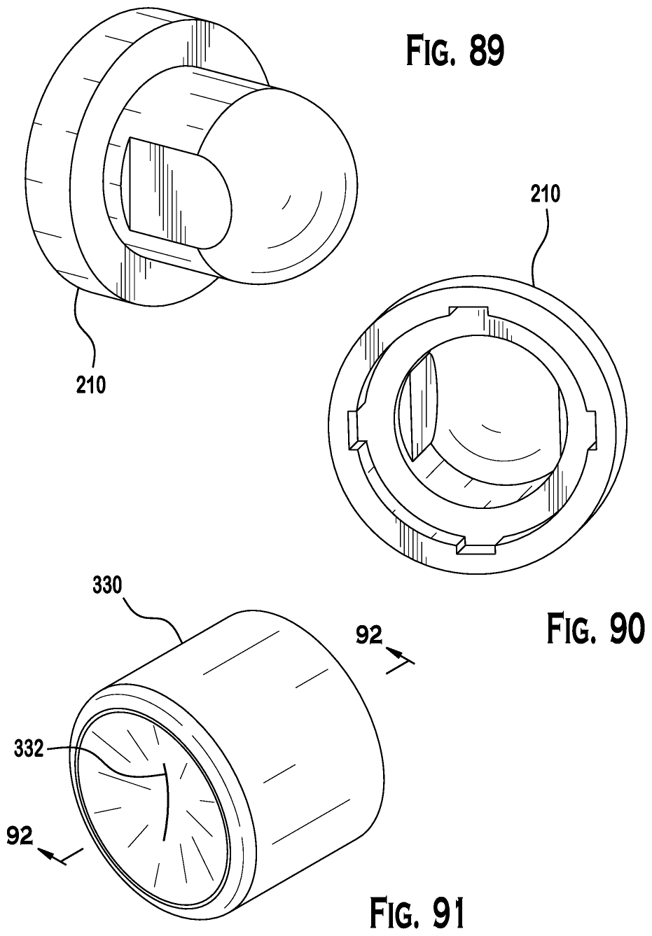

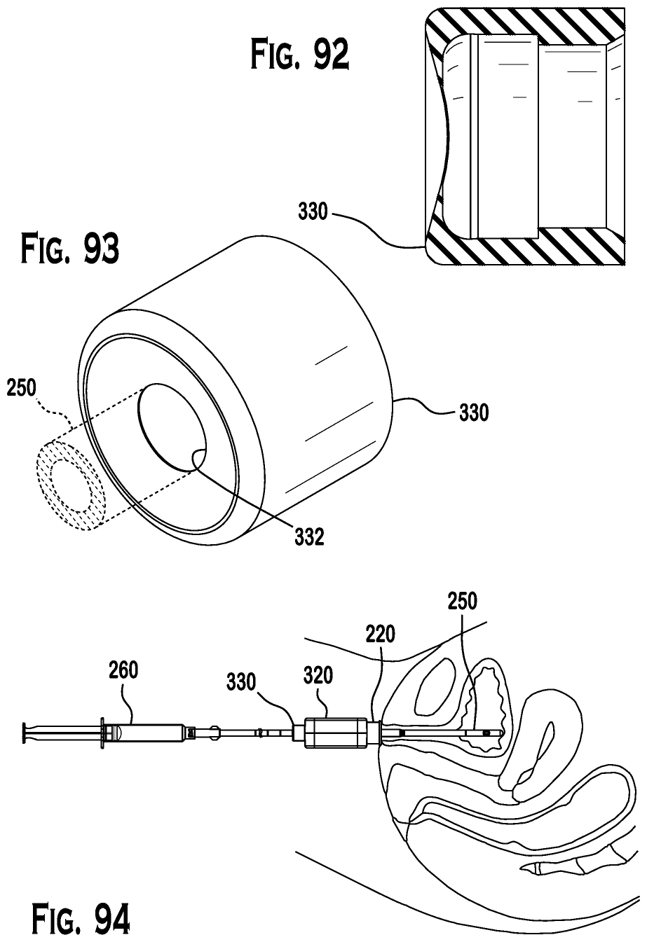

In the exemplary embodiment the elongated cylindrical member 222′ is made from soft silicone, though it is contemplated that other soft, flexible materials may be utilized instead, such as rubber or other flexible polymers. One of ordinary skill in the art would understand the applicant's design is not the exclusive embodiment. The elongated cylindrical member 222′ includes a tip connector catheter receiving passageway 221′ extending there through. One of ordinary skill in the art would understand applicant's design is not the exclusive embodiment. In the exemplary embodiment, the protruding end piece 224′ is cylindrical and located in the central region of the elongated cylindrical member 222′. One of ordinary skill in the art would understand the applicant's design is not the exclusive embodiment. The protruding end piece 224′ further includes a distal tip end 226′. The distal tip end 226′ includes a slit seal, which may be an “X” shaped cut, as shown in FIG. 71. One of ordinary skill in the art would understand the applicant's design is not the exclusive embodiment. In the exemplary embodiment, the biasing surface 228′ is a protruding wall. The biasing surface 228′ is located on the inside of the elongated cylindrical member 222′. One of ordinary skill in the art would understand the applicant's design is not the exclusive embodiment. The biasing surface 228′ further includes protruding teeth 230′. One of ordinary skill in the art would understand the applicant's design is not the exclusive embodiment. The protruding teeth 230′ of the biasing surface 228′ allows the biasing surface 228′ to grip a tube gripper 270.

### Second Variation of Introducer Tip

In the exemplary embodiment, the second variation of introducer tip 220″, generally includes the following major components: an elongated cylindrical member 222″, and a protruding end piece 224″.

In the exemplary embodiment the elongated cylindrical member 222″ is made from soft silicone, though it is contemplated that other soft, flexible materials may be utilized instead, such as rubber or other flexible polymers. One of ordinary skill in the art would understand the applicant's design is not the exclusive embodiment. The elongated cylindrical member 222″ includes a tip connector catheter receiving passageway 221″ extending there through. One of ordinary skill in the art would understand the applicant's design is not the exclusive embodiment. In the exemplary embodiment, the protruding end piece 224″ is cylindrical and located in the central region of the elongated cylindrical member 222″. One of ordinary skill in the art would understand the applicant's design is not the exclusive embodiment. The protruding end piece 224″ further includes a distal tip end 226″. One of ordinary skill in the art would understand the applicant's design is not the exclusive embodiment. The distal tip end 226″ includes a slit seal, which may be an “X” shaped cut, as shown in FIG. 71. One of ordinary skill in the art would understand the applicant's design is not the exclusive embodiment.

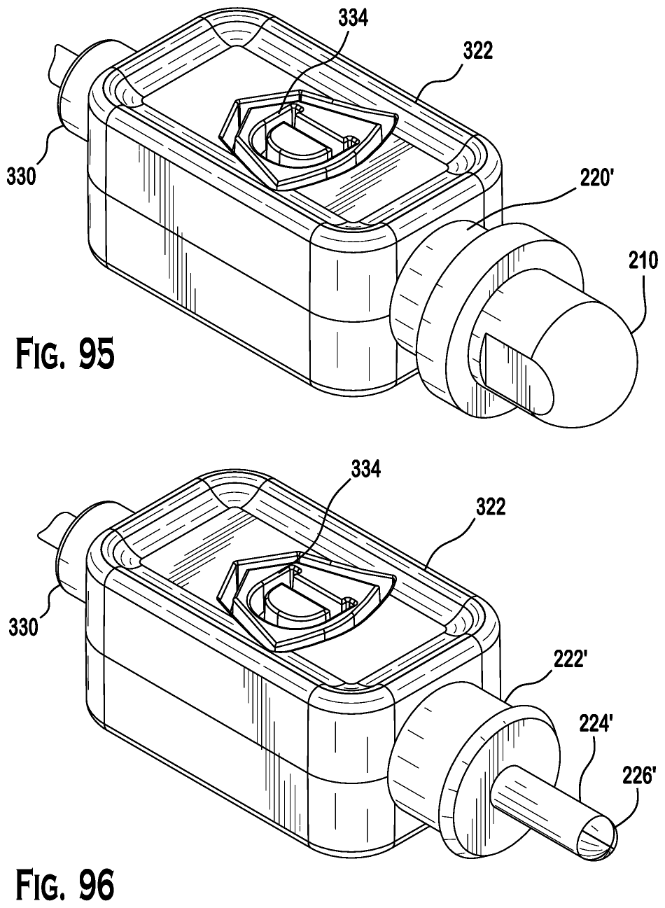

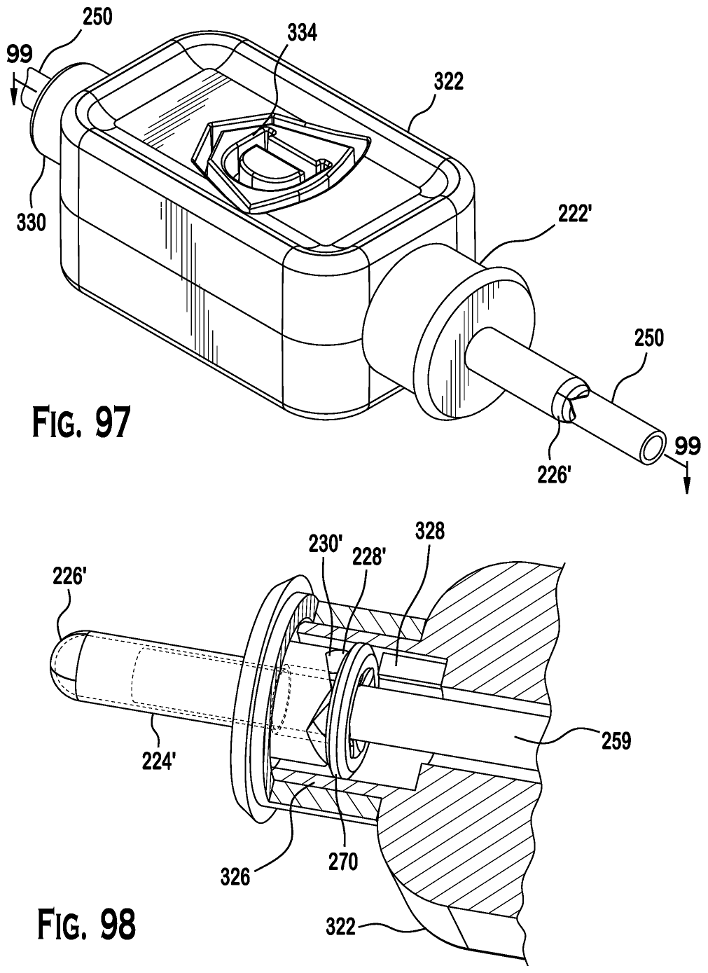

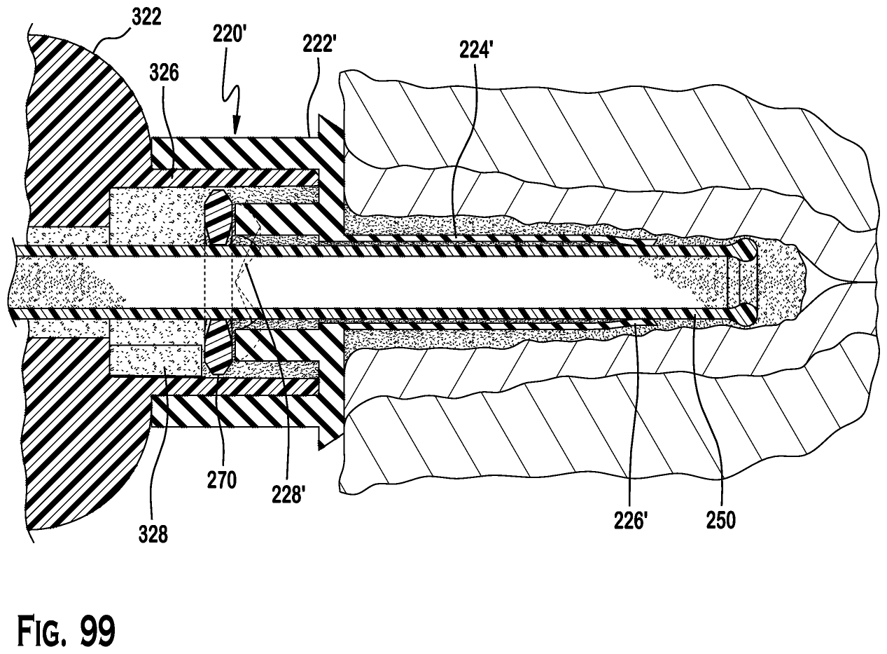

In the exemplary embodiment, as shown in FIG. 95, FIG. 104, FIG. 126, and FIG. 139, there are four total variations of a grip housing 320, 340, 360, 380 each with distinctive features. One of ordinary skill in the art would understand the applicant's design of the housing is not the exclusive embodiment and other variations can be used.

### First Variation of Housing

In the exemplary embodiment, the grip housing 320 generally includes a housing 322. In the exemplary embodiment, the housing 322 is a rectangular hollow structure. One of ordinary skill in the art would understand the applicant's design is not the exclusive embodiment. The housing 322 includes a housing receiving passageway 324 at one end thereof. The housing receiving passageway 324 is a circular member with a through hole extending the length of the housing 322. One of ordinary skill in the art would understand the applicant's design is not the exclusive embodiment. The housing 322 further includes an introducer tip connector 326 located at the opposite end from the housing receiving passageway 324. One of ordinary skill in the art would understand the applicant's design is not the exclusive embodiment. The housing 322 further includes a leveling surface 328. The leveling surface 328 is a protrusion on the inside of the introducer tip connector 326. One of ordinary skill in the art would understand the applicant's design is not the exclusive embodiment. The housing 322 further includes a catheter receiver 330. The catheter receiver 330 further includes a catheter slit 332. One of ordinary skill in the art would understand the applicant's design is not the exclusive embodiment. The housing 322 further includes a grip 334. The grip 334 is an indented D-shape. One of ordinary skill in the art would understand the applicant's design is not the exclusive embodiment.

### Second Variation of Housing

In the exemplary embodiment, the grip housing 340 generally includes a housing 342. In the exemplary embodiment, the housing 342 is a rectangular hollow structure. One of ordinary skill in the art would understand the applicant's design is not the exclusive embodiment. The housing 342 includes a housing receiving passageway 344 at one end thereof. The housing receiving passageway 344 is a circular member with a through hole extending the length of the housing 342. One of ordinary skill in the art would understand the applicant's design is not the exclusive embodiment. The housing 342 further includes an introducer tip connector 346 located at the opposite end from the housing receiving passageway 344. One of ordinary skill in the art would understand the applicant's design is not the exclusive embodiment. The introducer tip connector 346 further includes an elevated set of rings 348 located on the outside of the introducer tip connector 346. The housing 342 further includes a leveling surface 350. The leveling surface 350 are four protrusions on the inside of the introducer tip connector 346. One of ordinary skill in the art would understand the applicant's design is not the exclusive embodiment. The housing 342 further includes a grip 352. The grip 352 is raised webbing with an indented D shape with a shield around it and located in the outer region of the housing 342. One of ordinary skill in the art would understand the applicant's design is not the exclusive embodiment.

### Third Variation of Housing

In the exemplary embodiment, the grip housing 360 generally includes a housing 362. In the exemplary embodiment, the housing 362 is a rectangular structure. One of ordinary skill in the art would understand the applicant's design is not the exclusive embodiment. The housing 362 includes a protruding housing receiving passageway 364 at one end thereof. The protruding housing receiving passageway 364 is an elongated circular member with a through hole extending the length of the housing 362. One of ordinary skill in the art would understand the applicant's design is not the exclusive embodiment. The housing receiving passageway 364 further includes mirroring clasps 366 located on opposite sides of the central region thereof. One of ordinary skill in the art would understand the applicant's design is not the exclusive embodiment. The housing 362 further includes an introducer tip connector 368. The introducer tip connector 368 is circular. One of ordinary skill in the art would understand the applicant's design is not the exclusive embodiment. The introducer tip connector 368 is located at the opposite end from the protruding housing receiving passageway 364. The introducer tip connector 368 further includes an elevated set of rings 370 located on the outside of the introducer tip connector 368. The housing 362 further includes a grip 372. The grip 372 is raised webbing with an indented D shape with a shield around it and located in the outer region of the housing 362. One of ordinary skill in the art would understand the applicant's design is not the exclusive embodiment.

### Fourth Variation of Housing

In the exemplary embodiment, the grip housing 380 generally includes a housing 382. In the exemplary embodiment, the housing 382 is a rectangular structure. One of ordinary skill in the art would understand the applicant's design is not the exclusive embodiment. The housing 382 includes a housing receiving passageway 384 at one end thereof. The housing receiving passageway 384 is a circular member with a through hole extending the length of the housing 382. One of ordinary skill in the art would understand the applicant's design is not the exclusive embodiment. The housing 382 further includes a catheter securing flap 386. The catheter securing flap 386 is a plate like structure with a through hole in the central region and is fixed at one end. One of ordinary skill in the art would understand the applicant's design is not the exclusive embodiment. The catheter securing flap 386 further includes a grooved catheter grip 394. The grooved catheter grip 394 are indentations. One of ordinary skill in the art would understand the applicant's design is not the exclusive embodiment. The housing 382 further includes an introducer tip connector 388. The introducer tip connector 388 is circular. One of ordinary skill in the art would understand the applicant's design is not the exclusive embodiment. The introducer tip connector 388 is located at the opposite end from the housing receiving passageway 384. The introducer tip connector 388 further includes an elevated set of rings 390 around the outside of the introducer tip connector 388. One of ordinary skill in the art would understand the applicant's design is not the exclusive embodiment. The housing 382 further includes a grip 392. The grip 392 is raised webbing with an indented D shape with a shield around it and located in the outer region of the housing 382. One of ordinary skill in the art would understand the applicant's design is not the exclusive embodiment.

In the exemplary embodiment, the tube gripper 270 is a ring. One of ordinary skill in the art would understand the applicant's design is not the exclusive embodiment. The tube gripper 270 generally includes a chamfered edge 278 on the outer edge of the tube gripper 270. One of ordinary skill in the art would understand the applicant's design is not the exclusive embodiment. The tube gripper 270 further includes a top face 276 which is adjacent to the chamfered edge 278. One of ordinary skill in the art would understand the applicant's design is not the exclusive embodiment. The tube gripper 270 further includes a bottom face 277 which is located opposite of the top face 276. The tube gripper 270 further includes a gripping surface 274. The gripping surface 274 are teeth like protrusions from the top face 276. One of ordinary skill in the art would understand the applicant's design is not the exclusive embodiment. The tube gripper 270 further includes a catheter orifice 272 running through the center of the tube gripper 270. One of ordinary skill in the art would understand the applicant's design is not the exclusive embodiment. As a whole, the tube gripper 270 can be formed in different shapes and sizes and applicant's design of the tube gripper 270 is not the exclusive embodiment.

In the exemplary embodiment, the catheter 250 is a tube. One of ordinary skill in the art would understand the applicant's design is not the exclusive embodiment. The catheter 250 includes a female luer lock connector 256 located at one end of the catheter 250. One of ordinary skill in the art would understand the applicant's design is not the exclusive embodiment. The catheter 250 further includes a bulbus head 258 at the opposite end from the female luer lock connector 256. One of ordinary skill in the art would understand the applicant's design is not the exclusive embodiment. The catheter 250 further includes graduated marks 254, which is graduated in centimeters and will allow the practitioner to note the actual depth that the catheter 250 has entered the body and the distance the medication was dispensed. One of ordinary skill in the art would understand the applicant's design is not the exclusive embodiment. The catheter 250 further includes a donut shaped marker 252′. One of ordinary skill in the art would understand the applicant's design is not the exclusive embodiment. In one embodiment, the catheter 250 further includes an elongated donut shaped marker 252″. The elongated donut shaped marker 252″ further includes a rectangular protrusion 257. One of ordinary skill in the art would understand the applicant's design is not the exclusive embodiment. The catheter 250 further includes a catheter passage 259. One of ordinary skill in the art would understand the applicant's design is not the exclusive embodiment.

In the exemplary embodiment, the catheter assembly 200, is generally constructed in accordance with the following major components: a cap 210, an introducer tip 220′, 220″, a grip housing 320, 340, 360, 380 a tube gripper 270, and a catheter 250 as shown in FIG. 61-153.

As a general matter, when using the grip housing 320, the tube gripper 270 is inserted into the inner edge of the introducer tip connector 326. The introducer tip 220′ is fastened to the introducer tip connector 326 of the housing 322 by the elongated cylindrical member 222′. The catheter 250 is passed through the catheter receiver's 330 catheter slit 332 and into the housing receiving passage 324 of the housing 322. The catheter 250 is inserted through the catheter orifice 272 of the tube gripper 270. The catheter 250 is then passed through and into the tip connector catheter receiving passageway 221′ of the introducer tip 220′. The catheter 250 is then passed out of the distal end tip 226′ of the protruding end piece 224′. The cap 210 is fitted to the introducer tip 220′ until the catheter assembly 200 is ready to be used.

As a general matter, when using the grip housing 340, the tube gripper 270 is inserted into the inner edge of the introducer tip connector 346. The introducer tip 220′ is fastened to the introducer tip connector 346 of the housing 342 by the elongated cylindrical member 222′. The catheter 250 is passed through and into the housing receiving passage 344 of the housing 342. The catheter 250 is inserted through the catheter orifice 272 of the tube gripper 270. The catheter 250 is then passed through and into the tip connector catheter receiving passageway 221′ of the introducer tip 220′. The catheter 250 is then passed out of the distal end tip 226′ of the protruding end piece 224′. The cap 210 is fitted to the introducer tip 220′ until the catheter assembly 200 is ready to be used.

As a general matter, when using the grip housing 360, the introducer tip 220″ is fastened to the introducer tip connector 368 of the housing 362 by the elongated cylindrical member 222″. The catheter 250 is passed through and into the housing receiving passage 364 of the housing 362. The catheter 250 is then passed through and into the tip connector catheter receiving passageway 221″ of the introducer tip 220″. The catheter 250 is then passed out of the distal end tip 226″ of the protruding end piece 224″. The cap 210 is fitted to the introducer tip 220″ until the catheter assembly 200 is ready to be used.

As a general matter, when using the grip housing 380, the introducer tip 220″ is fastened to the introducer tip connector 388 of the housing 382 by the elongated cylindrical member 222″. The catheter 250 is passed through the catheter securing flap 386 and into the housing receiving passage 384 of the housing 382. The catheter 250 is then passed through and into the tip connector receiving passageway 221″ of the introducer tip 220″. The catheter 250 is then passed out of the distal end tip 226″ of the protruding end piece 224″. The cap 210 is fitted to the introducer tip 220″ until the catheter assembly 200 is ready to be used.

In the exemplary embodiment, the catheter assembly 200 as a whole works in conjunction with a female luer lock connector 256, and a syringe 260 to slowly dispense a medication and/or an anesthetic gel such as Lidocaine. Once the urethra is fully anesthetized, the catheter assembly 200 would be connected to a sealed 500 mL rectangular bag (not shown) by the luer lock connector 256 to allow for drainage of the urine. Moreover, once drainage has been complete, a cap (not shown) is attached to the sealed 500 mL rectangular bag to allow for sealed containment of the urine.

In the exemplary embodiment, the catheter assembly 200 is opened from the sterile packaging and securely attached to the syringe 260 by the female luer lock connector 256. The penis is held upright, from a ventral position, the introducer tip 220′,220″ is inserted into the glans penis, and moved forward into the entrance of the urethra. The introducer tip 220′,220″ is designed to protect the catheter 250 from being contaminated yielding a safe trek past the region of the urethra commonly known to be a repository for a wide variety of pathogenic bacteria. The catheter 250 is slowly advanced while irrigating the entire length of the urethra and dispensing the medication and/or the lidocaine gel from the syringe 260 all the way to the sphincter. Once the desired depth is reached the practitioner can mark off the depth in centimeters on the catheter 250 using the donut shaped marker 252′ or the elongated donut shaped marker 252″. The practitioner can lock the depth of the marker in place by using the rectangular protrusion 257 which provides a firm grip of the catheter 250. Further, this allows the practitioner to record the depth that the catheter 250 tracked and the distance the medication was dispensed. The catheter assembly 200 provides the patient a reduced or pain free examination and gives the urologist an unrestricted free access into the bladder. The catheter assembly 200 is able to irrigate the entire urethra with an anesthetic from the glans penis to the bladder sphincter.

When using either the grip housing 320 or the grip housing 340 with the catheter assembly 200, the tube gripper 270 of the catheter assembly 200 is configured to move about and between a first position and a second position. In the first position, a portion of the top face 276 of the tube gripper 270 is in contact with the leveling surface 328,350, such that the catheter orifice 272 of the tube gripper 270 is positioned substantially co-axial to the catheter passage 259. In the second position, at least a portion of the bottom face 277 of the tube gripper 270 is in contact with the biasing surface 228′ of the introducer tip 220′ such that the catheter orifice 272 is positioned at an acute angle with respect to the axis of the housing receiving passageway 324,344. In one aspect, the tube gripper 270 is configured such that, upon application of an external force on the catheter 250 resulting in longitudinal movement of the catheter 250 in the first direction, the tube gripper 270 is positioned in the first position adjacent the leveling surface 328,350 of the grip housing 320,340. The biasing surface 228′ is able to grip the tube gripper 270 by the protruding teeth 230′. Moreover, the tube gripper 270 is configured such that, upon the application of an external force on the catheter 250 resulting in longitudinal movement of the catheter 250 in the second direction, the tube gripper 270 is positioned in the second position adjacent the biasing surface 228′.

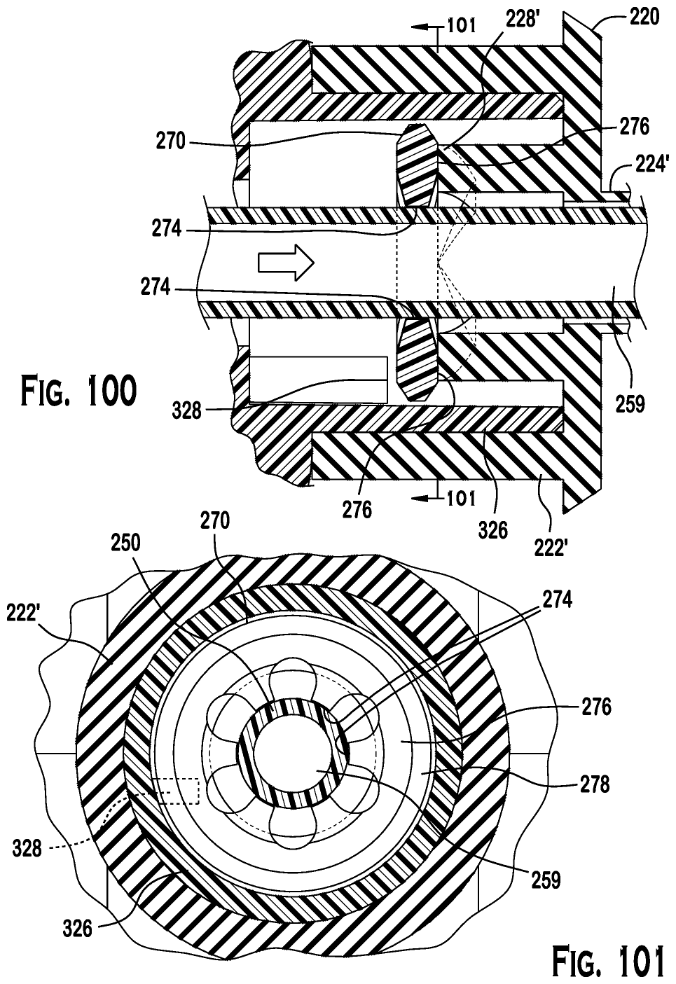

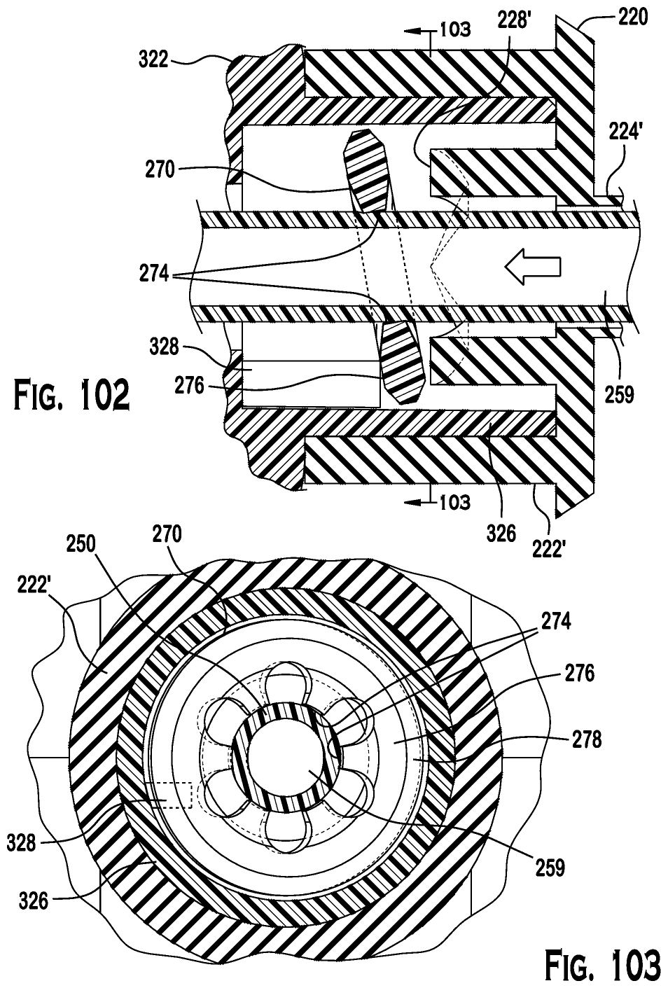

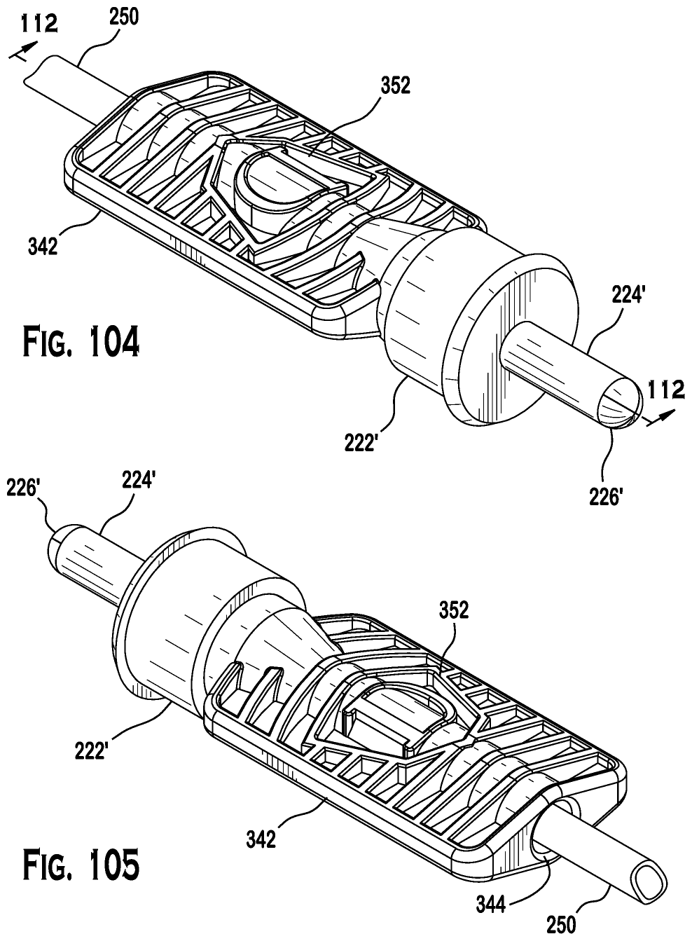

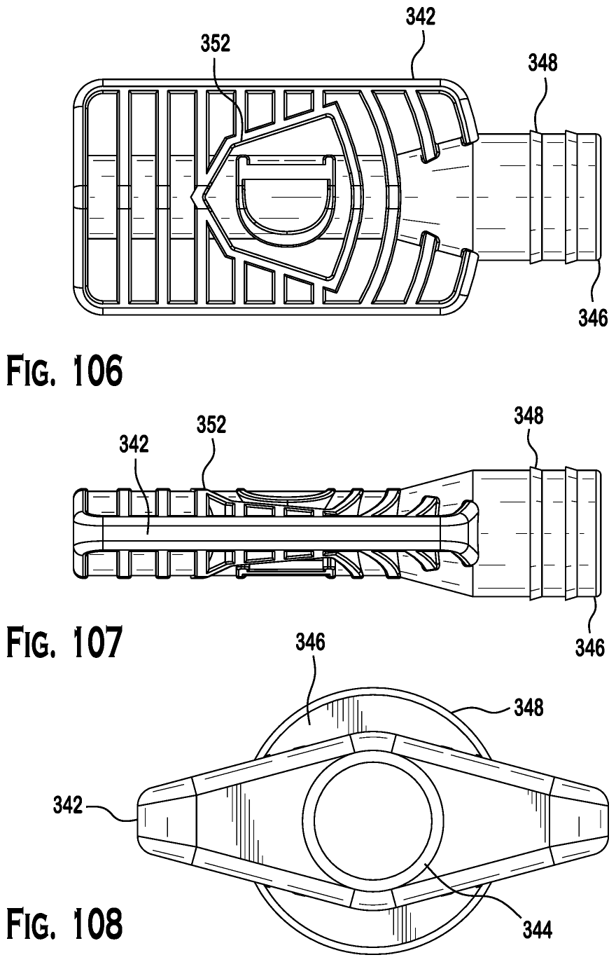

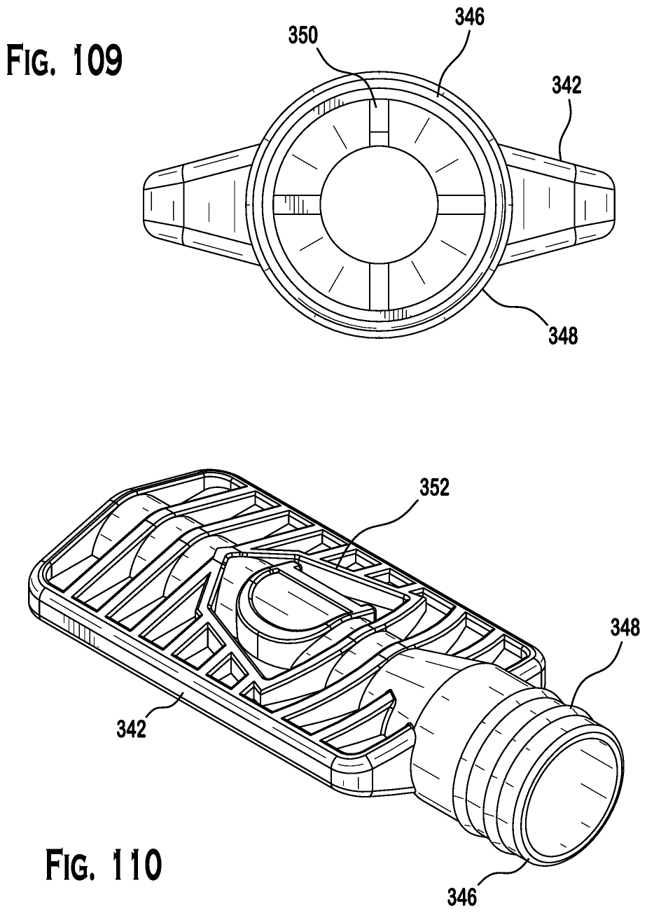

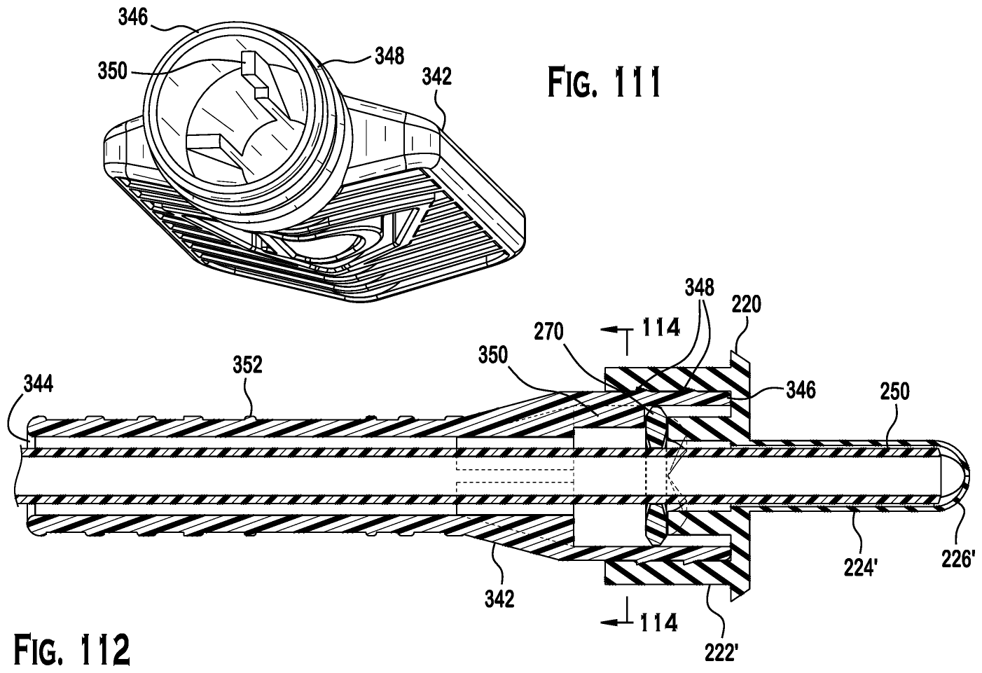

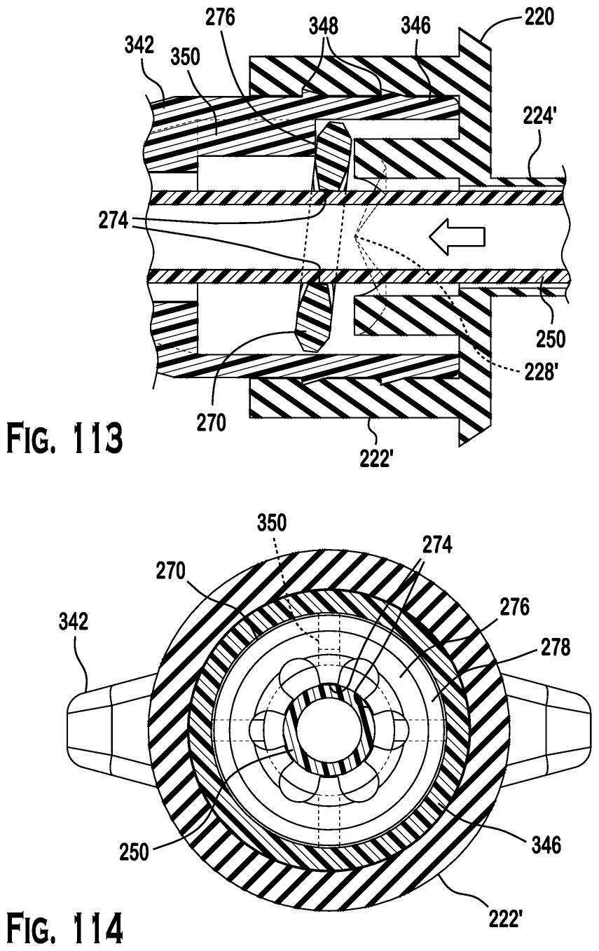

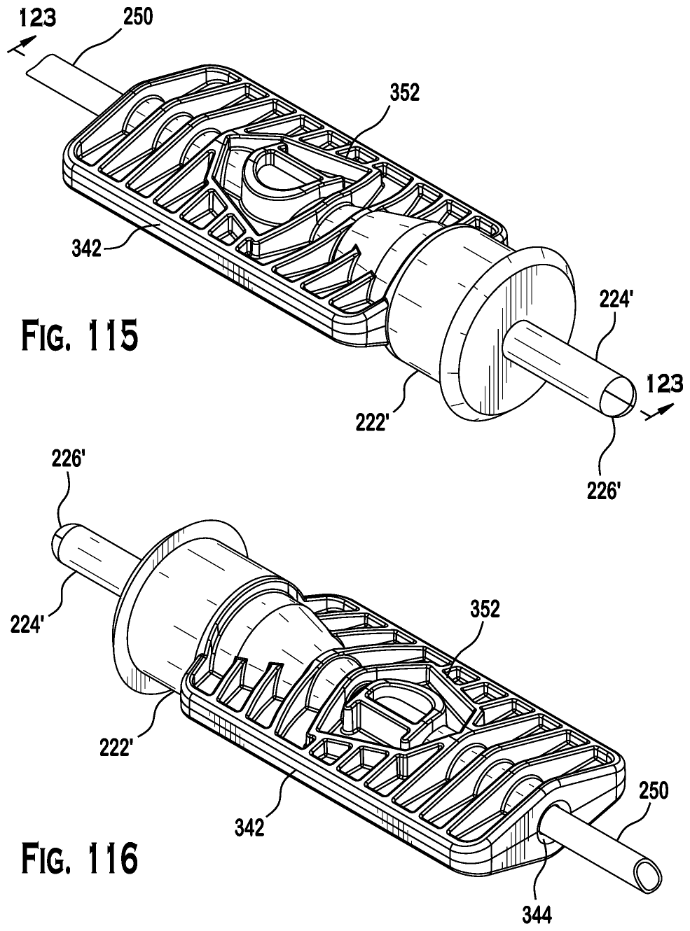

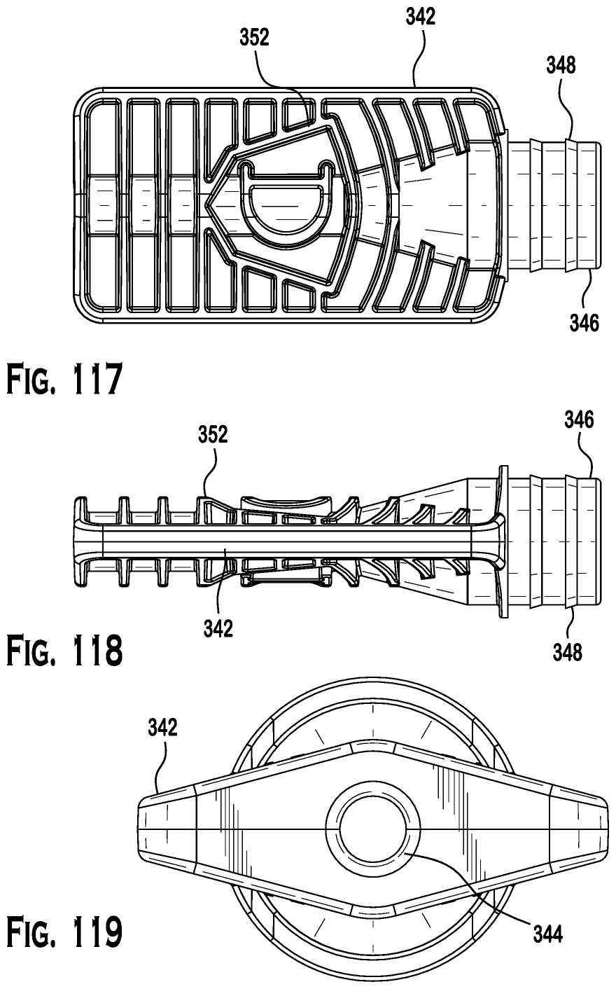

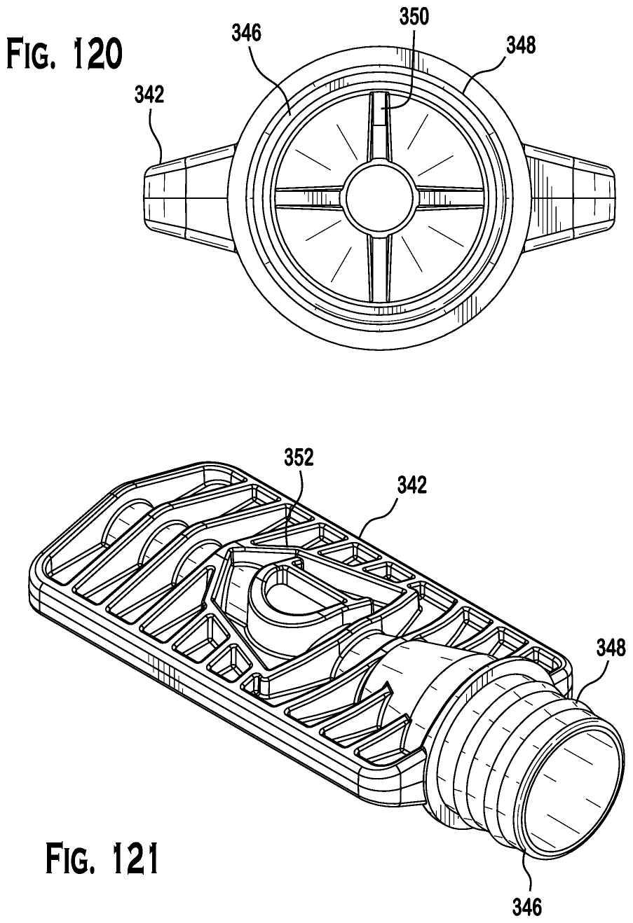

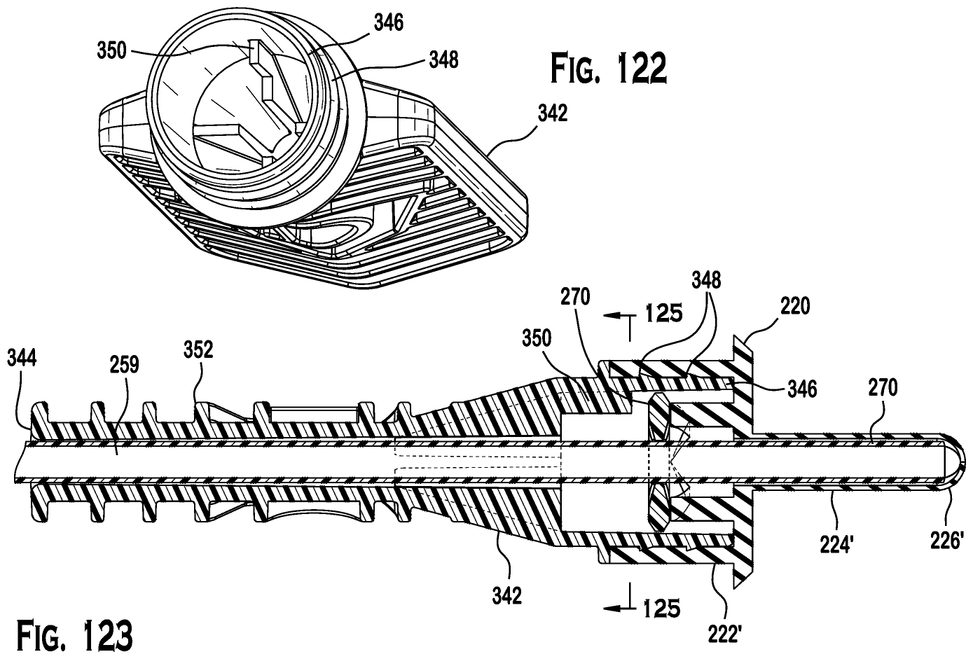

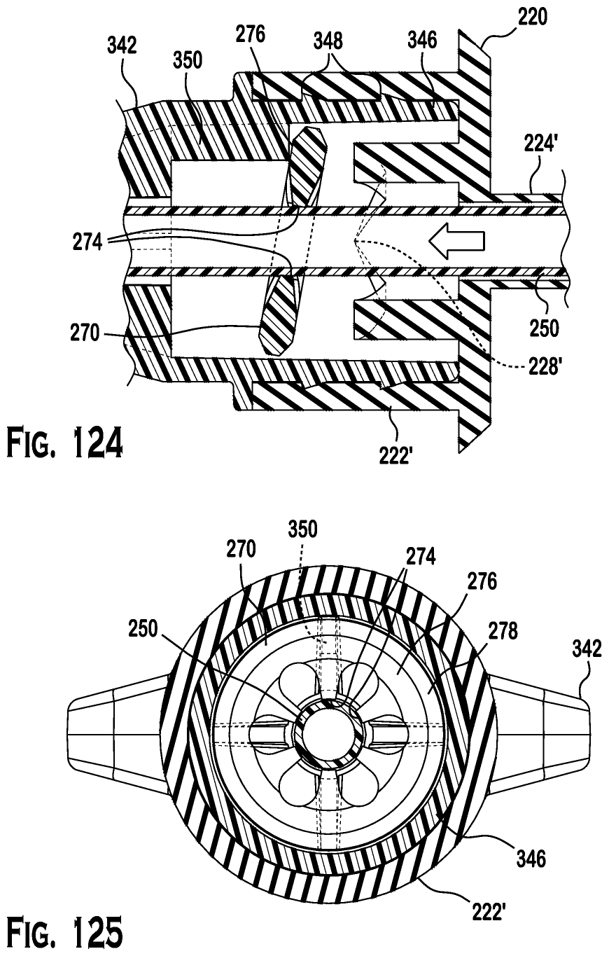

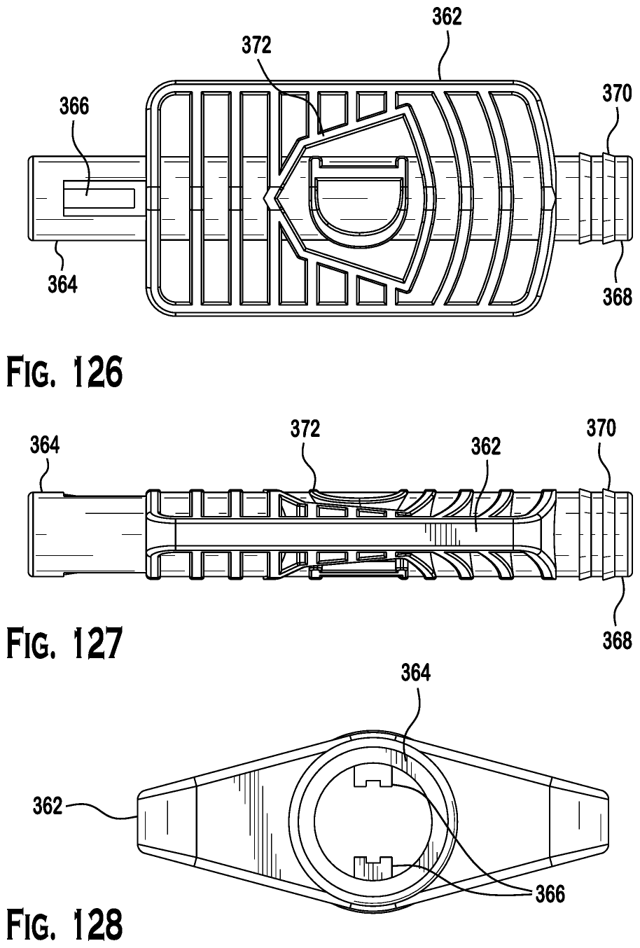

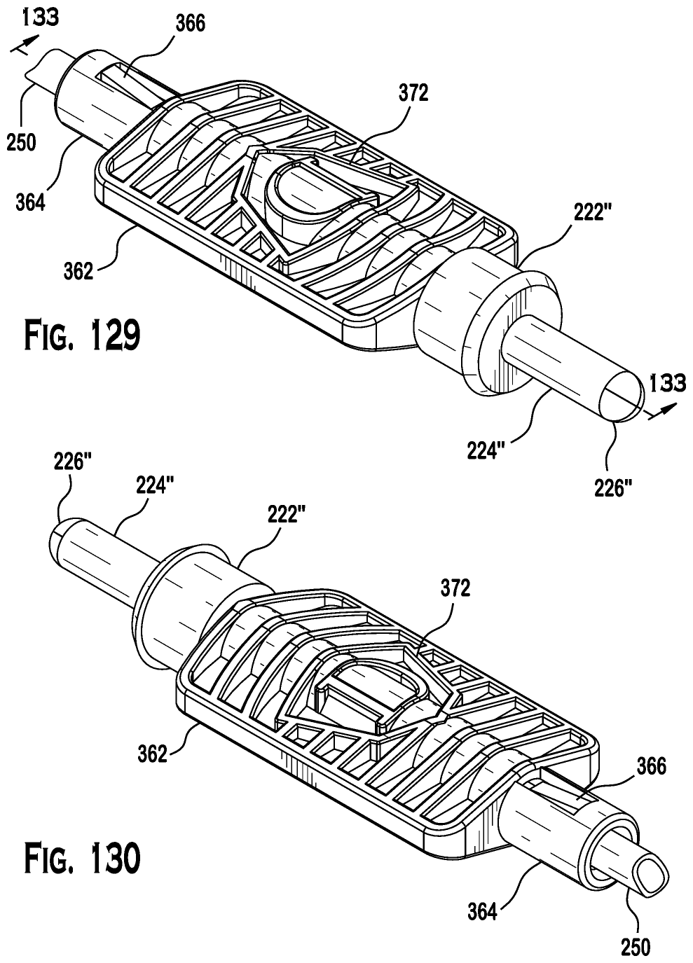

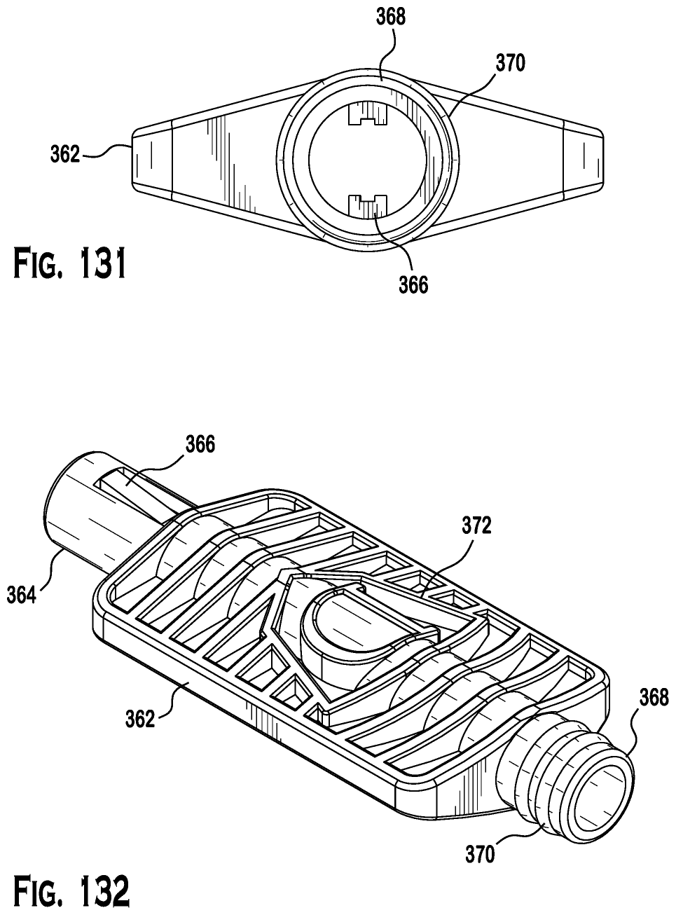

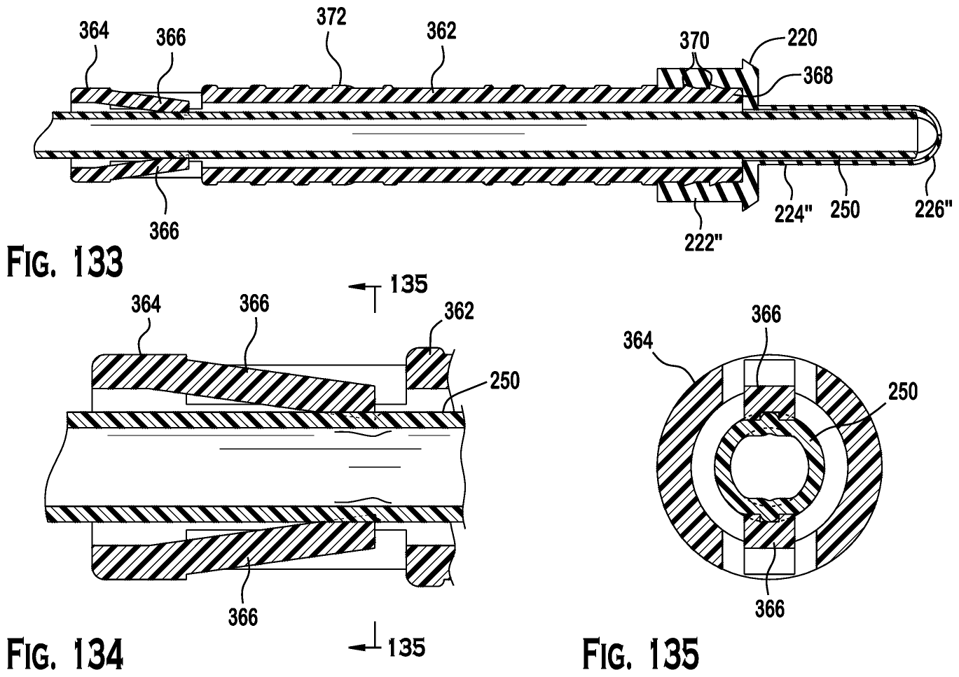

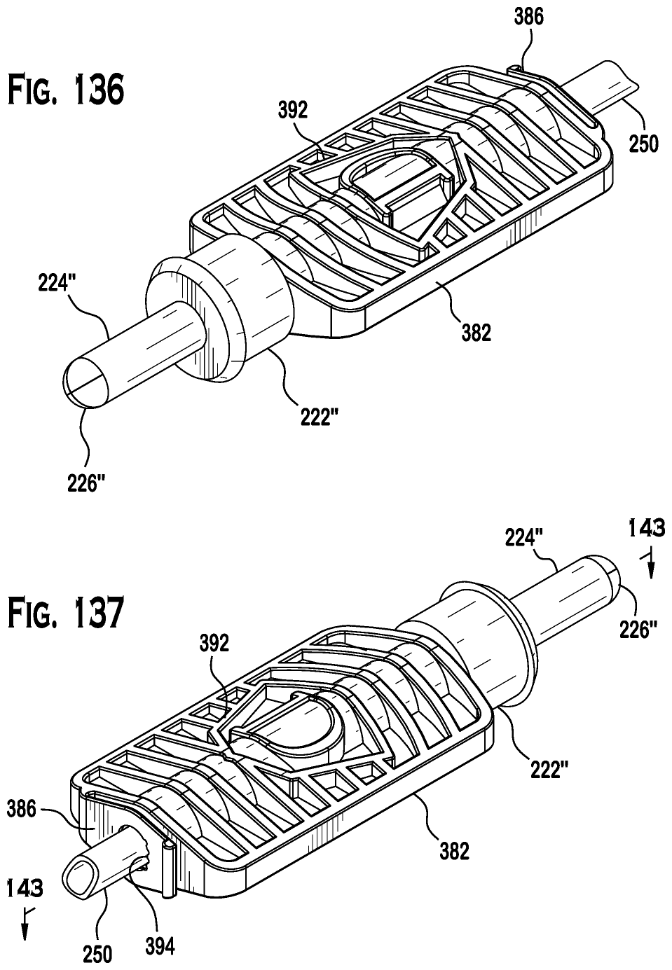

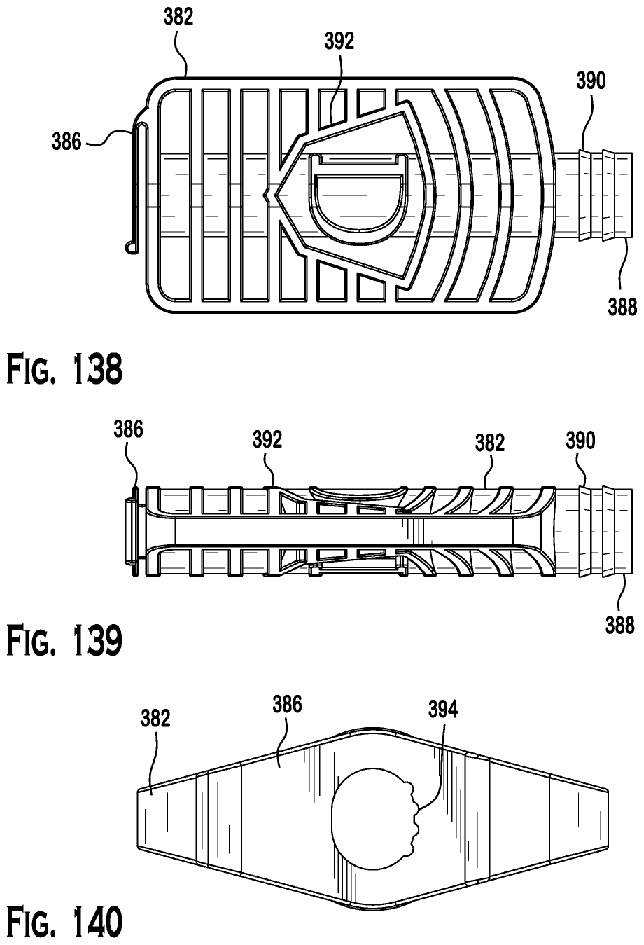

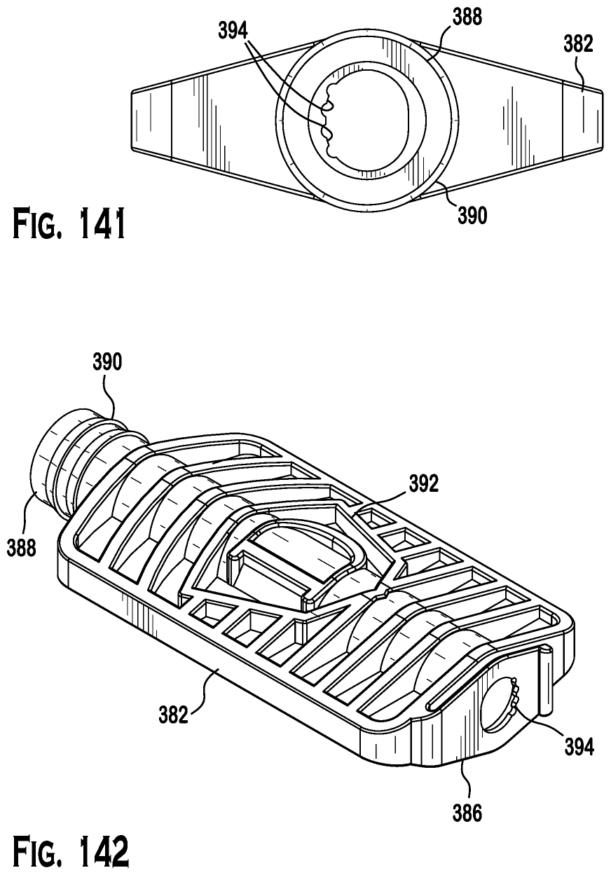

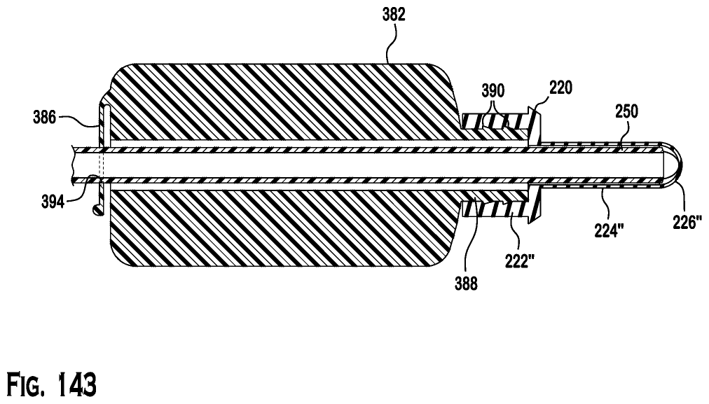

As such, in the first position, the catheter orifice 272 is sized such that the gripping surface 274 of the tube gripper 270 provides a first level of resistance to the movement of the catheter 250 relative to the housing receiving passageway 324, 344. While in the second position, the gripping surface 274 of the tube gripper 270 provides a second level of resistance to the movement of the catheter 250 relative to the housing receiving passageway 324,344. In this aspect, the second level of resistance is greater than the first level of resistance. In other words, the first level of resistance, in one aspect, is enough to lightly grip the catheter 250 such that the tube gripper 270 is moved, along with the catheter 250, from first position to second position as shown in FIGS. 100-103. One of ordinary skill in the art would understand, the biasing surface 228′ of the introducer tip 220′ increases the friction between the gripping surface 274 and the catheter 250.

When using the grip housing 360 with the catheter assembly 200. The protruding housing receiving passageway 364 includes a set of clasps 366. The clasps 366 prevent the catheter 250 from being disconnected from the housing 362 if there is an external force in either the first direction or the second direction. The bulbous ring 258 of the catheter 250 makes contact with the clasps 366 of the housing receiving passageway 364 which prevents the catheter 250 from being disconnected.

When using the grip housing 380 with the catheter assembly 200. The housing 382 includes a catheter securing flap 386. When the catheter 250 is passed through the catheter securing flap 386 with an external force in the first direction, the catheter 250 is given little or no resistance to pass through. When the catheter 250 is passed through the catheter securing flap 386 with an external force in the second direction, the catheter 250 is shown a greater level of resistance because of the grooved catheter grip 394 of the catheter securing flap 386. When a greater level of resistance on the catheter 250 is applied, it increases the friction. Further, when using an external force in the second direction, the grooved catheter grip 394 and the bulbous ring 258 of catheter 250 prevent the catheter 250 from being disconnected from the securing flap 386.

In the exemplary embodiment, once the urethra has been fully anesthetized, the catheter assembly 200, which would be connected to a sealed 500 ml rectangular bag (not shown) with a luer connector and a cap to receive urine

The first entry section 120 is in parallel communication with the catheter 9 and includes a device receiving passageway and a plug 122 to seal the device receiving passageway. The aspect of this device provides two (2) services, first, once the entire urethra has been anesthetized, removal of the plug 122 will allow the bladder to be fully emptied in preparation of the procedure to be conducted, and second, then becomes the proximal access to the protective sheath assembly 10 of this device which allows the introduction and passage of any suitable device 155 of FIG. 60, for example, an endoscope, and/or a biopsy needle/punch, surgical fiber and/or culture swab to safely.

The foregoing illustrates some of the possibilities for practicing the invention. Many other embodiments are possible within the scope and spirit of the invention. Therefore, more or less of the aforementioned components can be used to conform to that particular purpose. It is, therefore, intended that the foregoing description be regarded as illustrative rather than limiting, and that the scope of the invention is given by the appended claims together with their full range of equivalents.

What is claimed is:

1. A catheter assembly comprising:
a catheter having at least one connection fitting at a proximal end thereof and at least one opening positioned a distal end positioned opposite the proximal end;
a grip housing having a housing receiving passageway through which the catheter travels longitudinally there through, and a raised webbed grip positioned proximate an outer central region thereof;
a tube gripper movably positioned at one end of the grip housing and having a catheter orifice for receiving the catheter; and
an anesthetizing medicament device connected to the at least one connection fitting and provided with an anesthetizing medicament, the anesthetizing medicament being advanced by the anesthetizing medicament device through the catheter passage and exiting the at least one opening of the catheter.

2. The catheter assembly of claim 1, wherein the tube gripper includes a top face, a bottom face, and a gripping surface positioned between the top face and bottom face along the catheter orifice.

3. The catheter assembly of claim 2, wherein the tube gripper further includes a chamfered edge on the outer edge.

4. The catheter assembly of claim 3, wherein the tube gripper in a first position includes a portion of the top face making contact with the leveling surface such that a catheter orifice of the tube gripper is positioned substantially co-axial to the catheter passage.

5. The catheter assembly of claim 4, wherein the grip housing further includes an introducer tip connector positioned at a distal end thereof

6. The catheter assembly of claim 5, wherein the introducer tip connector further includes an elevated set of rings on the outer region and a leveling surface on the inner region.

7. The catheter assembly of claim 6, wherein the raised webbed grip further includes a raised D-shape icon in the central region on opposite faces of the grip housing.

8. The catheter assembly of claim 7, wherein the catheter assembly further includes an introducer tip positioned over the introducer tip connector.

9. The catheter assembly of claim 8, wherein the introducer tip includes an elongated cylindrical member, a protruding end piece and a tip connector catheter receiving passageway.

10. The catheter assembly of claim 9, wherein the introducer tip further includes a biasing surface with a set of protruding teeth located on the inner region of the elongated cylindrical member.

11. A catheter assembly comprising:
a catheter having a proximal end with at least one connection fitting and a distal end having at least one opening;
a grip housing having a protruding housing receiving passageway through which the catheter travels longitudinally there through, and a raised webbed grip proximate an outer central region thereof, the protruding housing receiving passageway having mirroring clasps located on opposite sides of the central region thereof; and
an anesthetizing medicament device connected to the at least one connection fitting and provided with an anesthetizing medicament, the anesthetizing medicament being advanced by the anesthetizing medicament device through the catheter passage and exiting the at least one opening of the catheter.

12. The catheter assembly of claim 11, wherein the grip housing further includes an introducer tip connector positioned at a distal end thereof.

13. The catheter assembly of claim 12, wherein the introducer tip connector includes an elevated set of rings on the outer region.

14. The catheter assembly of claim 13, wherein the raised webbed grip further includes a raised D-shape icon in the central region on opposite faces of the grip housing.

15. The catheter assembly of claim 14, wherein the catheter assembly further includes an introducer tip positioned over the introducer tip connector.

16. The catheter assembly of claim 15, wherein the introducer tip includes an elongated cylindrical member, a protruding end piece and a tip connector catheter receiving passageway.

17. A catheter assembly comprising:
a catheter having a proximal end with at least one connection fitting and a distal end having at least one opening;
a grip housing having:
a housing receiving passageway through which the catheter travels longitudinally there through;
a raised webbed grip proximate an outer central region thereof; and a catheter securing flap at a proximal end thereof; and
an anesthetizing medicament device connected to the at least one connection fitting and provided with an anesthetizing medicament, the anesthetizing medicament being advanced by the anesthetizing medicament device through the catheter passage and exiting the at least one opening of the catheter.

18. The catheter assembly of claim 17, wherein the catheter securing flap includes a grooved catheter grip located on the inner region.

19. The catheter assembly of claim 18, wherein the grip housing further includes an introducer tip connector at a distal end thereof

20. The catheter assembly of claim 19, wherein the introducer tip connector includes an elevated set of rings on the outer region.

21. The catheter assembly of claim 20, wherein the raised webbed grip further includes a D-shape icon in the central region on opposite faces of the grip housing.

22. The catheter assembly of claim 21, wherein the catheter assembly further includes an introducer tip positioned over the introducer tip connector.

23. The catheter assembly of claim 22, wherein the introducer tip includes an elongated cylindrical member, a protruding end piece and a tip connector catheter receiving passageway.

24. The catheter assembly of claim 23, wherein a sealed 500 mL rectangular bag is connected to a luer lock connector of the catheter assembly.
